SEQUENCE LISTING (87 sequences)

SEQ ID NO: 1 - protein, 159 aa, Escherichia coli
MISLIAALAVDHVIGMENAMPWNLPADLAWFKRNTLNKPVIMGRHTWESIGRPLPGRKNIILSSQPSTDDRVTWVKSVDEAIAACGDVPEIMVIGGGRVYEQFLPKAQKLYLTHIDAEVEGDTHFPDYEPDDWESVFSEFHDADAQNSHSYCFEILERR

SEQ ID NO: 2 - protein, 158 aa, Escherichia coli
ISLIAALAVDHVIGMETVMPWNLPADLAWFKRNTLNKPVIMGRHTWESIGRPLPGRKNIILSSQPSTDDRVTWVKSVDEAIAACGDVPEIMVIGGGRVYEQFLPKAQKLYLTHIDAEVEGDTHFPDYEPDDWESVFSEFHDADAQNSHSYCFEILERR

SEQ ID NO: 3 - DNA, 477 bp, Escherichia coli
  source 1..477: mol_type = genomic DNA
atcagtctgattgcggcgttagcggtagatcacgttatcggcatggaaaccgtcatgccgtggaacctgcctgccgatctcgcctggtttaaacgcaacaccttaaataaacccgtgattatgggccgccatacctgggaatcaatcggtcgtccgttgccaggacgcaaaaatattatcctcagcagtcaaccgagtacggacgatcgcgtaacgtgggtgaagtcggtggatgaagccatcgcggcgtgtggtgacgtaccagaaatcatggttattggcggcggtcgcgtttatgaacagttcttgccaaaagcgcaaaaactgtatctgacgcatatcgacgcagaagtggaaggcgacacccatttcccggattacgagccggatgactgggaatcggtattcagcgaattccacgatgctgatgcgcagaactctcacagctattgctttgagattctggagcggcgataa

SEQ ID NO: 4 - protein, 107 aa, unidentified
GVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLE

SEQ ID NO: 5 - protein, 875 aa, Homo sapiens
MERAGPSFGQQRQQQQPQQQKQQQRDQDSVEAWLDDHWDFTFSYFVRKATREMVNAWFAERVHTIPVCKEGIRGHTESCSCPLQQSPRADNSAPGTPTRKISASEFDRPLRPIVVKDSEGTVSFLSDSEKKEQMPLTPPRFDHDEGDQCSRLLELVKDISSHLDVTALCHKIFLHIHGLISADRYSLFLVCEDSSNDKFLISRLFDVAEGSTLEEVSNNCIRLEWNKGIVGHVAALGEPLNIKDAYEDPRFNAEVDQITGYKTQSILCMPIKNHREEVVGVAQAINKKSGNGGTFTEKDEKDFAAYLAFCGIVLHNAQLYETSLLENKRNQVLLDLASLIFEEQQSLEVILKKIAATIISFMQVQKCTIFIVDEDCSDSFSSVFHMECEELEKSSDTLTREHDANKINYMYAQYVKNTMEPLNIPDVSKDKRFPWTTENTGNVNQQCIRSLLCTPIKNGKKNKVIGVCQLVNKMEENTGKVKPFNRNDEQFLEAFVIFCGLGIQNTQMYEAVERAMAKQMVTLEVLSYHASAAEEETRELQSLAAAVVPSAQTLKITDFSFSDFELSDLETALCTIRMFTDLNLVQNFQMKHEVLCRWILSVKKNYRKNVAYHNWRHAFNTAQCMFAALKAGKIQNKLTDLEILALLIAALSHDLDHRGVNNSYIQRSEHPLAQLYCHSIMEHHHFDQCLMILNSPGNQILSGLSIEEYKTTLKIIKQAILATDLALYIKRRGEFFELIRKNQFNLEDPHQKELFLAMLMTACDLSAITKPWPIQQRIAELVATEFFDQGDRERKELNIEPTDLMNREKKNKIPSMQVGFIDAICLQLYEALTHVSEDCFPLLDGCRKNRQKWQALAEQQEKMLINGESGQAKRN

SEQ ID NO: 6 - protein, 833 aa, Homo sapiens
MLPFGDKTREMVNAWFAERVHTIPVCKEGIRGHTESCSCPLQQSPRADNSAPGTPTRKISASEFDRPLRPIVVKDSEGTVSFLSDSEKKEQMPLTPPRFDHDEGDQCSRLLELVKDISSHLDVTALCHKIFLHIHGLISADRYSLFLVCEDSSNDKFLISRLFDVAEGSTLEEVSNNCIRLEWNKGIVGHVAALGEPLNIKDAYEDPRFNAEVDQITGYKTQSILCMPIKNHREEVVGVAQAINKKSGNGGTFTEKDEKDFAAYLAFCGIVLHNAQLYETSLLENKRNQVLLDLASLIFEEQQSLEVILKKIAATIISFMQVQKCTIFIVDEDCSDSFSSVFHMECEELEKSSDTLTREHDANKINYMYAQYVKNTMEPLNIPDVSKDKRFPWTTENTGNVNQQCIRSLLCTPIKNGKKNKVIGVCQLVNKMEENTGKVKPFNRNDEQFLEAFVIFCGLGIQNTQMYEAVERAMAKQMVTLEVLSYHASAAEEETRELQSLAAAVVPSAQTLKITDFSFSDFELSDLETALCTIRMFTDLNLVQNFQMKHEVLCRWILSVKKNYRKNVAYHNWRHAFNTAQCMFAALKAGKIQNKLTDLEILALLIAALSHDLDHRGVNNSYIQRSEHPLAQLYCHSIMEHHHFDQCLMILNSPGNQILSGLSIEEYKTTLKIIKQAILATDLALYIKRRGEFFELIRKNQFNLEDPHQKELFLAMLMTACDLSAITKPWPIQQRIAELVATEFFDQGDRERKELNIEPTDLMNREKKNKIPSMQVGFIDAICLQLYEALTHVSEDCFPLLDGCRKNRQKWQALAEQQEKMLINGESGQAKRN

SEQ ID NO: 7 - protein, 823 aa, Homo sapiens
MVNAWFAERVHTIPVCKEGIRGHTESCSCPLQQSPRADNSAPGTPTRKISASEFDRPLRPIVVKDSEGTVSFLSDSEKKEQMPLTPPRFDHDEGDQCSRLLELVKDISSHLDVTALCHKIFLHIHGLISADRYSLFLVCEDSSNDKFLISRLFDVAEGSTLEEVSNNCIRLEWNKGIVGHVAALGEPLNIKDAYEDPRFNAEVDQITGYKTQSILCMPIKNHREEVVGVAQAINKKSGNGGTFTEKDEKDFAAYLAFCGIVLHNAQLYETSLLENKRNQVLLDLASLIFEEQQSLEVILKKIAATIISFMQVQKCTIFIVDEDCSDSFSSVFHMECEELEKSSDTLTREHDANKINYMYAQYVKNTMEPLNIPDVSKDKRFPWTTENTGNVNQQCIRSLLCTPIKNGKKNKVIGVCQLVNKMEENTGKVKPFNRNDEQFLEAFVIFCGLGIQNTQMYEAVERAMAKQMVTLEVLSYHASAAEEETRELQSLAAAVVPSAQTLKITDFSFSDFELSDLETALCTIRMFTDLNLVQNFQMKHEVLCRWILSVKKNYRKNVAYHNWRHAFNTAQCMFAALKAGKIQNKLTDLEILALLIAALSHDLDHRGVNNSYIQRSEHPLAQLYCHSIMEHHHFDQCLMILNSPGNQILSGLSIEEYKTTLKIIKQAILATDLALYIKRRGEFFELIRKNQFNLEDPHQKELFLAMLMTACDLSAITKPWPIQQRIAELVATEFFDQGDRERKELNIEPTDLMNREKKNKIPSMQVGFIDAICLQLYEALTHVSEDCFPLLDGCRKNRQKWQALAEQQEKMLINGESGQAKRN

SEQ ID NO: 8 - protein, 326 aa, Homo sapiens
EETRELQSLAAAVVPSAQTLKITDFSFSDFELSDLETALCTIRMFTDLNLVQNFQMKHEVLCRWILSVKKNYRKNVAYHNWRHAFNTAQCMFAALKAGKIQNKLTDLEILALLIAALSHDLDHRGVNNSYIQRSEHPLAQLYCHSIMEHHHFDQCLMILNSPGNQILSGLSIEEYKTTLKIIKQAILATDLALYIKRRGEFFELIRKNQFNLEDPHQKELFLAMLMTACDLSAITKPWPIQQRIAELVATEFFDQGDRERKELNIEPTDLMNREKKNKIPSMQVGFIDAICLQLYEALTHVSEDCFPLLDGCRKNRQKWQALAEQQ

SEQ ID NO: 9 - protein, 260 aa, Homo sapiens
MSHHWGYGKHNGPEHWHKDFPIAKGERQSPVDIDTHTAKYDPSLKPLSVSYDQATSLRILNNGHAFNVEFDDSQDKAVLKGGPLDGTYRLIQFHFHWGSLDGQGSEHTVDKKKYAAELHLVHWNTKYGDFGKAVQQPDGLAVLGIFLKVGSAKPGLQKVVDVLDSIKTKGKSADFTNFDPRGLLPESLDYWTYPGSLTTPPLLECVTWIVLKEPISVSSEQVLKFRKLNFNGEGEPEELMVDNWRPAQPLKNRQIKASFK

SEQ ID NO: 10 - protein, 505 aa, Homo sapiens
MGETLGDSPIDPESDSFTDTLSANISQEMTMVDTEMPFWPTNFGISSVDLSVMEDHSHSFDIKPFTTVDFSSISTPHYEDIPFTRTDPVVADYKYDLKLQEYQSAIKVEPASPPYYSEKTQLYNKPHEEPSNSLMAIECRVCGDKASGFHYGVHACEGCKGFFRRTIRLKLIYDRCDLNCRIHKKSRNKCQYCRFQKCLAVGMSHNAIRFGRMPQAEKEKLLAEISSDIDQLNPESADLRALAKHLYDSYIKSFPLTKAKARAILTGKTTDKSPFVIYDMNSLMMGEDKIKFKHITPLQEQSKEVAIRIFQGCQFRSVEAVQEITEYAKSIPGFVNLDLNDQVTLLKYGVHEIIYTMLASLMNKDGVLISEGQGFMTREFLKSLRKPFGDFMEPKFEFAVKFNALELDDSDLAIFIAVIILSGDRPGLLNVKPIEDIQDNLLQALELQLKLNHPESSQLFAKLLQKMTDLRQIVTEHVQLLQVIKKTETDMSLHPLLQEIYKDLY

SEQ ID NO: 11 - protein, 189 aa, Homo sapiens
SVEAVQEITEYAKSIPGFVNLDLNDQVTLLKYGVHEIIYTMLASLMNKDGVLISEGQGFMTREFLKSLRKPFGDFMEPKFEFAVKFNALELDDSDLAIFIAVIILSGDRPGLLNVKPIEDIQDNLLQALELQLKLNHPESSQLFAKLLQKMTDLRQIVTEHVQLLQVIKKTETDMSLHPLLQEIYKDLY

SEQ ID NO: 12 - protein, 231 aa, Homo sapiens
MAGKKVLIVYAHQEPKSFNGSLKNVAVDELSRQGCTVTVSDLYAMNLEPRATDKDITGTLSNPEVFNYGVETHEAYKQRSLASDITDEQKKVREADLVIFQFPLYWFSVPAILKGWMDRVLCQGFAFDIPGFYDSGLLQGKLALLSVTTGGTAEMYTKTGVNGDSRYFLWPLQHGTLHFCGFKVLAPQISFAPEIASEEERKGMVAAWSQRLQTIWKEEPIPCTAHWHFGQ

SEQ ID NO: 13 - protein, 245 aa, Homo sapiens
SLALSLTADQMVSALLDAEPPILYSEYDPTRPFSEASMMGLLTNLADRELVHMINWAKRVPGFVDLTLHDQVHLLECAWLEILMIGLVWRSMEHPGKLLFAPNLLLDRNQGKCVEGMVEIFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKGMEHLYSMKCKNVVPLYDLLLEMLDAHRL

SEQ ID NO: 14 - protein, 139 aa, Anguilla japonica
MVEKFVGTWKIADSHNFGEYLKAIGAPKELSDGGDATTPTLYISQKDGDKMTVKIENGPPTFLDTQVKFKLGEEFDEFPSDRRKGVKSVVNLVGEKLVYVQKWDGKETTYVREIKDGKLVVTLTMGDVVAVRSYRRATE

SEQ ID NO: 15 - protein, 8 aa, unidentified
  VARIANT 4: note = Any amino acid
DVEXNPGP

SEQ ID NO: 16 - protein, 19 aa, Teschovirus A
ATNFSLLKQAGDVEENPGP

SEQ ID NO: 17 - protein, 22 aa, synthetic construct
GSGATNFSLLKQAGDVEENPGP

SEQ ID NO: 18 - protein, 20 aa, Equine rhinitis A virus
QCTNYALLKLAGDVESNPGP

SEQ ID NO: 19 - protein, 23 aa, synthetic construct
GSGQCTNYALLKLAGDVESNPGP

SEQ ID NO: 20 - protein, 22 aa, Foot-and-mouth disease virus
VKQTLNFDLLKLAGDVESNPGP

SEQ ID NO: 21 - protein, 25 aa, synthetic construct
GSGVKQTLNFDLLKLAGDVESNPGP

SEQ ID NO: 22 - protein, 18 aa, Thosea asigna virus
EGRGSLLTCGDVEENPGP

SEQ ID NO: 23 - protein, 21 aa, synthetic construct
GSGEGRGSLLTCGDVEENPGP

SEQ ID NO: 24 - protein, 7 aa, synthetic construct
DLLPRGT

SEQ ID NO: 25 - DNA, 21 bp, synthetic construct
  source 1..21: mol_type = other DNA
gatctgctgccacgcgggacg

SEQ ID NO: 26 - protein, 7 aa, synthetic construct
DLLPRGS

SEQ ID NO: 27 - protein, 10 aa, synthetic construct
STQELLRFPK

SEQ ID NO: 28 - protein, 9 aa, synthetic construct
KLSAREQRD

SEQ ID NO: 29 - protein, 11 aa, synthetic construct
CSVEDLLKFEK

SEQ ID NO: 30 - protein, 9 aa, synthetic construct
KGSKEEQRD

SEQ ID NO: 31 - protein, 7 aa, synthetic construct
ELLPKGS

SEQ ID NO: 32 - protein, 18 aa, synthetic construct
MEGISNFKTPSKLSEKKK

SEQ ID NO: 33 - protein, 12 aa, synthetic construct
MSRRKQAKPQHI

SEQ ID NO: 34 - protein, 12 aa, synthetic construct
HYPWFKARLYPL

SEQ ID NO: 35 - protein, 12 aa, synthetic construct
ETFSDLWKLLPE

SEQ ID NO: 36 - protein, 12 aa, synthetic construct
TSFAEYWNLLSP

SEQ ID NO: 37 - protein, 12 aa, synthetic construct
ETFEHWWSQLLS

SEQ ID NO: 38 - protein, 12 aa, synthetic construct
ETFEHWWAQLTS

SEQ ID NO: 39 - protein, 12 aa, synthetic construct
LTFEHSWAQLTS

SEQ ID NO: 40 - protein, 12 aa, synthetic construct
LTFEHWWAQLTS

SEQ ID NO: 41 - protein, 6 aa, synthetic construct
TPDYFL

SEQ ID NO: 42 - protein, 24 aa, synthetic construct
VKKKKIKREIKIFRGRSRFRGRSR

SEQ ID NO: 43 - protein, 18 aa, synthetic construct
RQKRLIRQKRLIRQKRLI

SEQ ID NO: 44 - protein, 27 aa, synthetic construct
VKKKKIKREIKIPRRPGPTRKHYQPYA

SEQ ID NO: 45 - protein, 20 aa, synthetic construct
PLTAVFWLIYVLAKALVTVC

SEQ ID NO: 46 - protein, 48 aa, synthetic construct
SVDDHFAKSLGDTWLQIGGSGNPKTANVPQTVPMRLRKLPDSFFKPPE

SEQ ID NO: 47 - protein, 48 aa, synthetic construct
SVDDHFAKSLGDTWLQIGGSGNPKTANVPQTVPARLRKLPDSAFKPPE

SEQ ID NO: 48 - protein, 48 aa, synthetic construct
SVDDAAAKSLGDTWLQIGGSGNPKTANVPQTVPARLRKLPDSAFKPPE

SEQ ID NO: 49 - protein, 52 aa, synthetic construct
DPVVEEHFRRSLGKNYKEPEPAPNSVSITGSVDDHFAKALGDTWLQIKAAKD

SEQ ID NO: 50 - protein, 17 aa, synthetic construct
DPVVEEHFRRSLGKNYK

SEQ ID NO: 51 - protein, 18 aa, synthetic construct
DPVVEEHFRRSLGKNYKE

SEQ ID NO: 52 - protein, 20 aa, synthetic construct
DPVVEEHFRRSLGKNYKEPE

SEQ ID NO: 53 - protein, 24 aa, synthetic construct
SVSITGSVDDHFAKALGDTWLQIK

SEQ ID NO: 54 - protein, 25 aa, synthetic construct
SVSITGSVDDHFAKALGDTWLQIKA

SEQ ID NO: 55 - protein, 28 aa, synthetic construct
SVSITGSVDDHFAKALGDTWLQIKAAKD

SEQ ID NO: 56 - protein, 17 aa, synthetic construct
SVDDHFAKSLGDTWLQI

SEQ ID NO: 57 - protein, 15 aa, synthetic construct
LALRLACIGDEMDVS

SEQ ID NO: 58 - protein, 15 aa, synthetic construct
EKAELLQGGDLLRQR

SEQ ID NO: 59 - protein, 15 aa, synthetic construct
VESILKKNSDWIWDW

SEQ ID NO: 60 - protein, 15 aa, synthetic construct
EVEALKKSADWVSDW

SEQ ID NO: 61 - protein, 15 aa, synthetic construct
TAARLKALGDELHQR

SEQ ID NO: 62 - protein, 15 aa, synthetic construct
IAQELRRIGDEFNAY

SEQ ID NO: 63 - protein, 15 aa, synthetic construct
YGRELRRMSDEFVDS

SEQ ID NO: 64 - protein, 15 aa, synthetic construct
IARHLAQVGDSMDRS

SEQ ID NO: 65 - protein, 15 aa, synthetic construct
IARKLQCIADQFHRL

SEQ ID NO: 66 - protein, 15 aa, synthetic construct
CATQLRRFGDKLNFR

SEQ ID NO: 67 - protein, 15 aa, synthetic construct
IGAQLRRMADDLNAQ

SEQ ID NO: 68 - protein, 15 aa, synthetic construct
LSRRLKVTGDLFDIM

SEQ ID NO: 69 - protein, 15 aa, synthetic construct
IVELLKYSGDQLERK

SEQ ID NO: 70 - protein, 15 aa, synthetic construct
ALETLRRVGDGVQRN

SEQ ID NO: 71 - protein, 15 aa, synthetic construct
IGSKLAAMCDDFDAQ

SEQ ID NO: 72 - protein, 15 aa, synthetic construct
IGRKLTVMCDEFDSE

SEQ ID NO: 73 - protein, 15 aa, synthetic construct
NIRRLRALADGVQKV

SEQ ID NO: 74 - protein, 15 aa, synthetic construct
NIDKLRALADDIDKT

SEQ ID NO: 75 - protein, 15 aa, synthetic construct
MVTLLPIEGQEIHFF

SEQ ID NO: 76 - protein, 15 aa, synthetic construct
PTVPLPSETDGYVAP

SEQ ID NO: 77 - protein, 15 aa, synthetic construct
PQRYLVIQGDDRMKL

SEQ ID NO: 78 - protein, 15 aa, synthetic construct
TVGELSRALGHENGS

SEQ ID NO: 79 - protein, 15 aa, synthetic construct
VGQLLQDMGDDVYQQ

SEQ ID NO: 80 - protein, 15 aa, synthetic construct
LHEVLNGLLDRPDWE

SEQ ID NO: 81 - protein, 15 aa, synthetic construct
AVHSLSRIGDELYLE

SEQ ID NO: 82 - protein, 15 aa, synthetic construct
NPKFLKNAGRDCSRR

SEQ ID NO: 83 - protein, 16 aa, synthetic construct
ERLRRRIELCRRHHST

SEQ ID NO: 84 - protein, 9 aa, synthetic construct
KCILCRLLQ

SEQ ID NO: 85 - protein, 10 aa, synthetic construct
MFPHRVTAZK

SEQ ID NO: 86 - protein, 12 aa, synthetic construct
LTFEHYWAQLTS

SEQ ID NO: 87 - protein, 12 aa, synthetic construct
TSFAEYWNLLSP

(19) United States
(12) Patent Application Publication
(10) Pub. No.: US 2026/0242414 A1
(43) Pub. Date: Aug. 20, 2026

(54) PEPTIDE EXPRESSION CONSTRUCTS AND USES THEREOF

(71) Applicant: KYOTO PREFECTURAL PUBLIC UNIVERSITY CORPORATION, Kyoto (JP)

(72) Inventor: Satoshi GOJO, Kyoto (JP)

(73) Assignee: KYOTO PREFECTURAL PUBLIC UNIVERSITY CORPORATION, Kyoto (JP)

(21) Appl. No.: 19/164,887

(22) PCT Filed: Mar. 14, 2024
(86) PCT No.: PCT/IB2024/052507

§ 371 Date: Sep. 12, 2025

Related U.S. Application Data
(60) Provisional application No. 63/490,324, filed on Mar. 15, 2023.

(51) Int. Cl. C07K 7/06; A61K 38/08; A61K 38/44; A61K 48/00; C07K 7/08; C07K 14/00; C12N 9/06

(52) U.S. Cl. CPC C07K 7/06; A61K 38/08; A61K 38/44; A61K 48/005; C07K 7/08; C07K 14/00; C12N 9/003; C12Y 105/01003

(57) ABSTRACT

The disclosure provides gene transfer constructs comprising a destabilizing domain (DD) sequence, a translational separator sequence, and a sequence encoding one or more copies of one or more peptides of interest; nucleic acids encoding gene transfer constructs; uses of nucleic acids encoding gene transfer constructs to prevent mitochondrial hyperfission and fragmentation; uses of nucleic acids encoding gene transfer constructs to induce apoptosis in cells, such as cancer cells; and therapeutic applications of nucleic acids encoding gene transfer constructs, for example, in the treatment of mitochondrial diseases and disorders associated with mitochondrial dysfunction, as well as various types of cancer.

### 1. CROSS-REFERENCE TO RELATED APPLICATIONS

This application claims the priority benefit of U.S. provisional application No. 63/490,324, filed Mar. 15, 2023, the contents of which are incorporated herein in their entireties by reference thereto.

### 2. SEQUENCE LISTING

The instant application contains a Sequence Listing which has been submitted electronically in XML format and is hereby incorporated by reference in its entirety. Said XML Sequence Listing, created on Feb. 29, 2024, is named RMG-002WO_SL.XML and is 82,183 bytes in size.

### 3. BACKGROUND

Many physiological processes are regulated through protein complexes, which typically involve protein-protein interactions (PPIs). Thus, numerous diseases can be traced to aberrant PPIs, including metabolic diseases, cancer, infectious diseases, and neurodegenerative diseases (Lu et al., 2020, Sig Transduct and Target Ther 5:213). Although many aberrant PPIs involve the weakening or loss of an essential interaction, numerous others involve PPIs at an inappropriate time, location, or between unlikely partners.

Targeting aberrant PPIs is a promising intervention strategy for various diseases, yet it remains a challenge due to large, flat, and relatively hydrophobic PP interface areas. One strategy is to design PPI modulators that act on PPI interface hot spots, which are the amino acid residues that contribute significantly to binding. For instance, small peptides generated with the amino acid sequence of a PPI hot spot can act as competitive inhibitors. In fact, several small peptides have been developed and shown to be effective as PPI inhibitors in cultured cells (Lu et al., 2020. Sig Transduct and Target Ther 5:213; Chang et al., 2013. PNAS 110:E3445-E3454).

Although peptides mimicking PPI interface hot spots have high target specificity and affinity, challenges stand in the way of their use as PPI modulators, such as limited biological availability upon oral administration, poor solubility, and low membrane permeance.

Therefore, novel approaches for providing short peptide modulators of PPI are needed.

### 4. SUMMARY

Intracellular expression of short peptides, particularly peptides shorter than 50 amino acids in length, is challenging, as ribosome binding to mRNAs decreases as coding sequence length decreases. This disclosure is based, in part, on the discovery that intracellular expression of short peptides can be promoted using nucleic acid molecules encoding a nucleotide sequence encoding a short peptide linked to a nucleotide sequence encoding a destabilizing domain (DD) such as E. coli DHFR via a translational separator such as the self-cleaving peptide P2A. Without being bound by theory, it is believed that the sequence encoding the DD can act as a ribosome binding fragment, leading to increased expression of the peptide of interest compared to a nucleic acid not including the DD sequence. In the absence of the DD's stabilizing molecule, the DD is degraded after translation.

Accordingly, in one aspect, the disclosure provides a mRNA molecule encoding (a) a destabilizing domain (DD), (b) a translational separator, and (c) a peptide of interest.

Exemplary features of mRNA molecules of the disclosure are described in Sections 6.2 and 6.3 and specific embodiments 1 to 101, infra.

In another aspect, the disclosure provides DNA molecules encoding an mRNA of the disclosure. For example, the DNA can be an expression vector such as a plasmid, or a viral genome such as an AAV genome.

Exemplary features of DNA molecules of the disclosure are described in Section 6.3, including Section 6.3.1.3, and specific embodiments 102 to 110, infra.

In another aspect, the disclosure provides particles comprising the nucleic acids. For instance, nucleic acid-comprising particles can be viral particles (e.g., retroviral particles or AAV particles) or lipid particles (e.g., lipid nanoparticles).

Exemplary features of particles of the disclosure are described in Section 6.3, including Sections 6.3.1.3 and 6.3.3, and specific embodiment 111, infra.

In further aspects, the disclosure provides host cells comprising a nucleic acid of the disclosure. Host cells can be prokaryotic or eukaryotic, and can be used to, for example, to propagate a nucleic acid of the disclosure, or to propagate and package a particle of the disclosure, or to express a peptide of interest.

Exemplary features of host cells of the disclosure are described in Section 6.3.2 and specific embodiment 113, infra.

In another aspect, the disclosure provides pharmaceutical compositions comprising a nucleic acid of the disclosure, or a cell of the disclosure, and a pharmaceutically acceptable excipient.

Exemplary features of pharmaceutical compositions of the disclosure are further described in Section 6.5 and specific embodiment 112, infra.

In another aspect, the disclosure provides methods of introducing nucleic acids into host cells. The disclosure also provides methods of expressing peptides of interest in host cells. In further aspects, the disclosure provides methods of (a) preventing mitochondrial hyperfission, and/or (b) inducing apoptosis in cancer cells. In yet another aspect, the disclosure provides methods of treating a subject with nucleic acids or pharmaceutical compositions of the disclosure.

Further exemplary features of methods of the disclosure are described in Sections 6.3.3 and 6.4, and specific embodiments 114 to 133, infra.

### 5. BRIEF DESCRIPTION OF THE FIGURES

FIG. 1 is a cartoon illustrating regulation of the stability of a dihydrofolate reductase (DHFR)-fused protein. In the absence of a stabilizing drug, such as trimethoprim (TMP), the destabilizing domain (DD) of DHFR targets the fusion protein to the ubiquitin proteasome system (UPS) for degradation. When TMP is added, it binds to the destabilizing domain of DHFR and inhibits the UPS-mediated degradation of the fusion protein.

FIGS. 2A-2F show exemplary peptide expression units of the disclosure. FIG. 2A is a vector diagram illustrating the general organization of individual components of exemplary peptide expression units of the disclosure, comprising the coding sequences of an optional marker, DHFR, a translational separator (e.g., P2A), and a peptide of interest, incorporated between a promoter and a polyA sequence. FIGS. 2B and 2C show the diagrams of two peptide expression units that were evaluated. FIGS. 2D and 2E depict control constructs. FIG. 2F shows the pCAGGS expression plasmid, which comprises the peptide expression unit depicted in FIG. 2B.

FIG. 3 shows the pLenti-GFPP1-10-puro plasmid used to generate GFPP1-10-expressing cells.

FIG. 4 is a cartoon illustration of the split green fluorescent protein (GFP) system. A functional GFP has 11 β strands. The split GFP system relies on the interaction of two non-fluorescing polypeptides, GFP β strand 1-10 (GFPP1-10) and GFP β strand 11 (GFPβ11), to form a fluorescent GFP molecule.

FIG. 5 shows fluorescence microscopy images in GFP+ cells and A549 cells transfected with the GFPP1-10-comprising plasmid depicted in FIG. 3. A549 cells transfected with GFPP1-10 plasmids had robust levels of GFPP1-10 mRNA but lacked both GFP signal and GFP mRNA, indicating these cells do not express full-length GFP.

FIGS. 6A-6I show the results of transfection of GFPP-1-10-expressing A549 cells with plasmids depicted in FIGS. 2B, 2D, and 2E relative to non-transfected cells. FIG. 6A shows fluorescence microscopy images in each group of cells. FIG. 6B shows the results of FACS analysis. FIGS. 6C, 6D, and 6E show the percentage of cells in each group expressing mCherry, GFP, or both mCherry and GFP, respectively. FIG. 6F shows the GFP intensity in cells in each group. FIGS. 6G-6I display levels of mRNA relative to GAPDH expression in cells in each group.

FIGS. 7A-7F show the effect of TMP on peptide expression in cells transfected with a plasmid comprising an mCherry-DHFR-P2A-GFPβ11 peptide expression unit. FIG. 7A shows GFP and mCherry fluorescent microscopy images in the absence and presence of TMP in cells transfected with a plasmid comprising an mCherry-DHFR-P2A-GFPβ11. FIG. 7B shows the results of FACS analysis of cells transfected with a plasmid comprising mCherry-DHFR-P2A-GFPβ11. FIGS. 7C, 7D, and 7E show the percentage of cells expressing mCherry, GFP, or both mCherry and GFP, respectively, in the presence or absence of TMP. FIG. 7F shows the results of FACS analysis of cells transfected with plasmids comprising mCherry, GFP, GFP-G7-mCherry, and GFP-P2A-mCherry.

FIGS. 8A-8N show that incorporation of P110 protects cells against LPS-induced mitochondrial fragmentation. FIG. 8A shows GFP and mCherry fluorescent microscopy images in untreated control H9c2 cells and in H9c2 cells lipofected with the mRNA of a GFPβ11-comprising construct or a P110-comprising construct. FIGS. 8B and 8C show the results of FACs analysis and mean fluorescence intensity of GFP in the same groups of cells. FIG. 8D shows the fluorescence microscopy images of cells in each treatment group stained with mitotracker green. FIGS. 8E-81 display the results of Mitochondrial Network Analysis (MiNA). FIGS. 8J-8N show the results of reactive oxygen species (ROS) and mitochondrial membrane potential assessments.

FIGS. 9A-9O show that incorporation of P110 protects cells against doxorubicin-induced mitochondrial fragmentation and apoptosis. FIG. 9A shows the low magnification (left panels) and high magnification (right panels) fluorescence microscopy images of cells in each treatment group stained with mitotracker green. The boxes on the left panels indicate areas that are magnified on the right. FIGS. 9B-9F display the results of MiNA for each treatment group. FIG. 9G show the results of FACS analysis, wherein the upper left, upper right, and lower right quadrants in each assessment indicate necrotic cells, cells in late apoptosis, and cells in early apoptosis, respectively. FIG. 9H is a graph depicting the percentage of cells in late apoptosis in each treatment group. FIGS. 9I-9O show the results of mitochondrial respiration assessments via flux analysis.

FIGS. 10A-10K show that incorporation of P110 protects against cell death associated with mitochondrial dysfunction. FIGS. 10A-10B show the results of FACS analysis of apoptosis using DAPI and annexin V. FIGS. 10C-10H show the results of western blot analyses of apoptosis-associated proteins. FIGS. 10I-10K show the results of immunoprecipitation via Fis1 antibody, analyzed by western blot.

### 6. DETAILED DESCRIPTION

### 6.1. Definitions

Unless defined otherwise, all technical and scientific terms used herein have the same meaning as commonly understood to one of ordinary skill in the art to which this disclosure belongs. The following definitions are provided for the full understanding of terms used in this specification.

As used herein, the following terms are intended to have the following meanings:

A, An, The: As used herein, the term “a”, “an”, “the” and similar terms used in the context of the present disclosure (especially in the context of the claims) are to be construed to cover both the singular and plural unless otherwise indicated herein or clearly contradicted by the context. As such, the terms “a” (or “an”), “one or more”, and “at least one” can be used interchangeably herein.

And/or: The term “and/or” means that each one or both or all the components or features of a list are possible variants, especially two or more thereof in an alternative or cumulative way.

Apoptosis: As used herein, “apoptosis” refers to a form of cell death in which a programmed sequence of events leads to the death of a cell. Hallmarks of apoptosis include morphological changes, cell shrinkage, caspase activation, nuclear and cytoplasmic condensation, and alterations in plasma membrane topology. Biochemically, apoptotic cells are characterized by increased intracellular calcium concentration, fragmentation of chromosomal DNA, and expression of novel cell surface components. In some embodiments, a cell undergoing apoptosis may undergo mitochondrial outer membrane permeabilization (MOMP).

Cancer: As used herein, “cancer” is a condition involving abnormal and/or unregulated cell growth. The term cancer encompasses benign and malignant cancers. Exemplary cancers include pancreatic cancer (e.g., pancreatic ductal adenocarcinoma), lung cancer, small cell lung cancer or non-small cell lung cancer, e.g., lung adenocarcinoma, colorectal cancer, melanoma (e.g., having a BRAF mutation), leukemia, e.g., acute myeloid leukemia or acute lymphocytic leukemia, lymphoma, e.g., non-Hodgkin lymphoma, diffuse large B-Cell lymphoma (DLBCL), myeloma, e.g., multiple myeloma, leiomyosarcoma, breast cancer, liver cancer, osteosarcoma, and head and neck cancer.

Destabilization Domain (DD): The term “destabilization domain” or “destabilizing domain” refers to a polypeptide domain that is, in the absence of a stabilizing molecule, unstable and readily ubiquitinated and degraded by proteasomes. Degradation of a DD-containing peptide or fusion polypeptide can be prevented by a suitable stabilizing agent. Exemplary destabilization domains include dihydrofolate reductase (DHFR) destabilization domains (which can be stabilized by the exemplary stabilizing agent trimethoprim), FK506-binding protein (FKBP) destabilization domains (which can be stabilized by the exemplary stabilizing agents Shield-1 (Shld1), rapamycin and FK506), PDE5 destabilization domains (which can be stabilized by the exemplary stabilizing agents sildenafil, vardenafil, tadalafil, avanafil, lodenafil, mirodenafil, udenafil, benzamidenafil, dasantafil, and beminafil), CA2 destabilization domains (which can be stabilized by the exemplary stabilizing agents Celecoxib (Celebrex), Valdecoxib, Rofecoxib (Vioxx), Acetazolamide, Methazolamide, Dorzolamide, Brinzolamide, Diclofenamide, Ethoxzolamide, Zonisamide, Dansylamide, and Dichlorphenamide), PPAR gamma destabilizing domains (which can be stabilized by the exemplary stailizing agents Pioglitazone and Posiglitazone), NQO2 destabilizing domains (which can be stabilized by the exemplary stabilizing agents Imatinib and Melatonin), ERLBD destabilizing domains (which can be stabilized by the exemplary stabilizing agents CMP8, 4-hydroxytamoxifen (Afimoxifene), tamoxifen, fulvestrant, and raloxifene), and UnaG destabilizing domains (which can be stabilized by the exemplary stabilizing agent bilirubin). Exemplary DHFR destabilization domains are described in Iwamoto et al., 2010, Chem Biol. 17(9):981-8, Liu et al., 2014 Int. J. Parasitol. 44(10):729-735, and U.S. Pat. No. 9,487,787; exemplary FKBP destabilization domains are described in Banaszynski et al., 2006, Cell 126(5):995-1004 and U.S. Pat. No. 9,487,787; and exemplary PDE5 destabilization domains are described in WO 2018/237323; exemplary CA2 destabilization domains are described in U.S. Pat. No. 9,487,787; exemplary PPAR gamma destabilizing domains and NQO2 destabilizing domains are described in WO 2018/160993 A1 and US 2022/0213449 A1, exemplary ERLBD destabilizing domains are described in Miyazaki et al., 2012, J Am Chem Soc. 134(9): 3942-3945 and in US 2014/0255361 A1; and exemplary UnaG destabilization domains are described in Navarro et al., 2016, ACS Chem Biol. 11(8):2101-4, the contents of each of which are incorporated herein by reference in their entireties.

Effective Amount: The term “effective amount” or “therapeutically effective amount” means the amount or quantity of an agent or composition that is sufficient to elicit the required or desired response, or in other words, the amount that is sufficient to elicit an appreciable biological response when administered to a subject (e.g., amelioration of one or more signs or symptoms of a disease or disorder or improvement in a biomarker associated with a disease or disorder). Said amount preferably relates to an amount that is therapeutically or in a broader sense also prophylactically effective against the progression of a disease or disorder as disclosed herein. It is understood that an “effective amount” or a “therapeutically effective amount” can vary from subject to subject, due to variation in metabolism of an agent, age, weight, general condition of the subject, the condition being treated, the severity of the condition being treated, and the judgment of the prescribing physician.

mRNA: As used herein, the term “mRNA” refers to a messenger ribonucleic acid. Unless required otherwise by context, the term “mRNA” encompasses modified and unmodified mRNAs. A modified mRNA can include, for example, one or more modified and/or non-naturally occurring components such as one or more non-naturally occurring nucleobases, nucleosides, nucleotides, or internucleoside linkages. An mRNA can include a cap structure, a chain terminating nucleoside, a stem loop, a polyA sequence, and/or a polyadenylation signal. Traditionally, the basic components of an mRNA molecule include at least a coding region, a 5′-untranslated region (5′-UTR), a 3UTR, a 5′ cap and a polyadenylation (polyA) sequence.

mmRNA: As used herein, “modified mRNA” or “mmRNA” refers to an mRNA molecule having at least one modified sugar group, nucleobase, and/or internucleoside linkage. In one embodiment, the mRNA molecules of the disclosure are modified by the introduction of non-natural nucleosides and/or nucleotides, e.g., as it relates to the natural ribonucleotides A, U, G, and C. Noncanonical nucleotides, such as cap structures, are not considered “modified” although they differ from the chemical structure of the A, C, G, U ribonucleotides.

Or: Unless indicated otherwise, an “or” conjunction is intended to be used in its correct sense as a Boolean logical operator, encompassing both the selection of features in the alternative (A or B, where the selection of A is mutually exclusive from B) and the selection of features in conjunction (A or B, where both A and B are selected). In some places in the text, the term “and/or” is used for the same purpose, which shall not be construed to imply that “or” is used with reference to mutually exclusive alternatives.

Peptide: The term “peptide” refers to a molecule comprising two or more amino acids linked by the carboxyl group of one amino acid to the alpha amino group of another. Peptides of the disclosure are typically two to 150 amino acids in length, for example three to 100, four to 50, 5 to 20, or 6 to 10 amino acids in length.

Percentage Identity: Percentage identity between two amino acid sequences or nucleotide sequences is calculated by multiplying the number of matches between a pair of aligned sequences by 100, and dividing by the length of the aligned region. Identity scoring only counts perfect matches, and it does not consider substitutions or deletions as matches. For calculation of the percent sequence identity, two sequences are aligned using the EMBOSS Needle Pairwise Sequence Alignment software tool based on the Needleman and Wunsch algorithm (available at www.ebi.ac.uk/Tools/psa/emboss_needle) with the following parameters: Matrix: BLOSUM62 (for protein sequences) or DNAfull (for DNA sequences); Gap Open: 10; Gap Extend: 0.5; End Gap Penalty: false; End Gap Open: 10; and End Gap Extend: 0.5

Subject: As used herein, the term “subject” refers to any organism to which a composition in accordance with the disclosure may be administered, e.g., for diagnostic, prophylactic, and/or therapeutic purposes. In some embodiments, the subject is a human.

Transfection: As used herein, the term “transfection” refers to methods to introduce a species (e.g., a polynucleotide, such as a mRNA) into a cell.

Translational Separator: As used herein, the term “translational separator” refers to a sequence that enables the generation of distinct peptide products from a single mRNA molecule that comprises multiple coding sequences. A translational separator can be a self-cleaving peptide, which allows for expression of discrete peptide or protein products from a single mRNA molecule through ribosomal skipping. Without being bound by theory, self-cleaving peptides are thought to function by inducing the ribosome to skip the synthesis of a peptide bond at their C-termini. This peptide bond skipping leads to separation between, for example, the end of the self-cleaving peptide sequence and the next peptide downstream, without preventing downstream translation.

Treat, Treating, Treatment: The terms “treat,” “treating,” “treatment,” and grammatical variations thereof as used herein, include reducing or ameliorating a disease or disorder, and/or signs or symptoms associated therewith, or slowing or halting the progression thereof. It will be appreciated that, although not precluded, treating a disease or disorder does not require that the disease, disorder or symptoms associated therewith be completely eliminated. Treatments according to the disclosure may be applied prophylactically (e.g., to a subject at risk of developing a disease or disorder), palliatively or remedially. Prophylactic treatments can be administered to a subject prior to onset of a sign or symptom, during early onset of a sign or symptom (e.g., upon initial signs and symptoms), or after an established development of a sign or symptom. Prophylactic administration can occur for several days to years prior to the manifestation of a symptom.

### 6.2. Gene Transfer Constructs

The present disclosure provides gene transfer constructs, e.g., mRNAs, comprising a destabilizing domain (DD) sequence, a translational separator sequence, and a sequence encoding one or more copies of one or more peptides of interest. Exemplary features of destabilizing domains, translational separators, and peptides of interest are described in Sections 6.2.1, 6.2.2, and 6.2.3, respectively.

The DD sequence and the peptide of interest sequence can be positioned on either side of the translational separator sequence. In some embodiments, the gene transfer construct is an mRNA molecule comprising, in the 5′ to 3′ order, a peptide of interest sequence, a translational separator sequence, and DD sequence. In preferred embodiments, the gene transfer construct is an mRNA molecule comprising, in the 5′ to 3′ order, a DD sequence, a translational separator sequence, and a peptide of interest sequence.

### 6.2.1. Destabilizing Domain

The gene transfer constructs of the disclosure include a sequence encoding a destabilizing domain (DD), which (without being bound by theory) serves as a ribosome binding fragment and enables the translation of the gene transfer construct mRNA. Exemplary DDs include DDs derived from dihydrofolate reductase (DHFR), FK506-binding protein (FKBP), phosphodiesterase 5 (PDE5), carbonic anhydrase 2 (CA2), peroxisome proliferator-activated receptor gamma (PPAR gamma), NRH:quinone oxidoreductase 2 (NQO2), human estrogen receptor ligand binding domain (ERLBD), and UnaG.

Exemplary DHFR DDs are described in U.S. Pat. No. 9,487,787, the contents of which are incorporated herein in their entirety. An amino acid sequence of wild-type E. coli DHFR is as follows:

(SEQ ID NO: 1)
MISLIAALAVDHVIGMENAMPWNLPADLAWFKRNTLNKPVIMGRHTWESIGRPLPGR
KNIILSSQPSTDDRVTWVKSVDEAIAACGDVPEIMVIGGGRVYEQFLPKAQKLYLTH
IDAEVEGDTHFPDYEPDDWESVESEFHDADAQNSHSYCFEILERR

A DHFR DD can comprise a wild-type DHFR sequence or can comprise one or more amino acid substitutions and/or truncations at the N and/or C terminal end. For example, a DHFR DD sequence can be at least 80%, at least 85%, at least 90%, at least 95%, at least 96%, at least 97%, at least 98%, at least 99% or 100% identical to SEQ ID NO:1. Exemplary amino acid substitutions and combinations that can be included in a DHFR DD include Y100I, G121V, N18T/A19V, F103L, H12Y/Y100I, H12L/Y100I, R98H/F103S, M42T/H114R, I61F/T68S, and W74R/T113S/E120D/Q146L (Nakahara et al., 2022, ACS Chem Biol 17:2877-2889). Combinations of the foregoing substitutions can also be used. In some embodiments, the DHFR comprises an amino acid sequence which is identical to SEQ ID NO:1 except for a Y100I, G121V, N18T/A19V, F103L, H12Y/Y100I, H12L/Y100I, R98H/F103S, M42T/H114R, I61F/T68S, or W74R/T113S/E120D/Q146L substitution(s), or a combination thereof. In some embodiments, a DHFR DD lacks an N-terminal methionine. For example, in some embodiments, the DHFR comprises an amino acid sequence which is identical to SEQ ID NO:1 except for a Y100I, G121V, N18T/A19V, F103L, H12Y/Y100I, H12L/Y100I, R98H/F103S, M42T/H114R, I61F/T68S, or W74R/T113S/E120D/Q146L substitution(s), or a combination thereof, and lack of the N-terminal methionine.

In some embodiments, a DHFR DD has an amino acid sequence that is at least 80%, at least 85%, at least 90%, at least 95%, or 100% identical to the following sequence:

(SEQ ID NO: 2)
ISLIAALAVDHVIGMETVMPWNLPADLAWFKRNTLNKPVIMGRHTWESIGRPLPGRK
NIILSSQPSTDDRVTWVKSVDEAIAACGDVPEIMVIGGGRVYEQFLPKAQKLYLTHI
DAEVEGDTHFPDYEPDDWESVFSEFHDADAONSHSYCFEILERR.

An exemplary nucleotide sequence encoding SEQ ID NO:2 is the following:

(SEQ ID NO: 3)
Atcagtctgattgcggcgttagcggtagatcacgttatcggcatggaaaccgtcatg
ccgtggaacctgcctgccgatctcgcctggtttaaacgcaacaccttaaataaaccc
gtgattatgggccgccatacctgggaatcaatcggtcgtccgttgccaggacgcaaa
aatattatcctcagcagtcaaccgagtacggacgatcgcgtaacgtgggtgaagtcg
gtggatgaagccatcgcggcgtgtggtgacgtaccagaaatcatggttattggcggc
ggtcgcgtttatgaacagttcttgccaaaagcgcaaaaactgtatctgacgcatatc
gacgcagaagtggaaggcgacacccatttcccggattacgagccggatgactgggaa
tcggtattcagcgaattccacgatgctgatgcgcagaactctcacagctattgcttt
gagattctggagcggcgataa.

An exemplary stabilizing agent for a DHFR DD is trimethoprim (TMP). Another exemplary stabilizing agent for a DHFR DD is methotrexate (MTX).

Exemplary FKBP DDs are described in U.S. Pat. No. 9,487,787, the contents of which are incorporated herein in their entirety. An amino acid sequence of an exemplary FKBP DD (having a F36V substitution compared to the wild-type sequence) is as follows:

(SEQ ID NO: 4)
GVQVETISPGDGRTFPKRGQTCVVHYTGMLEDGKKVDSSRDRNKPFKFMLGKQEVIR
GWEEGVAQMSVGORAKLTISPDYAYGATGHPGIIPPHATLVEDVELLKLE

A FKBP DD can comprise a wild-type FKBP sequence or can comprise one or more amino acid substitutions. For example, a FKBP DD sequence can be at least 80%, at least 85%, at least 90%, at least 95%, at least 96%, at least 97%, at least 98%, at least 99% or 100% identical to SEQ ID NO:4. Exemplary amino acid substitutions that can be included in a FKBP DD include F15S, V24A, H25R, E60G, L106P, D100G, M66T, R71G, D100N, E102G, and K1051. Combinations of the foregoing substitutions can also be used. In some embodiments, the DD comprises an amino acid sequence which is identical to SEQ ID NO:4 except for a F15S, V24A, H25R, E60G, L106P, D100G, M66T, R71G, D100N, E102G, or K1051 substitution, or a combination thereof.

Exemplary FKBP DD stabilizing agents include Shield-1 (Shld1), rapamycin, and FK506.

Exemplary PDE5 DDs are described in WO 2018/237323, the contents of which are incorporated herein in their entirety. PDE5 DDs may be derived from PDE5A, Isoform 1 (SEQ ID NO:5); PDE5A Isoform 2 (SEQ ID NO:6) and/or PDE5A Isoform 3 (SEQ ID NO:7). These isoforms differ at their N-terminal regions and have unique first exons followed by a common sequence of 823 amino acids.

All PDE5A isoforms contain a catalytic domain that is located near the C-terminus of the protein and is relatively selective for cGMP as a substrate at physiological levels. The substrate binding site is also the binding site for several known PDE5 inhibitors such as sildenafil, which have been utilized to treat cardiovascular diseases and erectile dysfunction. Towards the N-terminus, two homologous GAF domains are located. One of the GAF domains, GAF-A contains a high affinity binding site for cGMP. Occupancy of this domain by cGMP is known to cause activation of the catalytic domain. Moreover, the affinity of this site for cGMP is increased by cGMP-dependent protein kinase-mediated phosphorylation of serine 92. In another embodiment, a PDE5A DD can comprise the catalytic domain of PDE5A, spanning from amino acid position 535 to position 860 of UniProt ID: 076074 (SEQ ID NO:5), as represented in SEQ ID NO:8. In addition to the catalytic domain, PDE5A DDs may also comprise one or more GAF domains and/or the C terminal portion that extends beyond the catalytic domain. In one embodiment, the PDE5A derived DD comprises amino acids from position 535 to position 875 of SEQ ID NO:5. In another embodiment, the PDE5 DD comprises amino acids from position 466 to 875 or position 420 to 875 of SEQ ID NO:5. Exemplary PDE5 DD sequences are set forth in Table 1.

TABLE 1

| PDE5 DD | Amino Acid Sequence | SEQ ID NO: |
|---|---|---|
| PDE5A | MERAGPSFGQQRQQQQPQQQKQQQRDQDSVEAWLDDHWDFTFSYFVRKAT | 5 |
| Isoform 1 | REMVNAWFAERVHTIPVCKEGIRGHTESCSCPLQQSPRADNSAPGTPTRK | |
| | ISASEFDRPLRPIVVKDSEGTVSFLSDSEKKEQMPLTPPRFDHDEGDQCS | |
| | RLLELVKDISSHLDVTALCHKIFLHIHGLISADRYSLFLVCEDSSNDKFL | |
| | ISRLFDVAEGSTLEEVSNNCIRLEWNKGIVGHVAALGEPLNIKDAYEDPR | |
| | FNAEVDQITGYKTQSILCMPIKNHREEVVGVAQAINKKSGNGGTFTEKDE | |
| | KDFAAYLAFCGIVLHNAQLYETSLLENKRNQVLLDLASLIFEEQQSLEVI | |
| | LKKIAATIISFMQVQKCTIFIVDEDCSDSFSSVFHMECEELEKSSDTLTR | |
| | EHDANKINYMYAQYVKNTMEPLNIPDVSKDKRFPWTTENTGNVNQQCIRS | |
| | LLCTPIKNGKKNKVIGVCQLVNKMEENTGKVKPFNRNDEQFLEAFVIFCG | |
| | LGIQNTQMYEAVERAMAKQMVTLEVLSYHASAAEEETRELQSLAAAVVPS | |
| | AQTLKITDFSFSDFELSDLETALCTIRMFTDLNLVQNFQMKHEVLCRWIL | |
| | SVKKNYRKNVAYHNWRHAFNTAQCMFAALKAGKIQNKLTDLEILALLIAA | |
| | LSHDLDHRGVNNSYIQRSEHPLAQLYCHSIMEHHHFDQCLMILNSPGNQI | |
| | LSGLSIEEYKTTLKIIKQAILATDLALYIKRRGEFFELIRKNQFNLEDPH | |
| | QKELFLAMLMTACDLSAITKPWPIQQRIAELVATEFFDQGDRERKELNIE | |
| | PTDLMNREKKNKIPSMQVGFIDAICLQLYEALTHVSEDCFPLLDGCRKNR | |
| | QKWQALAEQQEKMLINGESGQAKRN | |
| PDE5A | MLPFGDKTREMVNAWFAERVHTIPVCKEGIRGHTESCSCPLQQSPRADNS | 6 |
| Isoform 2 | APGTPTRKISASEFDRPLRPIVVKDSEGTVSFLSDSEKKEQMPLTPPRFD | |
| | HDEGDQCSRLLELVKDISSHLDVTALCHKIFLHIHGLISADRYSLFLVCE | |
| | DSSNDKFLISRLFDVAEGSTLEEVSNNCIRLEWNKGIVGHVAALGEPLNI | |
| | KDAYEDPRFNAEVDQITGYKTQSILCMPIKNHREEVVGVAQAINKKSGNG | |
| | GTFTEKDEKDFAAYLAFCGIVLHNAQLYETSLLENKRNQVLLDLASLIFE | |
| | EQQSLEVILKKIAATIISFMQVQKCTIFIVDEDCSDSFSSVFHMECEELE | |
| | KSSDTLTREHDANKINYMYAQYVKNTMEPLNIPDVSKDKRFPWTTENTGN | |
| | VNQQCIRSLLCTPIKNGKKNKVIGVCQLVNKMEENTGKVKPFNRNDEQFL | |
| | EAFVIFCGLGIQNTQMYEAVERAMAKQMVTLEVLSYHASAAEEETRELQS | |
| | LAAAVVPSAQTLKITDESFSDFELSDLETALCTIRMFTDLNLVQNFQMKH | |
| | EVLCRWILSVKKNYRKNVAYHNWRHAFNTAQCMFAALKAGKIQNKLTDLE | |
| | ILALLIAALSHDLDHRGVNNSYIQRSEHPLAQLYCHSIMEHHHFDQCLMI | |
| | LNSPGNQILSGLSIEEYKTTLKIIKQAILATDLALYIKRRGEFFELIRKN | |
| | QFNLEDPHQKELFLAMLMTACDLSAITKPWPIQQRIAELVATEFFDQGDR | |
| | ERKELNIEPTDLMNREKKNKIPSMQVGFIDAICLQLYEALTHVSEDCFPL | |
| | LDGCRKNRQKWQALAEQQEKMLINGESGQAKRN | |
| PDE5A | MVNAWFAERVHTIPVCKEGIRGHTESCSCPLQQSPRADNSAPGTPTRKIS | 7 |
| Isoform 3 | ASEFDRPLRPIVVKDSEGTVSFLSDSEKKEQMPLTPPRFDHDEGDQCSRL | |
| | LELVKDISSHLDVTALCHKIFLHIHGLISADRYSLFLVCEDSSNDKFLIS | |
| | RLFDVAEGSTLEEVSNNCIRLEWNKGIVGHVAALGEPLNIKDAYEDPRFN | |
| | AEVDQITGYKTQSILCMPIKNHREEVVGVAQAINKKSGNGGTFTEKDEKD | |
| | FAAYLAFCGIVLHNAQLYETSLLENKRNQVLLDLASLIFEEQQSLEVILK | |
| | KIAATIISFMQVQKCTIFIVDEDCSDSFSSVFHMECEELEKSSDTLTREH | |
| | DANKINYMYAQYVKNTMEPLNIPDVSKDKRFPWTTENTGNVNQQCIRSLL | |
| | CTPIKNGKKNKVIGVCQLVNKMEENTGKVKPFNRNDEQFLEAFVIFCGLG | |
| | IQNTQMYEAVERAMAKQMVTLEVLSYHASAAEEETRELQSLAAAVVPSAQ | |
| | TLKITDFSFSDFELSDLETALCTIRMFTDLNLVQNFQMKHEVLCRWILSV | |
| | KKNYRKNVAYHNWRHAFNTAQCMFAALKAGKIQNKLTDLEILALLIAALS | |
| | HDLDHRGVNNSYIQRSEHPLAQLYCHSIMEHHHFDQCLMILNSPGNQILS | |
| | GLSIEEYKTTLKIIKQAILATDLALYIKRRGEFFELIRKNQFNLEDPHQK | |
| | ELFLAMLMTACDLSAITKPWPIQQRIAELVATEFFDQGDRERKELNIEPT | |
| | DLMNREKKNKIPSMQVGFIDAICLQLYEALTHVSEDCFPLLDGCRKNRQK | |
| | WQALAEQQEKMLINGESGQAKRN | |
| Catalytic | EETRELQSLAAAVVPSAQTLKITDESFSDFELSDLETALCTIRMFTDLNL | 8 |
| domain of | VQNFQMKHEVLCRWILSVKKNYRKNVAYHNWRHAFNTAQCMFAALKAGKI | |
| PDE5A | QNKLTDLEILALLIAALSHDLDHRGVNNSYIQRSEHPLAQLYCHSIMEHH | |
| (position | HFDQCLMILNSPGNQILSGLSIEEYKTTLKIIKQAILATDLALYIKRRGE | |
| 535 to | FFELIRKNQFNLEDPHQKELFLAMLMTACDLSAITKPWPIQQRIAELVAT | |
| position | EFFDQGDRERKELNIEPTDLMNREKKNKIPSMQVGFIDAICLQLYEALTH | |
| 860 of | VSEDCFPLLDGCRKNRQKWQALAEQQ | |
| UniProt ID: | | |
| O76074 | | |
| (SEQ ID | | |
| NO. 5)) | | |

Exemplary amino acid substitutions that can be included in PDE5 DDs include one or more amino acid substitutions selected from E535D, E536G, Q541R, K555R, F559L S560G, F561 L, F564L, F564S, V585A, N587S, K591E, I599V, K604E, K608E, N609H, K630R, K633E, N636S, I648V, N661S, S663P, L675P, Y676D, Y676N, C677R, H678R, D687A, T711A, T712S, D724N, L738H, N742S, F744L, L746S, F755L, A762S, D764V, D764N, D764G, S766F, K795E, L797F, I799T, L804P, T802P, S815C, M816A, M816T, I824T, C839S, F840S, and K852E. The PDE5 DDs can also contain additional substitutions such as Q589R. In some embodiments, a PDE5 DD sequence comprises a sequence selected from the group of amino acid sequences identified by SEQ ID NOs. 19-35 of WO 2018/237323 and SEQ ID NOs.66-69 of WO 2018/237323.

Exemplary stabilizing agents for PDE5 DDs include sildenafil, vardenafil, tadalafil, avanafil, lodenafil, mirodenafil, udenafil, benzamidenafil, dasantafil, and beminafil.

Exemplary CA2 DDs are described in WO 2020/185632, the contents of which are incorporated herein in their entirety. An amino acid sequence of wild-type human CA2, corresponding to the GenBank Access NO. P00918, is as follows:

(SEQ ID NO: 9)
MSHHWGYGKHNGPEHWHKDFPIAKGERQSPVDIDTHTAKYDPSLKPLSVSYDQATSL
RILNNGHAFNVEFDDSQDKAVLKGGPLDGTYRLIQFHFHWGSLDGQGSEHTVDKKKY
AAELHLVHWNTKYGDFGKAVQQPDGLAVLGIFLKVGSAKPGLQKVVDVLDSIKTKGK
SADFTNFDPRGLLPESLDYWTYPGSLTTPPLLECVTWIVLKEPISVSSEQVLKFRKL
NENGEGEPEELMVDNWRPAQPLKNRQIKASFK

A CA2-derived DD can comprise a wild-type CA2 sequence or can comprise one or more amino acid substitutions and/or truncations at the N and/or C terminal end. For example, a CA2-derived DD sequence can be at least 80%, at least 85%, at least 90%, at least 95%, at least 96%, at least 97%, at least 98%, at least 99% or 100% identical to SEQ ID NO:9. Exemplary amino acid substitutions and combinations that can be included in a CA2 DD include A115L, A116Q, A116V, A133L, A133T, A141P, A152D, A152L, A152R, A173C, A173G, A173L, A173T, A23P, A247L, A247S, A257L, A257S, A38P, A38V, A54Q, A54V, A54X, A65L, A65N, A65V, A77I, A77P, A77Q, C205M, C205R, C205V, C205W, C205Y, D101G, D101M, D110I, D129I, D138G, D138M, D138N, D161*, D161M, D161V, D164G, D164I, D174*, D174T, D179E, D179I, D179R, D189G, D189I, D19T, D19V, D242G, D242T, D32T, D34T, D41T, D52I, D52L, D71F, D71G, D71K, D71M, D71S, D71Y, D72I, D72S, D72T, D72X, D75T, D75V, D85M, E106D, E106G, E106S, E117*, E117N, E14N, E186*, E186N, E204A, E204D, E204G, E204N, E213*, E213G, E213N, E220K, E220R, E220S, E233D, E233G, E233R, E235*, E235G, E235N, E237K, E237R, E238*, E238N, E238R, E26S, E69D, E69K, E69S, F130L, F146V, F175I, F175L, F175S, F178L, F178S, F20L, F20S, F225I, F225L, F225S, F225Y, F230I, F230L, F230S, F259L, F259S, F66S, F70I, F70L, F95Y, G102D, G104R, G104V, G128R, G12D, G12E, G131E, G131R, G131W, G139D, G144D, G144V, G150A, G150S, G150W, G155A, G155C, G155D, G155S, G170A, G170D, G182A, G182W, G195A, G195R, G232R, G232W, G234L, G234V, G25E, G63D, G63V, G81E, G81V, G82D, G86A, G86D, G98V, H107I, H107Q, H119T, H119Y, H122T, H122Y, H15L, H15T, H15Y, H17D, H17I, H36I, H36Q, H64M, H94T, H96T, I145F, I145M, I166H, I166L, I209D, I209L, I215H, I215S, I22L, I255N, I255S, I33S, I59F, I59N, I59S, I91F, K111E, K111N, K112R, K113I, K113N, K126N, K132E, K132R, K148E, K148R, K153*, K153N, K158E, K158N, K167*, K169N, K169R, K171Q, K171R, K18R, K212N, K212Q, K212R, K212W, K224E, K224N, K227*, K227N, K24R, K251E, K251R, K256Q, K260F, K260L, K260Q, K39S, K45N, K45S, K80M, K80R, L118F, L120W, L140V, L140W, L143*, L147*, L147F, L156F, L156H, L156P, L156Q, L163A, L163W, L183P, L183S, L184F, L184P, L188P, L188W, L197*, L197M, L197P, L197R, L197T, L202F, L202H, L202I, L202P, L202R, L202S, L203P, L203S, L203W, L211*, L211A, L211S, L223*, L223I, L223V, L228F, L228H, L228T, L239*, L239F, L239T, L250*, L250P, L250T, L44*, L44M, L47C, L47V, L57*, L57X, L60S, L79F, L79S, L84W, L90*, L90V, M240D, M240L, M240R, M240W, N11D, N11K, N124T, N177*, N177T, N229*, N229T, N231D, N231F, N231K, N231L, N231M, N231Q, N231T, N243Q, N243T, N252E, N252T, N61R, N61T, N61Y, N62K, N62M, N67D, N67T, P137L, PDA, P13H, P13L, P13S, P154L, P154R, P154T, P180L, P180S, P185L, P185S, P185V, P194Q, P200A, P200L, P200S, P200T, P201A, P201L, P201R, P201S, P214T, P236L, P236T, P246L, P246Q, P249A, P249F, P249H, P249I, P249X, P30L, P30S, P42L, P83A, Q103K, Q135S, Q136N, Q157R, Q157S, Q221A, Q221R, Q248F, Q248L, Q248S, Q254A, Q254K, Q28S, Q53H, Q53K, Q53N, Q74R, Q92H, Q92S, R181H, RMS, R181V, R226H, R226P, R226V, R245A, R253G, R253Q, R27A, R58G, R89D, R89F, R89I, R89X, R89Y, S105L, S105Q, S151A, S151I, S151Q, S165F, S165P, S172E, S172V, S187I, S187P, S196H, S196L, S216A, S216Q, S218A, S218Q, S219A, S219Q, S258F, S258P, S29C, S29P, S43P, S43T, S48L, S50P, S56F, S56N, S56P, S56X, S73L, S73N, S73X, S99H, T108L, T125I, T125P, T168K, T168N, T168Q, T176H, T176L, T192D, T192F, T192I, T192N, T192P, T192X, T198D, T198I, T198P, T199A, T199H, T199P, T207D, T207I, T207P, T207S, T35I, T35L, T37Q, T55L, T87L, V109M, V109W, V121F, V134C, V134F, V142F, V149G, V149L, V159L, V159S, V160C, V160L, V162A, V162C, V206*, V206C, V206M, V210C, V217L, V217R, V217S, V222A, V222C, V222G, V241G, V241W, V241X, V31L, V49F, V68L, V68W, V78C, W123G, W123R, W16G, W191*, W191G, WML, W208G, W208L, W208S, W244*, W244G, W244L, W97C, W97G, Y114H, Y114M, Y127M, Y190*, Y190L, Y190T, Y193C, Y193F, Y193I, Y193L, Y193T, Y193V, Y193X, Y40M, Y51F, Y51M, Y51T, Y51X, Y88T, K9N, and S29A.

Exemplary stabilizing agents of CA2-derived DDs include Celecoxib (Celebrex), Valdecoxib, Rofecoxib (Vioxx), Acetazolamide, Methazolamide, Dorzolamide, Brinzolamide, Diclofenamide, Ethoxzolamide, Zonisamide, Dansylamide, and Dichlorphenamide.

Exemplary PPAR gamma-derived DDs are described in WO 2018/160993 A1 and US 2022/0213449 A1, the contents of which are incorporated herein in their entireties. An amino acid sequence of full-length wild-type human PPAR gamma, corresponding to the GenBank Access NO. P37231, is as follows:

(SEQ ID NO: 10)
MGETLGDSPIDPESDSFTDTLSANISQEMTMVDTEMPFWPTNFGISSVDLSVMEDHS
HSFDIKPFTTVDESSISTPHYEDIPFTRTDPVVADYKYDLKLQEYQSAIKVEPASPP
YYSEKTQLYNKPHEEPSNSLMAIECRVCGDKASGFHYGVHACEGCKGFFRRTIRLKL
IYDRCDLNCRIHKKSRNKCQYCRFQKCLAVGMSHNAIRFGRMPQAEKEKLLAEISSD
IDQLNPESADLRALAKHLYDSYIKSFPLTKAKARAILTGKTTDKSPFVIYDMNSLMM
GEDKIKFKHITPLQEQSKEVAIRIFQGCQFRSVEAVQEITEYAKSIPGFVNLDLNDQ
VTLLKYGVHEIIYTMLASLMNKDGVLISEGQGFMTREFLKSLRKPFGDFMEPKFEFA
VKFNALELDDSDLAIFIAVIILSGDRPGLLNVKPIEDIQDNLLQALELQLKLNHPES
SQLFAKLLQKMTDLRQIVTEHVOLLQVIKKTETDMSLHPLLQEIYKDLY

A truncated PPAR gamma sequence that corresponds to the ligand binding domain of PPAR gamma, comprising the sequence of amino acids 317 to 505 of SEQ ID NO:10, is as follows:

(SEQ ID NO: 11)
SVEAVQEITEYAKSIPGFVNLDLNDQVTLLKYGVHEIIYTMLASLMNKDGVLISEGQ
GFMTREFLKSLRKPFGDFMEPKFEFAVKFNALELDDSDLAIFIAVIILSGDRPGLLN
VKPIEDIQDNLLQALELQLKLNHPESSQLFAKLLQKMTDLRQIVTEHVQLLQVIKKT
ETDMSLHPLLQEIYKDLY

A PPAR gamma-derived DD can comprise a full-length wild-type PPAR gamma sequence or can comprise one or more amino acid substitutions and/or truncations at the N and/or C-terminal end. In some embodiments, a PPAR gamma-derived DD comprises a truncated wild-type PPAR gamma sequence. For example, a PPAR gamma-derived DD sequence can be at least 80%, at least 85%, at least 90%, at least 95%, at least 96%, at least 97%, at least 98%, at least 99% or 100% identical to SEQ ID NO:10 or SEQ ID NO:11.

Exemplary stabilizing agents of PPAR gamma-derived DDs include Pioglitazone and Posiglitazone.

Exemplary NQO2-derived DDs are described in WO 2018/160993 A1 and US 2022/0213449 A1, the contents of which are incorporated herein in their entireties. An amino acid sequence of wild-type human NQO2, corresponding to the GenBank Access NO. P16083, is as follows:

(SEQ ID NO: 12)
MAGKKVLIVYAHQEPKSFNGSLKNVAVDELSRQGCTVTVSDLYAMNLEPRATDKDIT
GTLSNPEVENYGVETHEAYKQRSLASDITDEQKKVREADLVIFQFPLYWFSVPAILK
GWMDRVLCQGFAFDIPGFYDSGLLQGKLALLSVTTGGTAEMYTKTGVNGDSRYFLWP
LQHGTLHFCGFKVLAPQISFAPEIASEEERKGMVAAWSQRLQTIWKEEPIPCTAHWH
FGQ

A NQO2-derived DD can comprise a wild-type NQO2 sequence or can comprise one or more amino acid substitutions and/or truncations at the N and/or C terminal end. For example, a NQO2-derived DD sequence can be at least 80%, at least 85%, at least 90%, at least 95%, at least 96%, at least 97%, at least 98%, at least 99% or 100% identical to SEQ ID NO:12.

Exemplary stabilizing agents of NQO2-derived DDs include Imatinib and Melatonin.

Exemplary estrogen receptor ligand binding domain (ERLBD)-derived DDs are described by Miyazaki et al. (Miyazaki et al., 2012, J Am Chem Soc. 134(9): 3942-3945) and in US 2014/0255361 A1, the contents of which are incorporated herein in its entirety. An amino acid sequence of wild-type human ERLBD, corresponding to residues 305-549 of human estrogen receptor 1 (ERS1) of GenBank Access NO AA128574.1, is as follows:

(SEQ ID NO: 13)
SLALSLTADQMVSALLDAEPPILYSEYDPTRPFSEASMMGLLTNLADRELVHMINWA
KRVPGFVDLTLHDQVHLLECAWLEILMIGLVWRSMEHPGKLLFAPNLLLDRNQGKCV
EGMVEIFDMLLATSSRFRMMNLQGEEFVCLKSIILLNSGVYTFLSSTLKSLEEKDHI
HRVLDKITDTLIHLMAKAGLTLQQQHQRLAQLLLILSHIRHMSNKGMEHLYSMKCKN
VVPLYDLLLEMLDAHRL

An ERLBD-derived DD can comprise a wild-type ERLBD sequence or can comprise one or more amino acid substitutions and/or truncations at the N and/or C terminal end. For example, an ERLBD-derived DD sequence can be at least 80%, at least 85%, at least 90%, at least 95%, at least 96%, at least 97%, at least 98%, at least 99% or 100% identical to SEQ ID NO:13. Exemplary amino acid substitutions and combinations that can be included in an ERLBD DD include T371A, L384M, M421G, N519S, G521R, and Y537S. Combinations of the foregoing substitutions can also be used. In some embodiments, the DD comprises an amino acid sequence which is identical to SEQ ID NO:13 except for a T371A, L384M, M421G, N519S, G521R, or Y537S substitution, or a combination thereof.

Exemplary stabilizing agents of ERLBD-derived DDs include CMP8, 4-hydroxytamoxifen (afimoxifene), tamoxifen, fulvestrant, and raloxifene.

Exemplary DDs derived from the fluorescent protein, UnaG, are described by Navarro et al. (Navarro et al., 2016, ACS Chem Biol. 11(8):2101-4). An amino acid sequence of wild-type UnaG, corresponding to the GenBank Access NO. AB763906, is as follows:

(SEQ ID NO: 14)
MVEKFVGTWKIADSHNFGEYLKAIGAPKELSDGGDATTPTLYISQKDGDKMTVKIEN
GPPTFLDTQVKFKLGEEFDEFPSDRRKGVKSVVNLVGEKLVYVQKWDGKETTYVREI
KDGKLVVTLTMGDVVAVRSYRRATE

An UnaG-derived DD can comprise a wild-type UnaG sequence or can comprise one or more amino acid substitutions and/or truncations at the N and/or C terminal end. For example, an UnaG-derived DD sequence can be at least 80%, at least 85%, at least 90%, at least 95%, at least 96%, at least 97%, at least 98%, at least 99% or 100% identical to SEQ ID NO:14. Exemplary amino acid substitutions and combinations that can be included in an UnaG DD include A36V and R136G. Combinations of the foregoing substitutions can also be used. In some embodiments, the DD comprises an amino acid sequence which is identical to SEQ ID NO:14 except for a A36V or R136G substitution, or a combination thereof.

An exemplary stabilizing agent of UnaG-derived DDs is bilirubin.

### 6.2.2. Translational Separator

The gene transfer constructs of the disclosure comprise a translational separator, located between the sequence encoding the DD and the sequence encoding a peptide of interest. In some embodiments, wherein the gene transfer construct comprises a peptide of interest sequence encoding more than one copy of one or more peptides of interest, each copy is separated by a translational separator. A translational separator is a sequence that encodes a self-cleaving peptide, which (without being bound by theory) can allow for efficient, stoichiometric, concordant expression of discrete peptide products from a single mRNA molecule through ribosomal skipping.

The term “self-cleaving”, as used in the art, is not entirely accurate, and is not intended to imply proteolytic cleavage. Without being bound by theory, self-cleaving peptides are thought to function by inducing the ribosome to skip the synthesis of a peptide bond at their C-termini. This peptide bond skipping leads to separation between, for example, the end of the self-cleaving peptide sequence and the next peptide downstream, without preventing downstream translation.

First discovered in picornaviruses, self-cleaving peptides are short peptides, typically between 18 to 22 amino acids in length. Using self-cleaving peptides, picornaviruses and many other viruses are capable of producing equimolar levels of multiple genes from the same mRNA molecule.

Exemplary self-cleaving peptides suitable for use as translational separators in the gene transfer constructs of the disclosure include 2A peptides, which have the consensus motif DVEXNPGP (SEQ ID NO:15), where X is any amino acid. The translational separation via a 2A peptide typically occurs between the glycine and proline residues found on the C-terminus of the resulting peptide, meaning the upstream cistron will have a few additional residues added to the end, while the downstream cistron will start with the proline residue. Any 2A peptide may be chosen to be used as translational separator, e.g., a P2A peptide, an E2A peptide, an F2A peptide, a T2A peptide, or any other 2A peptide described in Szymczak-Workman et al., 2012, Cold Spring Harb Protoc doi:10.1101/pdb.ip067876 or Wang & Marchisio, 2021, Synth Syst Biotechnol 6:254-261. The polynucleotide sequence encoding a 2A peptide may be codon optimized or otherwise modified by the methods known in the art. For instance, an optional N-terminal GSG sequence may be included to increase the efficiency of translational separation.

In some embodiments, the translational separator encodes a P2A peptide, or fragments or variants thereof. An exemplary amino acid sequence of a P2A peptide is ATNFSLLKQAGDVEENPGP (SEQ ID NO:16). An exemplary amino acid sequence of a P2A peptide with a N-terminal GSG is GSGATNFSLLKQAGDVEENPGP (SEQ ID NO:17).

In some embodiments the translational separator encodes an E2A peptide, or fragments or variants thereof. An exemplary amino acid sequence of an E2A peptide is QCTNYALLKLAGDVESNPGP (SEQ ID NO:18). An exemplary amino acid sequence of a E2A peptide with a N-terminal GSG is GSGQCTNYALLKLAGDVESNPGP (SEQ ID NO:19).

In some embodiments the translational separator encodes an F2A peptide, or fragments or variants thereof. An exemplary amino acid sequence of an F2A peptide is VKQTLNFDLLKLAGDVESNPGP (SEQ ID NO:20). The amino acid sequence of an F2A peptide with a N-terminal GSG is GSGVKQTLNFDLLKLAGDVESNPGP (SEQ ID NO:21).

In some other embodiments the translational separator encodes a T2A peptide, or fragments or variants thereof. An exemplary amino acid sequence of a T2A peptide is EGRGSLLTCGDVEENPGP (SEQ ID NO:22). An exemplary amino acid sequence of a T2A peptide with a N-terminal GSG is GSGEGRGSLLTCGDVEENPGP (SEQ ID NO:23).

### 6.2.3. Peptide of Interest

The gene transfer constructs of the disclosure comprise a sequence encoding one or more copies of one or more peptides of interest (POIs). Gene transfer constructs comprising two or more POI encoding sequences can comprise (1) two or more identical sequences encoding the same POI, linked to one another via a translational separator; (2) two or more different sequences encoding the same POI, linked to one another via a translational separator; (3) two or more different sequences encoding two or more distinct POIs, linked to one another via a translational separator; or (4) any combination of sequences described in (1) to (3), wherein POI encoding sequences are linked to one another via translational separators.

In some embodiments, the gene transfer construct comprises one POI encoding sequence. In some embodiments, the gene transfer construct comprises two or more POI encoding sequences.

In some embodiments, the gene transfer construct comprises two identical sequences encoding a POI that are linked via a translational separator. In some other embodiments, the gene transfer construct comprises two different sequences encoding the same POI that are linked via a translational separator. In further embodiments, the gene transfer construct comprises two different sequences encoding two different POIs that are linked via a translational separator.

In some embodiments, the gene transfer construct comprises three or more POI sequences, wherein each POI is linked to one another via a translational separator. In some embodiments, the gene transfer construct comprises three or more identical POI sequences. In some embodiments, gene transfer construct comprises three or more different POI sequences, which encode the same POI. In some embodiments, the gene transfer construct comprises three or more different POI sequences, which encode the same POI. In some embodiments, the gene transfer construct comprises three or more different POI sequences, which encode distinct POIs. In some other embodiments, the gene transfer construct comprises three or more POI sequences, wherein some of which encode the same POI.

POIs are typically less than 150 amino acids, e.g., no more than 130, 100, 80, 60, 40, 20, or 10 amino acids in length. In some embodiments, a POI is at least 2 amino acids, at least 3 amino acids, at least 4 amino acids, at least 5 amino acids, at least 7 amino acids, at least 10 amino acids, or at least 20 amino acids in length, but no more than 150 amino acids in length. In some embodiments, a POI is no more than 130, no more than 100, no more than 80, no more than 60, no more than 50, no more than 40, no more than 20, or no more than 10 amino acids in length. In some embodiments, a POI has a length within a range bounded by any two values set forth in this paragraph, e.g., 3 to 100, 4 to 50, 5 to 20, or 6 to 10 amino acids in length.

In one aspect, the POIs of the disclosure are peptides that can be used as PPI inhibitors. Nonlimiting examples of PPI inhibitor peptides that can be included in the gene transfer constructs of the disclosure include peptides that inhibit dynamin-1-related protein (Drp1), T-lymphokine-activated killer cell-originated protein kinase (TOPK), Sal-like protein 4 (SALL4), Ras, p53, protein phosphatase 2A (PP2A), signal transducer and activator of transcription 3 (STAT3), Yes-associated protein (YAP), Bcl-2 family proteins (e.g., anti-apoptotic Bcl-2 family proteins), NOTCH, estrogen receptors, microtubule-associated protein light chain 3 (LC3), or E3 ligase MDM2/MDMX.

Drp1 is a cytosolic protein, which in response to various stimuli, translocates to the surface of mitochondria, where it mediates mitochondrial fission (Chan, 2006, Cell 125:1241-1252; Chang and Blackstone, 2010; Ann NY Acad Sci, 1201:34-39). Drp1 is thought to wrap around the mitochondria to induce fission powered by its GTPase activity (Smirnova et al., 2001, Mol Biol Cell 12:2245-2256). Cell culture studies demonstrated that Drp1-induced excessive mitochondrial fission and fragmentation and plays an active role in apoptosis (Frank et al., 2001, Dev Cell, 1:515-525; Estaquier and Arnoult, 2007, Cell Death Differ, 14:1086-1094), autophagic cell death (Twig et al., 2008, EMBO J 27:433-446; Barsoum et al., 2006, EMBO J 25:3900-3911) and necrosis (Wang et al., 2012, Cell 148:228-243). Inhibition of Drp1 leads to decreased mitochondrial fragmentation.

In some embodiments, gene transfer constructs of the disclosure comprise one or more POI sequences encoding a Drp1 inhibitor peptide. Exemplary Drp1 inhibitor peptides are described in Qi et al., 2013. J Cell Sci 126(3):789-802 and U.S. Pat. No. 10,912,815, the contents of which are incorporated herein in their entirety. Exemplary amino acid sequences of Dp1 inhibitor peptides are set forth in Table 2.

In certain embodiments, gene transfer constructs of the disclosure comprise one or more POI sequences encoding the Drp1 inhibitor peptide, P110, which has the amino acid sequence DLLPRGT (SEQ ID NO:24) and the exemplary nucleotide sequence GATCTGCTGCCACGCGGGACG (SEQ ID NO:25), or a variant of P110, e.g., P110 variant a, which has the amino acid sequence DLLPRGS (SEQ ID NO:26).

In other embodiments, gene transfer constructs of the disclosure comprise one or more POI sequences encoding a Drp1 inhibitor peptide selected from the group consisting of: P108, P109, P111, P112, and P113. The amino acid sequences of P108, P109, P111, P112 and P113 are set forth as SEQ ID NOs:27-31, respectively, in Table 2.

TOPK, also known as PDZ-binding kinase or PBK is a protein upregulated in various actively proliferative cells and has been linked to mitotic progression and tumor cellular proliferation (Han et al., 2021 Cells 10(2):371; Matsuo et al., 2014. Sci Transl Med, 6(259): 259ra145). Inhibition of TOPK has been shown to decrease tumor growth (Kim et al., 2012. Cancer Res 72:3060-8).

In some embodiments, a gene transfer construct of the disclosure encodes one or more sequences that encode a peptide inhibitor of TOPK. An exemplary amino acid sequence of a peptide inhibitor of TOPK is MEGISNFKTPSKLSEKKK (SEQ ID NO:32) and presented in Table 2.

SALL4 encodes a zinc-finger transcription factor that is expressed during early development and its expression is undetectable in most adult tissues. However, SALL4 is expressed in various cancers, including liver, lung, ovarian, endometrial, and breast cancers, as well as acute myeloid leukemia (Jones, 2013. Nat Rev Clin Oncol 10(426)).

In some embodiments, a gene transfer construct of the disclosure comprises one or more sequences that encode a peptide inhibitor of SALL4. An exemplary amino acid sequence of a peptide inhibitor of SALL4 is MSRRKQAKPQHI (SEQ ID NO:33) and presented in Table 2.

The Ras family represents some of the earliest described oncogenes. Unregulated activity of Ras gene products has been implicated in a wide variety of cancers. Indeed, Ras alterations have been found to contribute to 20-30% of all human cancers (Gimple & Wang 2019. Front Oncol 9:965 doi:10.3389/fonc.2019.00965) and Ras mutations have been found in nearly 100% of pancreatic cancer tumors (O'Bryan, 2019. Pharmacol Res 139:503-511).

In some embodiments, a gene transfer construct of the disclosure comprises one or more sequences that encode a peptide inhibitor of Ras. An exemplary amino acid sequence of a peptide inhibitor of Ras is HYPWFKARLYPL (SEQ ID NO:34) and presented in Table 2.

p53 is a transcription factor that functions as a tumor suppressor (Surget S et al., OncoTargets and Therapy 7: 57-68, 2013). The gene encoding p53 has been found to be mutated in approximately half of all human cancers (Marei et al., 2021 Cancer Cell Int 703 doi.org/10.1186/s12935-021-02396-8).

In some embodiments, a gene transfer construct of the disclosure comprises one or more sequences that encode a peptide inhibitor of p53. Exemplary amino acid sequences of p53 peptide inhibitors are presented in Table 2 and include SEQ ID NOs:35-40.

PP2A is a serine/threonine phosphatase that modulates the activity of proteins in several oncogenic signaling cascades (Kurimchak & Graña, 2015. Cell Cycle 14:18-30).

In some embodiments, a gene transfer construct of the disclosure comprises one or more sequences that encode a peptide inhibitor of PP2A. Exemplary amino acid sequences of PP2A peptide inhibitors are presented in Table 2 and include SEQ ID NOs:41-44.

STAT3 is a transcription factor, and alterations in its activity, such as loss of function, gain of function, or constitutive activation, are associated with recurrent infections, disordered bone and tooth development, auto-immune diseases, and various cancers (see, e.g., Levy et al., 2007. New Eng J Medi 357:1655-1658; Milner et al., 2015. Blood 125: 591-9; Klampfer, 2006. Curr Cancer Drug Targ 6:107-121; Alvarez et al., 2006. Cancer Research 66: 3162-3168; Yin et al., 2006. Mol Cancer 5:15. Doi:10.1186/1476-4598-5-15; Kusaba et al., 2006. Onc Rep 15:1445-51. Doi:10.3892/or.15.6.1445).

In some embodiments, a gene transfer construct of the disclosure comprises one or more sequences that encode a peptide inhibitor of STAT3. An exemplary amino acid sequence of a STAT3 peptide inhibitor has the sequence PLTAVFWLIYVLAKALVTVC (SEQ ID NO:45) and is presented in Table 2.

YAP is a transcription factor regulated by the Hippo pathway, and alterations in its activity, such as gain in function, are associated with various cancers (Yu, et al., 2015. Cell. 163(4):811-28; Yimlamai et al., 2015. J Hepatol. 63(6):1491-501). The Hippo pathway controls several cell functions central to tumorigenesis, e.g., cell proliferation and apoptosis, and is deregulated in several human cancers. When the Hippo pathway is on, YAP is degraded and a VGLL family member (including VGLL1-4) binds to TEAD1-TEAD4, downregulating downstream genes. When the Hippo pathway is off, YAP binds to TEAD1-TEAD4, inducing transcription of downstream genes.

In some embodiments, a gene transfer construct of the disclosure comprises one or more sequences that encode a peptide inhibitor of YAP. In some embodiments, the peptide inhibitor of YAP comprises sequence fragments from both VGLL4 and YAP. In some embodiments, the peptide inhibitor of YAP comprises sequence fragments of TEADs binding regions from VGLL4 and YAP. In other embodiments, the peptide inhibitor of YAP comprises a Super-TDU as described in Jiao et al., 2015. Cancer Cell, 25: 66-180 and WO 2017/127750, the contents of which are incorporated herein in their entirety. Exemplary amino acid sequences of YAP peptide inhibitors are presented in Table 2 and include SEQ ID NOs:46-56.

Bcl-2 family members are important regulators of apoptosis. Without wishing to be bound by theory, it is known in the art that the balance of pro-apoptotic Bcl-2 family proteins and anti-apoptotic Bcl-2 family proteins in a cell is important for regulation of apoptosis. Anti-apoptotic Bcl-2 family proteins, such as Bxl-2, Bcl-xL, and Mci-1, are expressed in a wide range of tumors and their inhibition in cancer cells leads to apoptosis, including in cancer cells that are resistant to conventional chemotherapies. Four Bcl-2 homology domains (BH1-BH4) share a folding motif that creates a hydrophobic groove, which mediates binding to an α-helical stretch protein with BH3 domains, called BH3-only proteins (Pelay-Gimeno et al., 2015, Angew Chem Int Ed Engl 54, 8896-8927). Several BH3 domain mimetics have been developed to target anti-apototic Bcl-2 family proteins (D'Aguanno and Del Bufalo, 2020. Cells 9(5):1287). Structural studies have shown that the BH3 domain of BH3-only proteins can bind as an amphipathic helix in a surface-exposed hydrophobic groove of an anti-apoptotic Bcl-2 family member (Day et al., 2008. J. Mol. Biol. 380:958-971). Exemplary BH3 domains were described in WO 2017/127750, the contents of which are incorporated herein in their entirety.

In some embodiments, the BH3 peptide is a human BH3 domain. In other embodiments, the BH3 peptide may be from a non-human species, e.g., Caenorhabditis elegans, rodents, non-human primates, or any other species.

Typically, a BH3 peptide is derived from a pro-apoptotic Bcl-2 family member, including from an effector pro-apoptotic Bcl-2 family member (e.g., BAK or BAX) or from a BH3-only family member (e.g., BID, BIM, BAD, BIK, BMF, bNIP3, HRK, Noxa, and PUMA). In some embodiments, the BH3 peptide is derived from a BH3-only family member.

In some embodiments, a BH3 peptide may directly bind to a Bcl-2 family protein. For example, in some embodiments, a BH3 peptide may directly bind to a pro-apoptotic Bcl-2 family protein. In some embodiments, the pro-apoptotic Bcl-2 family protein is Bax and/or Bak. In some embodiments, a BH3 domain may directly interact with an anti-apoptotic Bcl-2 family protein. In some embodiments, the anti-apoptotic Bcl-2 family protein may BCL-2, BCL-XL, BCL-w, MCL-1 or BCL2-related protein A1 (BCL2A1).

Exemplary amino acid sequences of BH3 peptides are presented in Table 2 and include SEQ ID NOs: 57-82.

NOTCH signaling is an evolutionary conserved pathway that regulates cell-fate decisions at an individual cell level and aberrant NOTCH signaling has been implicated in the onset and progression of various types of cancer (Pelay-Gimeno et al., 2015, Angew Chem Int Ed Engl 54, 8896-8927; Moellering et al., 2010, Nature, 462(7270): 182-188).

In some embodiments, a gene transfer construct of the disclosure comprises one or more sequences that encode a peptide inhibitor of NOTCH. An exemplary amino acid sequence of a NOTCH peptide inhibitor is presented in Table 2 and includes SEQ ID NO: 83.

Estrogen receptors are hormone-activated transcription factors that are regulated by coactivator proteins. The hyperactivation of estrogen receptors (e.g., ERa) has been implicated in several diseases, including several types of cancer (Pelay-Gimeno et al., 2015, Angew Chem Int Ed Engl 54, 8896-8927; Darnell, 2002, Nat. Rev. Cancer 2:740-749).

In some embodiments, a gene transfer construct of the disclosure comprises one or more sequences that encode a peptide inhibitor of ERα. An exemplary amino acid sequence of an ERα peptide inhibitor is presented in Table 2 and includes SEQ ID NO: 84.

Microtubule-associated protein light chain 3 (LC3) is a protein essential for the maturation of autophagosomes and shares structural homology with ubiquitin. Preclinical studies indicate that the inhibition of LC3-mediated autophagy can improve cancer treatment outcomes (Yang et al., 2022, Front Oncol. (12) doi.org/10.3389/fonc.2022.992171; Gray et al., 2021, Chem Sci, 12(10):3526-3543).

In some embodiments, a gene transfer construct of the disclosure comprises one or more sequences that encode a peptide inhibitor of LC3. An exemplary amino acid sequence of an LC3 peptide inhibitor has the sequence MFPHRVTAZK (SEQ ID NO:85) and is presented in Table 2.

E3 ligase MDM2 and its homolog MDMX regulate the tumor suppressor p53 by binding to its N-terminal transactivation domain, which results in the ubiquitination and degradation of p53. Upregulation of MDM2/MDMX has been detected in various cancers and strategies that inhibit MDM2/MDMX can restore p53 function (Yang et al., 2022, Front Oncol. (12) doi.org/10.3389/fonc.2022.992171; Pelay-Gimeno et al., 2015, Angew Chem Int Ed Engl 54, 8896-8927; Phan et al., 2010, J Biol Chem 285(3): 2174-2183; Pazgier et al., 2009, PNAS, 106(12):4665-70).

In some embodiments, a gene transfer construct of the disclosure comprises one or more sequences that encode a peptide inhibitor of MDM2/MDMX. Exemplary amino acid sequences of MDM2/MDMX peptide inhibitors are presented in Table 2 and include SEQ ID NOs: 86 and 87.

TABLE 2

| POI Name and/or | | SEQ ID |
|---|---|---|
| Function | Sequence | NO: |
| P110 | DLLPRGT | 24 |
| P110 variant a | DLLPRGS | 26 |
| P108 | STQELLRFPK | 27 |
| P109 | KLSAREQRD | 28 |
| P111 | CSVEDLLKFEK | 29 |
| P112 | KGSKEEQRD | 30 |
| P113 | ELLPKGS | 31 |
| TOPK inhibitor | MEGISNFKTPSKLSEKKK | 32 |
| peptide | | |
| SALL4 inhibitor | MSRRKQAKPQHI | 33 |
| peptide | | |
| Ras inhibitor | HYPWFKARLYPL | 34 |
| peptide | | |
| p53 inhibitor | ETFSDLWKLLPE | 35 |
| peptide 1 | | |
| p53 inhibitor | TSFAEYWNLLSP | 36 |
| peptide 2 | | |
| p53 inhibitor | ETFEHWWSQLLS | 37 |
| peptide 3 | | |
| p53 inhibitor | ETFEHWWAQLTS | 38 |
| peptide 4 | | |
| p53 inhibitor | LTFEHSWAQLTS | 39 |
| peptide 5 | | |
| p53 inhibitor | LTFEHWWAQLTS | 40 |
| peptide 6 | | |
| PP2aB56alpha | TPDYFL | 41 |
| PP2aDP7 | VKKKKIKREIKIFRGRSRFRGRSR | 42 |
| PP2aDPT2 | RQKRLIRQKRLIRQKRLI | 43 |
| PP2aDPT5 | VKKKKIKREIKIPRRPGPTRKHYQPYA | 44 |
| STAT3 peptide | PLTAVFWLIYVLAKALVTVC | 45 |
| inhibitor | | |
| Super TDU | SVDDHFAKSLGDTWLQIGGSGNPKTANVPQTVPMRLRK | 46 |
| | LPDSFFKPPE | |
| Super TDU | SVDDHFAKSLGDTWLQIGGSGNPKTANVPQTVPARLRK | 47 |
| (MF2A) | LPDSAFKPPE | |
| Super TDU | SVDDAAAKSLGDTWLQIGGSGNPKTANVPQTVPARLRK | 48 |
| (HFMF4A) | LPDSAFKPPE | |
| Human VGLL4- | DPVVEEHFRRSLGKNYKEPEPAPNSVSITGSVDDHFAK | 49 |
| TDU 1-2 | ALGDTWLQIKAAKD | |
| VGLL4-TDU 1 | DPVVEEHFRRSLGKNYK | 50 |
| (V1) | | |
| VGLL4-TDU 1 | DPVVEEHFRRSLGKNYKE | 51 |
| (V2) | | |
| VGLL4-TDU 1 | DPVVEEHFRRSLGKNYKEPE | 52 |
| (V2) | | |
| VGLL4-TDU 2 | SVSITGSVDDHFAKALGDTWLQIK | 53 |
| (V1) | | |
| VGLL4-TDU 2 | SVSITGSVDDHFAKALGDTWLQIKA | 54 |
| (V2) | | |
| VGLL4-TDU 2 | SVSITGSVDDHFAKALGDTWLQIKAAKD | 55 |
| (V3) | | |
| VGLL4 Super | SVDDHFAKSLGDTWLQI | 56 |
| TDU | | |
| BH3 peptide (BIK) | LALRLACIGDEMDVS | 57 |
| BH3 peptide | EKAELLQGGDLLRQR | 58 |
| (BNIP1) | | |
| BH3 peptide | VESILKKNSDWIWDW | 59 |
| (BNIP3) | | |
| BH3 peptide | EVEALKKSADWVSDW | 60 |
| (BNIP3L) | | |
| BH3 peptide | TAARLKALGDELHQR | 61 |
| (HRK) | | |
| BH3 peptide | IAQELRRIGDEFNAY | 62 |
| (BIMe1) | | |
| BH3 peptide | YGRELRRMSDEFVDS | 63 |
| (BAD) | | |
| BH3 peptide | IARHLAQVGDSMDRS | 64 |
| (BID) | | |
| BH3 peptide | IARKLQCIADQFHRL | 65 |
| (BMF) | | |
| BH3 peptide | CATQLRRFGDKLNFR | 66 |
| (NOXA) | | |
| BH3 peptide | IGAQLRRMADDLNAQ | 67 |
| (PUMA) | | |
| BH3 peptide | LSRRLKVTGDLFDIM | 68 |
| (Beclin1) | | |
| BH3 peptide | IVELLKYSGDQLERK | 69 |
| (BCL-Gs) | | |
| BH3 peptide | ALETLRRVGDGVQRN | 70 |
| (MCL-1S) | | |
| BH3 peptide | IGSKLAAMCDDFDAQ | 71 |
| (EGL-1) | | |
| BH3 peptide | IGRKLTVMCDEFDSE | 72 |
| (CED-13) | | |
| BH3 peptide | NIRRLRALADGVQKV | 73 |
| (ApoL1) | | |
| BH3 peptide | NIDKLRALADDIDKT | 74 |
| (ApoL6) | | |
| BH3 peptide | MVTLLPIEGQEIHFF | 75 |
| (HER2) | | |
| BH3 peptide | PTVPLPSETDGYVAP | 76 |
| (HER4) | | |
| BH3 peptide | PQRYLVIQGDDRMKL | 77 |
| (MAP1) | | |
| BH3 peptide | TVGELSRALGHENGS | 78 |
| (MULE) | | |
| BH3 peptide (BIK) | VGQLLQDMGDDVYQQ | 79 |
| BH3 peptide | LHEVLNGLLDRPDWE | 80 |
| (SPHK2) | | |
| BH3 peptide | AVHSLSRIGDELYLE | 81 |
| (RAD9) | | |
| BH3 peptide | NPKFLKNAGRDCSRR | 82 |
| (TGM2) | | |
| MAML1(21-36) | ERLRRRIELCRRHHST | 83 |
| PERM-1 | KCILCRLLQ | 84 |
| SUPR4B | MFPHRVTAZK | 85 |
| pDI | LTFEHYWAQLTS | 86 |
| PMI | TSFAEYWNLLSP | 87 |

### 6.3. Nucleic Acids and Host Cells

### 6.3.1. Nucleic Acids

The present disclosure provides nucleic acid gene transfer constructs, e.g., as described in Section 6.2. The nucleic acid can be, for example, an mRNA molecule as described in Section 6.3.1.1, a modified mRNA molecule as described in Section 6.3.1.2, or a DNA molecule, e.g., an expression plasmid or vector as described in Section 6.3.1.3.

6.3.1.1. mRNA

In one aspect, the present disclosure provides gene transfer constructs in an mRNA form.

An mRNA molecule of the disclosure may comprise naturally occurring nucleobases, nucleosides, or nucleotides as well as non-naturally occurring nucleobases, nucleosides, or nucleotides.

An mRNA may include a 5′ untranslated region (5′-UTR), a 3′ untranslated region (3′-UTR), and/or a coding region (e.g., an open reading frame). An mRNA may include any suitable number of bases, including tens (e.g., 10, 20, 30, 40, 50, 60, 70, 80, 90 or 100), hundreds (e.g., 200, 300, 400, 500, 600, 700, 800, or 900) or thousands (e.g., 1000, 2000, 3000, 4000, 5000, 6000, 7000, 8000, 9000, 10,000) of bases.

In some embodiments, an mRNA molecule as described herein may include a 5′ cap structure, a chain terminating nucleotide, optionally a Kozak sequence (also known as a Kozak consensus sequence), a stem loop, a polyA sequence, and/or a polyadenylation signal.

In some embodiments, the mRNA sequence is codon optimized. Codon optimization methods are known in the art and may be useful for a variety of purposes including: matching codon frequencies in host organisms to ensure proper folding, bias GC content to increase mRNA stability or reduce secondary structures, minimize tandem repeat codons or base runs that may impair gene construction or expression, customize transcriptional and translational control regions, insert or remove proteins trafficking sequences, remove/add posttranslational modification sites in encoded proteins (e.g., glycosylation sites), add, remove or shuffle protein domains, insert or delete restriction sites, modify ribosome binding sites and mRNA degradation sites, adjust translation rates to allow the various domains of the protein to fold properly, or to reduce or eliminate problem secondary structures within the polynucleotide.

In some embodiments, a polynucleotide (e.g., an mRNA) of the disclosure comprises a sequence-optimized nucleotide sequence (e.g., an ORF) encoding a peptide (e.g., P110 peptide), a functional fragment, or a variant thereof, wherein the peptide (e.g., P110 peptide), functional fragment, or a variant thereof encoded by the sequence-optimized nucleotide sequence has improved properties (e.g., compared to a P110 peptide, functional fragment, or a variant thereof encoded by a reference nucleotide sequence that is not sequence optimized), e.g., improved properties related to expression efficacy after administration in vivo. Such properties include, but are not limited to, improving nucleic acid stability (e.g., mRNA stability), increasing translation efficacy in the target tissue, reducing the number of truncated products, improving the folding or prevent misfolding of the expressed peptide products, reducing toxicity of the expressed peptide products, reducing the immunogenic and/or inflammatory responses to the expressed peptide products, reducing cell death caused by the expressed peptide products, increasing and/or decreasing peptide aggregation.

In some embodiments, the sequence optimized nucleotide sequence is codon optimized for expression in human subjects, having structural and/or chemical features that avoid one or more of the problems in the art, for example, features which are useful for optimizing formulation and delivery of nucleic acid-based therapeutics while retaining structural and functional integrity; overcoming a threshold of expression; improving expression rates; half-life and/or protein concentrations; optimizing protein localization; and avoiding deleterious bio-responses such as the immune response and/or degradation pathways.

In some embodiments, the desired property of the gene transfer construct of the disclosure is the level of expression of a peptide of interest (e.g., P110) encoded by an optimized sequence disclosed herein. Peptide expression levels can be measured using one or more expression systems. In some embodiments, expression can be measured in cell culture systems, e.g., CHO cells or HEK293 cells. In some embodiments, expression can be measured using in vitro expression systems prepared from extracts of living cells, e.g., rabbit reticulocyte lysates, or in vitro expression systems prepared by assembly of purified individual components. In other embodiments, peptide expression is measured in an in vivo system, e.g., mouse, rabbit, monkey, etc.

In some embodiments, peptide expression in solution form (e.g., a soluble form of a peptide of interest) can be desirable. Therefore, in some embodiments, a reference sequence can be sequence optimized to yield a sequence optimized nucleic acid sequence having optimized levels of expressed peptides in soluble form. Levels of peptide expression and other properties such as solubility, levels of aggregation, and the presence of truncation products (i.e., fragments due to proteolysis, hydrolysis, or defective translation) can be measured according to methods known in the art, for example, using electrophoresis (e.g., native or SDS-PAGE) or chromatographic methods (e.g., HPLC, size exclusion chromatography, etc.).

6.3.1.2. Modified mRNA

In one aspect, the present disclosure provides gene transfer constructs in a modified mRNA form, for example, an mRNA molecule with modified nucleobases, nucleosides, or nucleotides.

In some embodiments, modified mRNAs may have useful properties, including enhanced stability, intracellular retention, enhanced translation, and/or the lack of a substantial induction of the innate immune response of a cell into which the mRNA is introduced, as compared to a reference unmodified mRNA. Therefore, use of modified mRNAs may enhance the efficiency of protein production, intracellular retention of nucleic acids, as well as possess reduced immunogenicity.

Any number (e.g., all, some, or none) of nucleobases, nucleosides, or nucleotides may be an analog of a canonical species, substituted, modified, or otherwise non-naturally occurring. In some embodiments, all of a particular nucleobase type may be modified. In some embodiments, an mRNA includes one or more (e.g., 1, 2, 3 or 4) different modified nucleobases, nucleosides, or nucleotides. In some embodiments, an mRNA includes one or more (e.g., 1, 2, 3, 4, 5, 6, 7, 8, 9, 10, 20, 30, 40, 50, 60, 70, 80, 90, 100, or more) different modified nucleobases, nucleosides, or nucleotides. In some embodiments, the modified mRNA may have reduced degradation in a cell into which the mRNA is introduced, relative to a corresponding unmodified mRNA.

The mmRNA molecules of the disclosure can include a combination of modifications to the sugar groups, nucleobases, and/or the internucleoside linkages. These combinations can include any one or more modifications described herein.

In some embodiments, an mRNA molecule of the disclosure may be modified in a coding region (e.g., an open reading frame encoding one of the domains of a gene transfer construct). In other embodiments, an mRNA may be modified in regions besides a coding region. For example, in some embodiments, a 5′-UTR and/or a 3′-UTR are provided, wherein either or both may independently contain one or more different nucleoside modifications. In some other embodiments, an mRNA molecule may be modified both in the coding and noncoding regions.

Examples of nucleoside modifications and combinations thereof that may be included in mmRNAs of the present disclosure include, but are not limited to, those described in PCT Patent Application Publications: WO 2012/045075, WO 2014/081507, WO 2014/093924, WO 2014/164253, WO 2014/159813, and WO 2017/127750, the contents of each of which are incorporated herein by reference in their entireties.

### 6.3.1.3. Expression Vectors and Plasmids

In another aspect, the present disclosure relates to an expression vector or expression plasmid, comprising or encoding a gene transfer construct of the disclosure, e.g., as described in Section 6.2.

Recombinant expression vectors can comprise a nucleic acid sequence encoding the components of a gene transfer construct in a form suitable for expression in a host cell. Therefore, the recombinant expression vectors include one or more regulatory elements, e.g., a TATA box, a CAAT box, a GC box, a promoter etc. Exemplary promoters include SV40, CMV, PGK1, EF1a, Ubs, TRE, UAS, CaMKIIa, and CAG promoters. A promoter can be selected on the basis of the host cells to be used for expression, that is operably linked to the nucleic acid sequence to be expressed, wherein “operably linked” is intended to mean that the nucleotide sequence of interest is linked to the regulator element(s) in a manner that allows for expression of the nucleotide sequence (e.g., in an in vitro transcription/translation system or in a host cell when the vector is introduced into the host cell).

Mammalian expression vectors can be used to introduce a specific nucleic acid fragment into mammalian systems for mRNA or protein expression. Nonlimiting examples of mammalian expression vectors include the adenoviral vectors, vaccinia vectors, retroviral vectors, the pSV and the pCMV series of plasmid vectors, and baculovirus.

An exemplary mammalian expression vector that can be used to deliver to and express gene transfer constructs in host cells is pCAGGS (Niwa, 1991 Gene, 108 (2):193-200). In some embodiments, the expression vector is pCAGGS.

Other vectors can be used to express the gene transfer constructs of the disclosure in host cells, such as any of the vectors described by Okayama and Berg, 1983 Mol. Cell. Biol. 3:280. Another high expression vector, PMLSV N1/N4 was described by Cosman et al., 1984 Nature 312:768. Additional exemplary mammalian expression vectors are described in WO 2009/102569, the contents of which are incorporated herein in their entirety, which include: pDC406, pFN11 A (BIND) Flexi®, pGL4.31, pFC14A (HaloTag® 7) CMV Flexi®, pFC14K (HaloTag® 7) CMV Flexi®, pFN24A (HaloTag® 7) CMVd3 Flexi®, pFN24K (HaloTag® 7) CMVd3 Flexi®, HaloTag™ pHT2, pACT, pAdVAntage™, pALTER®-MAX, pBIND, pCAT®3-Basic, pCAT®3-Control, pCAT®3-Enhancer, p C AT® 3-Promoter, pCI, pCMVTNT™, pGbluc, pSI, pTARGET™, pTNT™, pF12A RM Flexi®, pF12K RM Flexi®, pReg neo, pYES2/GS, pAckCMW5-DEST Gateway® Vector, pAckPL-DEST™ Gateway® Vector, Gateway® pDEST™27 Vector, Gateway® pEF-DEST51 Vector, Gateway® pcDNA™-DEST47 vector, pCMV/Bsd Vector, pEF6/His A, B, & c, pcDNA™6.2-DEST, pLenti6/TR, pLP-AcGFP1-C, pLPS-AcGFP1-N, pLP-IRESneo, pLP-TRE2, pLP-RevTRE, pLP-LNCX, pLP-CMV-HA, pLP-CMV-Myc, pLP-RetroQ and pLP-CMVneo.

In some embodiments, a mammalian expression vector of the disclosure is a vector based on vaccinia virus; poliovirus; adenovirus (see, e.g., Li et al., 1994, Invest Opthalmol Vis Sci 35:2543-2549; Borras et al, 1999, Gene Ther 6:515-524; Li and Davidson, 1995, PNAS 92:7700-7704; Sakamoto et al., 1999, H Gene Ther 5:1088-1097, 1999; WO 94/12649, WO 93/03769; WO 93/19191; WO 94/28938; WO 95/11984 and WO 95/00655); adeno-associated virus (AAV) (see, e.g., Ali et al., 1998, Hum Gene Ther 9:81 86; Flannery et al., 1997, PNAS 94:6916-6921; Bennett et al., 1997, Invest Opthalmol Vis Sci 38:2857-2863; Jomary et al., 1997, Gene Ther 4:683 690; Rolling et al., 1999, Hum Gene Ther 10:641-648; Ali et al., 1996, Hum Mol Genet 5:591-594; WO 93/09239); SV40; herpes simplex virus; human immunodeficiency virus (see, e.g., Miyoshi et al., 1997, PNAS 94:10319-23; Takahashi et al., 1999, J Virol 73:7812-7816); a retroviral vector (e.g., Murine Leukemia Virus, spleen necrosis virus, and vectors derived from retroviruses such as Rous Sarcoma Virus, Harvey Sarcoma Virus, avian leukosis virus, a lentivirus, human immunodeficiency virus, myeloproliferative sarcoma virus, and mammary tumor virus); and the like. In some embodiments, a recombinant expression vector of the present disclosure is a recombinant lentivirus vector. In some embodiments, a recombinant expression vector of the present disclosure is a recombinant retroviral vector. In some embodiments, a recombinant expression vector of the present disclosure is an AAV vector.

In some embodiments, the vector comprises a retroviral genome. In some embodiments, a vector comprises an AAV genome. Nucleic acids, such as retroviral genomes and AAV genomes, can be provided in the form of a particle, for example a viral particle (e.g., retroviral particle or AAV particle).

### 6.3.2. Host Cells

In another aspect, the disclosure provides host cells comprising a nucleic acid of the disclosure. Host cells can be prokaryotic (e.g., bacterial such as E. coli) or eukaryotic (e.g., a human cell line such as HEK293 or 293T). Host cells can be used, for example, to propagate a nucleic acid such as a retroviral genome or plasmid, or to propagate and package a particle, for example a retroviral particle, or to express a peptide of interest.

In some embodiments, the host cell is an animal cell. In some embodiments, the host cell is a mammalian cell. Nonlimiting examples of animal or mammalian host cells suitable for expressing and producing inhibitory peptides include Chinese hamster ovary cells (CHO), such as CHO-K1 (ATCC CCL-61), DG44 (Chasin et al., 1986, Som. Cell Molec. Genet., 12:555-556; and Kolkekar et al., 1997, Biochemistry, 36:10901-10909), CHO-K1 Tet-On cell line (Clontech), CHO designated ECACC 85050302 (CAMR, Salisbury, Wiltshire, UK), CHO clone 13 (GEIMG, Genova, IT), CHO clone B (GEIMG, Genova, IT), CHO-K1/SF designated ECACC 93061607 (CAMR, Salisbury, Wiltshire, UK), RR-CHOK1 designated ECACC 92052129 (CAMR, Salisbury, Wiltshire, UK), dihydrofolate reductase negative CHO cells (CHO/-DHFR, Urlaub and Chasin, 1980, Proc. Natl. Acad. Sci. USA, 77:4216), and dp12.CHO cells (U.S. Pat. No. 5,721,121); monkey kidney CV1 cells transformed by SV40 (COS cells, COS-7, ATCC CRL-1651); human embryonic kidney cells (e.g., 293 cells, or 293T cells, or 293 cells subcloned for growth in suspension culture, Graham et al., 1977, J. Gen. Virol., 36:59); baby hamster kidney cells (BHK, ATCC CCL-10); monkey kidney cells (CV1, ATCC CCL-70); African green monkey kidney cells (VERO-76, ATCC CRL-1587; VERO, ATCC CCL-81); mouse sertoli cells (TM4, Mather, 1980, Biol. Reprod., 23:243-251); human cervical carcinoma cells (HELA, ATCC CCL-2); canine kidney cells (MDCK, ATCC CCL-34); human lung cells (W138, ATCC CCL-75); human hepatoma cells (HEP-G2, HB 8065); mouse mammary tumor cells (MMT 060562, ATCC CCL-51); buffalo rat liver cells (BRL 3A, ATCC CRL-1442); TRI cells (Mather, 1982, Annals N. Y. Acad. Sci., 383:44-68); MCR 5 cells; FS4 cells.

In some embodiments, the host cell is a human cell, such as a human cell line, e.g., HEK293 cells and HeLa cells. Exemplary cells that can be used in the methods include bone marrow cells, stem cells such as hematopoietic stem cells (HSCs) or mesenchymal stem cells (MSCs), immune cells such as T cells, phagocytes, microglial cells, and macrophages. In some embodiment, the cell is a T cell such as a CD4+ and/or CD8+ T cell. Primary cells obtained from a subject, as well as progeny thereof can be used.

The host cells obtained from a subject can be normal cells (e.g., from a healthy donor) or have dysfunctional mitochondria (e.g., from a subject having a disease or disorder). For example, cells can be from a subject having an age-related disease or disorder, such as an autoimmune disease, a metabolic disease, a genetic disease, cancer, a neurodegenerative disease, or immunosenescence.

In some embodiments, the host cell is a healthy human cell. In some embodiments the host cell is a human cell with dysfunctional mitochondria. In some other embodiments, the host cell is a human cancer cell.

In some embodiments, the cancer cell is a liver cancer cell. In some embodiments, the cancer cell is a colorectal cancer cell. In some embodiments, the cancer cell is a hematopoietic cell. In some embodiments, the cancer cell is a myeloid cell. In some embodiments, the cancer cell is a hematopoietic stem cell (e.g., a hematopoietic stem cell from bone marrow, an erythroid stem cell, a myeloid stem cell, a thrombocytic stem cell).

In some embodiments, the host cell is a transgenic cell that has been genetically modified prior to the introduction of the nucleic acids of the disclosure, e.g., an mRNA, an mmRNA, or an expression plasmid comprising the sequence encoding a gene transfer construct.

In some embodiments, the host cell is an in vitro host cell. In some embodiments, the host cell is an in vivo host cell. In some embodiments, the host cell is part of a multicellular organism, for example a mammal, e.g., human.

6.3.3. Methods of Introducing Nucleic Acids into Host Cells

In one aspect, the disclosure provides methods of introducing nucleic acids into host cells in vitro and in vivo.

The methods of introducing nucleic acids into host cells are known in the art. For instance, a nucleic acid (e.g., an mRNA, mmRNA, or expression vector) can be introduced to a host cell by transfection, electroporation, lipofection, injection, via a carrier or delivery agent, or any other means known in the art for delivering nucleic acids to cells.

Transfection is the process of introducing nucleic acids into eukaryotic cells by nonviral methods, e.g., by using various chemical or physical methods. In some embodiments, the nucleic acids of the disclosure are introduced into host cells via transfection.

Electroporation involves application of voltage to create transient pores in the cell membrane, which enables nucleic acids and other macromolecules to enter the cells. In some embodiments, the nucleic acids of the disclosure are introduced into host cells via electroporation.

Lipofection is a liposome-based method that uses a lipid complex to introduce nucleic acids into host cells. Liposomes have the same lipid composition as the cell membrane and contacting a cell membrane with a liposome results in the fusion of the liposome with the cell membrane, releasing the contents encased by the liposome into the cell's cytoplasm. In some embodiments, the host cell is contacted via lipofection with an mRNA or mmRNA comprising the sequence of a gene transfer construct of the disclosure.

Nucleic acids can be introduced to host cells in vivo and methods of introducing nucleic acids to host cells in vivo are known in the art. For instance, a nucleic acid can be introduced to an in vivo host cell via viral and nonviral vectors. A nucleic acid can also be introduced to an in vivo host cell via other methods, e.g., via lipid-based methods such as lipid nanoparticles.

### 6.4. Methods of Expressing Short Peptides in Host Cells and Therapeutic Uses Thereof

Peptides of interest of the disclosure can be introduced into and expressed inside host cells using any of the methods described in Section 6.3.3.

In some embodiments, the host cell is cultured in a medium without a stabilizing agent. The omission of the stabilizing agent enables rapid degradation of DD peptides translated from the gene transfer construct mRNA of the disclosure. Therefore, when the medium lacks the stabilizing agent, the expression product of the gene transfer construct of the disclosure in a successfully transfected host cell is only the peptide of interest.

Alternatively, the host cell can be contacted with the stabilizing agent, for example, by culturing the cell in a medium comprising the stabilizing agent. The cell can be cultured in the medium with the stabilizing agent for a period of time. Without being bound by theory, it is believed that including the stabilizing agent can allow enhanced translation of the polynucleotide sequence encoding the gene transfer constructs, thereby increasing the yield of peptides of interest. Including the stabilizing agent can also be used to assess the efficacy of transfection. For instance, a sequence encoding a reporter or a marker protein can be fused to the sequence of DD, to generate a marker-DD fusion polypeptide. The presence of the stabilizing agent ensures that the marker-DD fusion polypeptide remains intact until transfection is confirmed by the detection of the marker. Detection methods of marker and reporter proteins are well-known in the art. Once the transfection is confirmed, the stabilizing agent can be removed from the medium, leading to the degradation of the marker-DD fusion polypeptide.

In some embodiments, the host cell is cultured in a medium with the stabilizing agent for at least 8 hours (e.g., at least 12 hours, at least 1 day, at least 2 days, or more) and/or up to 5 days (e.g., up to 4 days, up to 3 days, or up to 2 days). Subsequently, the stabilizing agent can be removed, for example by culturing the host cell in a culture medium without the stabilizing agent. Once the stabilizing agent is removed, the DD will be destabilized, leading to degradation of the DD, while leaving the peptides of interest intact.

In other embodiments, the host cell is an in vivo cell, e.g., a cell in a subject that is not treated with a stabilizing agent. Without being bound by theory, it is believed that by not using a stabilizing agent, the peptide of interest can be produced without significant accumulation of DD protein.

In some other embodiments, the subject is treated with the stabilizing agent for a period of time, which without being bound by theory is believed to increase the yield of peptides of interest, or to assess the efficacy of transfection as described supra. Without being bound by theory, it is believed that withdrawal of stabilizing agent can allow the destabilization of the DD, thereby promoting its degradation, while leaving the peptides of interest intact.

Intracellular expression of short peptide inhibitors can be used to treat various disease conditions that involve PPIs. Some nonlimiting examples of diseases that can be targeted with the intracellular expression of the peptides of interest are mitochondrial disorders, proliferative diseases (e.g., cancer), metabolic diseases, infectious diseases, and neurodegenerative diseases.

Under physiological conditions, the balance between mitochondrial fission and fusion in a cell is essential to keep mitochondria of that cell healthy and functional. In contrast, mitochondrial hyperfission leads to mitochondrial fragmentation and damage, decreased mitochondrial membrane potential, increased permeability, and decreased ATP production, and in some cases, apoptosis of otherwise healthy cells. Therefore, methods that reduce mitochondrial hyperfission can prevent changes associated with mitochondrial hyperfission, such as decreased ATP production and apoptosis.

Cell culture studies demonstrated that Drp1-induced excessive mitochondrial fission and fragmentation and plays an active role in apoptosis (Frank et al., 2001, Dev Cell, 1:515-525; Estaquier and Arnoult, 2007, Cell Death Differ, 14:1086-1094), autophagic cell death (Twig et al., 2008, EMBO J 27:433-446; Barsoum et al., 2006, EMBO J 25:3900-3911) and necrosis (Wang et al., 2012, Cell 148:228-243).

Peptide inhibitors of Drp1, such as P110, can prevent mitochondrial hyperfission and fragmentation. Hence, Drp1 inhibitors, such as P110, can be used for treating diseases, which include but are not limited to, mitochondrial disorders, neurodegenerative diseases and age-related disorders that are associated with mitochondrial dysfunction (e.g., ocular or auditory diseases, Huntington's disease, etc.), heart failure, muscle weakness and atrophy, or a proliferative disease.

In some embodiments, the gene transfer constructs of the disclosure, comprising one or more copies of one or more small peptide inhibitors of Drp1, are used to prevent mitochondrial hyperfission. In some embodiments, the gene transfer constructs of the disclosure, comprising one or more copies of one or more small peptide inhibitors of Drp1, are used to treat mitochondrial disorders, as well as other diseases and disorders as mentioned supra.

In some embodiments, the gene transfer construct of the disclosure comprises one or more copies of P110. In some embodiments, the gene transfer construct comprises one copy of P110. In some embodiments, the gene transfer construct comprises two or more copies of P110, such as 2, 3, 4, or 5 copies of P110.

In some embodiments, P110 comprising gene transfer construct are used to treat mitochondrial disorders, neurodegenerative diseases and age-related disorders that are associated with mitochondrial dysfunction, heart failure, muscle weakness and atrophy, or a proliferative disease, or any other disease or disorder, wherein mitochondrial hyperfission is indicated, such as the diseases described in Section 6.4.1.

Small peptide inhibitors can also be used to target cancer cell proliferation. For instance, inhibition of anti-apoptotic Bcl-2 family proteins in cancer cells may induce apoptosis in such cells, including cancer cells that are resistant to conventional chemotherapies. Similarly, peptides that inhibit TOPK, SALL4, Ras, p53, PP2A, STAT3, and YAP may also induce apoptosis in cancer cells.

In some embodiments, the gene transfer constructs of the disclosure comprising one or more copies of one or more small peptide inhibitors of anti-apoptotic Bcl-2 family proteins, are used to induce apoptosis in cancer cells, In some embodiments, the gene transfer constructs of the disclosure comprising one or more copies of one or more small peptide inhibitors of anti-apoptotic Bcl-2 family proteins are used to treat a variety of cancers, such as liver cancer and colorectal cancer.

In some other embodiments, the gene transfer constructs of the disclosure comprising one or more copies of one or more small peptide inhibitors of non-Bcl-2 family proteins associated with unregulated cell division (e.g., TOPK, SALL4, Ras, p53, PP2A, STAT3, YAP) are used to induce apoptosis in cancer cells, In some embodiments, the gene transfer constructs of the disclosure comprising one or more copies of one or more such small peptide inhibitors are used to treat a variety of cancers, such as liver cancer and colorectal cancer.

Accordingly, in another aspect, the disclosure provides a method of treating a subject having an age-related disease, mitochondrial disease or disorder, neurodegenerative disease, retinal disease, diabetes, hearing disorder, genetic disease, heart failure, immunodeficiency, cancer, or infectious disease by in vivo expression of the gene transfer construct or by administering a therapeutically effective number of cells obtained or obtainable by the methods described herein. For example, the subject can have a disease or disorder described in this Section.

### 6.4.1. Methods of Preventing Mitochondrial Hyperfission

In another aspect, the disclosure provides a method for preventing mitochondrial hyperfission in a cell, the method including contacting the cell with a nucleic acid of the disclosure (e.g., mRNA, mmRNA, or expression vector). The contacting can occur in vitro or in vivo.

Short peptides of interest of the disclosure (e.g., P110) can be introduced into and expressed inside host cells using any of the methods described in Section 6.3.3 to prevent mitochondrial hyperfission.

In some embodiments the methods of the disclosure result in the prevention of mitochondrial hyperfission. In some embodiments, the inhibition of mitochondrial hyperfission is associated with reduction of mitochondrial fragmentation, reduced number of damaged mitochondria, increase in mitochondrial membrane potential, restoration of mitochondrial membrane permeability to healthy levels, restoration of ATP production, and reduction of cell death.

Diseases that are associated with mitochondrial hyperfission have been described by Serasinghe and Chipuk (Serasinghe and Chipuk, 2017. Handb Exp Pharmacol. 240:159-188.) and include various atrophies (e.g., muscle atrophies and autosomal dominant optic atrophy), developmental defects and disorders (e.g., abnormal brain development, microencephaly, hypoplasia, and persistent lactic acidemia), neuropathies, neurodegenerative disorders (e.g., Parkinson's disease (PD), Alzheimer's disease (AD), and Huntington's disease (HD)), cardiovascular diseases and cardiomyopathy, metabolic disorders (e.g., type I and type 11 diabetes), and obesity.

The method described herein can be used to generate therapeutic cells to be administered to a subject in order to treat a disease, e.g., one of the aforementioned diseases associated with mitochondrial hyperfission.

In another aspect, the disclosure also provides a method for providing or administering an effective amount of a nucleic acid encoding a gene transfer construct to a subject in order to treat a disease, e.g., one of the aforementioned diseases associated with mitochondrial hyperfission.

### 6.4.2. Methods of Inducing Apoptosis in Cancer Cells

In another aspect, the disclosure provides a method for inducing apoptosis in a cell, the method including contacting the cell with a nucleic acid of the disclosure (e.g., mRNA, mmRNA, or expression vector). The contacting can occur in vitro or in vivo.

Short peptides of interest, such as peptides that inhibit anti-apoptotic Bcl-2 family of proteins, TOPK, SALL4, Ras, p53, PP2A, STAT3, and YAP, can be introduced into and expressed inside host cells using any of the methods described in Section 6.3.3 to induce apoptosis in cells, e.g., in cancer cells.

The method described herein can be used to generate therapeutic cells to be administered to a subject in order to treat various forms of cancer.

In another aspect, the disclosure provides a method for treating a subject having cancer, the method including providing or administering an effective amount of a nucleic acid encoding a gene transfer construct to a subject in order to treat various forms of cancer.

In some embodiments, the cancer is liver cancer or colorectal cancer. In some embodiments, the liver cancer is hepatocellular carcinoma. In some embodiments, the colorectal cancer is a primary tumor or a metastasis. In some embodiments, the cancer is a hematopoetic cancer. In some embodiments, the cancer is an acute myeloid leukemia, a chronic myeloid leukemia, a chronic myelomonocytic leukemia, a myodystrophy (including refractory anemias and refractory cytopenia) or a myeloproliferative neoplasm or disease (including polycythemia vera, essential thrombocytosis and primary myelofibrosis). In other embodiments, the cancer is a blood-based cancer or a hematopoietic cancer.

In some embodiments, the disclosure provides a method, wherein a nucleic acid of the disclosure is used in a combination therapy. Suitable therapeutic agents for use in combination therapy include small molecule chemotherapeutic agents, including protein tyrosine kinase inhibitors, as well as biological anti-cancer agents, such as anti-cancer antibodies.

### 6.5. Pharmaceutical Compositions and Kits

In another aspect, the disclosure provides a pharmaceutical composition comprising a nucleic acid of the disclosure (e.g., as described in Section 6.2 or Section 6.3), or a cell of the disclosure (e.g., a cell described in Section 6.3 or obtained by a method described in 6.4), and a pharmaceutically acceptable excipient. For example, pharmaceutical compositions can be prepared by mixing a nucleic acid or cell with one or more physiologically acceptable carriers, excipients, or stabilizers in the form of, e.g., aqueous solutions or suspensions (see, e.g., Hardman et al., 2001, Goodman and Gilman's The Pharmacological Basis of Therapeutics, McGraw-Hill, New York, N.Y.; Gennaro, 2000, Remington: The Science and Practice of Pharmacy, Lippincott, Williams, and Wilkins, New York, N.Y.; Weiner and Kotkoskie, 2000, Excipient Toxicity and Safety, Marcel Dekker, Inc., New York, N.Y.).

In another aspect, the disclosure provides a pharmaceutical composition comprising a nucleic acid of the disclosure (e.g., an mRNA or mmRNA as described in Sections 6.3.1.1 and 6.3.1.2, respectively) comprising the sequence encoding a gene transfer construct of the disclosure (e.g., as described in Section 6.2), and (a) a lipid compound (e.g., a lipid nanoparticles) and (b) additional components (e.g., phospholipids, structural lipids, polyethylene glycol lipids, quaternary amine compounds, ionizable amino lipids, other lipid composition components and nanoparticle compositions).

A pharmaceutical composition comprising a nucleic acid of the disclosure (e.g., as described in Section 6.3) may be administered to a subject by any suitable route. In some embodiments, compositions of the disclosure are administered by one or more of a variety of routes, including parenteral (e.g., subcutaneous, intracutaneous, intravenous, intraperitoneal, intramuscular, intraarticular, intraarterial, intrasynovial, intrasternal, intrathecal, intralesional, or intracranial injection, as well as any suitable infusion technique), oral, trans- or intra-dermal, interdermal, rectal, intravaginal, topical (e.g., by powders, ointments, creams, gels, lotions, and/or drops), mucosal, nasal, buccal, enteral, vitreal, intratumoral, sublingual, intranasal; by intratracheal instillation, bronchial instillation, and/or inhalation; as an oral spray and/or powder, nasal spray, and/or aerosol, and/or through a portal vein catheter. In some embodiments, a composition may be administered intravenously, intramuscularly, intradermally, intra-arterially, intratumorally, subcutaneously, or by inhalation. However, the present disclosure encompasses the delivery of compositions of the disclosure by any appropriate route taking into consideration likely advances in the sciences of drug delivery. In general, the most appropriate route of administration will depend upon a variety of factors including the nature of the pharmaceutical composition including one or more mRNAs (e.g., its stability in various bodily environments such as the bloodstream and gastrointestinal tract), and the condition of the subject (e.g., whether the subject is able to tolerate particular routes of administration).

In another aspect, the disclosure provides kits comprising a nucleic acid of the disclosure (e.g., as described in Section 6.3) and a stabilizing agent. For example, the kit can include trimethoprim (TMP) or methotrexate (MTX) when the DD sequence of the gene transfer construct is a DHFR DD sequence; Shield-1, rapamycin, or FK506 when the DD sequence of the gene transfer construct is a FKBP DD sequence; sildenafil, vardenafil, tadalafil, avanafil, lodenafil, mirodenafil, udenafil, benzamidenafil, dasantafil, or beminafil when the DD sequence of the gene transfer construct is a PDE5 DD sequence; Celecoxib (Celebrex), Valdecoxib, Rofecoxib (Vioxx), Acetazolamide, Methazolamide, Dorzolamide, Brinzolamide, Diclofenamide, Ethoxzolamide, Zonisamide, Dansylamide, and Dichlorphenamide when the DD sequence of the gene transfer construct is a CA2 DD sequence; Pioglitazone and Posiglitazone when the DD sequence of the gene transfer construct is a PPAR gamma DD; or Imatinib and Melatonin when the DD sequence of the gene transfer construct is an NQO2 DD.

### 7. Specific Embodiments

The present disclosure is exemplified by the specific embodiments below.

1. A mRNA molecule encoding:
(a) a destabilizing domain (DD);
(b) a translational separator; and
(c) a peptide of interest.

2. The mRNA of embodiment 1, wherein the DD is a protein, a peptide, or a peptide fragment that is rapidly degraded upon translation of the mRNA in the absence of a stabilizing molecule.

3. The mRNA of embodiment 1 or embodiment 2, wherein the DD-encoding sequence enhances ribosomal targeting of the mRNA as compared to a mRNA encoding the translational separator and the peptide of interest but not the destabilizing domain.

4. The mRNA of any one of embodiments 1 to 3, wherein the DD is a dihydrofolate reductase (DHFR), a FK506-binding protein (FKBP), a carbonic anhydrase 2 (CA2)-derived DD, a phosphodiesterase 5 (PDE5)-derived DD, a peroxisome proliferator-activated receptor gamma (PPAR gamma)-derived DD, a NRH:quinone oxidoreductase 2 (NQO2)-derived DD, a human estrogen receptor ligand binding domain (ERLBD)-derived DD, or an UnaG-derived DD.

5. The mRNA of any one of embodiments 1 to 4, wherein the nucleotide sequence encoding DD is codon-optimized for expression in human cells.

6. The mRNA of any one of embodiments 1 to 5, the DD is a DHFR (e.g., an E. coli DHFR (ecDHFR)).

7. The mRNA of embodiment 6, wherein the nucleotide sequence encoding the DD encodes a DD comprising the amino acid sequence of SEQ ID NO:1.

8. The mRNA of embodiment 6, wherein the nucleotide sequence encoding the DD encodes a DD comprising the amino acid sequence of SEQ ID NO:2.

9. The mRNA of embodiment 6, wherein the nucleic acid sequence encoding ecDHFR comprises

(SEQ ID NO: 3)
Atcagtctgattgcggcgttagcggtagatcacgttatcggcatggaaaccgtcatgccgtggaacctgcctgccgatctcgcctg
gtttaaacgcaacaccttaaataaacccgtgattatgggccgccatacctgggaatcaatcggtcgtccgttgccaggacgcaaa
aatattatcctcagcagtcaaccgagtacggacgatcgcgtaacgtgggtgaagtcggtggatgaagccatcgcggcgtgtggt
gacgtaccagaaatcatggttattggcggcggtcgcgtttatgaacagttcttgccaaaagcgcaaaaactgtatctgacgcatat
cgacgcagaagtggaaggcgacacccatttcccggattacgagccggatgactgggaatcggtattcagcgaattccacgatg
ctgatgcgcagaactctcacagctattgctttgagattctggagcggcgataa

10. The mRNA of any one of embodiments 1 to 5, wherein the DD is a FK506-binding protein (FKBP).

11. The mRNA of embodiment 10, wherein the nucleotide sequence encoding the DD encodes a DD comprising the amino acid sequence of SEQ ID NO:4.

12. The mRNA of any one of embodiments 1 to 5, wherein the DD is a carbonic anhydrase 2 (CA2)-derived DD.

13. The mRNA of embodiment 12, wherein the nucleotide sequence encoding the DD encodes a DD comprising the amino acid sequence of SEQ ID NO:9.

14. The mRNA of any one of embodiments 1 to 5, wherein the DD is a phosphodiesterase 5 (PDE5)-derived DD.

15. The mRNA of embodiment 14, wherein the nucleotide sequence encoding the DD encodes a DD comprising the amino acid sequence of SEQ ID NO:5.

16. The mRNA of embodiment 14, wherein the nucleotide sequence encoding the DD encodes a DD comprising the amino acid sequence of SEQ ID NO:6.

17. The mRNA of embodiment 14, wherein the nucleotide sequence encoding the DD encodes a DD comprising the amino acid sequence of SEQ ID NO:7.

18. The mRNA of embodiment 14, wherein the nucleotide sequence encoding the DD encodes a DD comprising the amino acid sequence of SEQ ID NO:8.

19. The mRNA of any one of embodiments 1 to 5, wherein the DD is a peroxisome proliferator-activated receptor gamma (PPAR gamma)-derived DD.

20. The mRNA of embodiment 19, wherein the nucleotide sequence encoding the DD encodes a DD comprising the amino acid sequence of SEQ ID NO:10.

21. The mRNA of embodiment 19, wherein the nucleotide sequence encoding the DD encodes a DD comprising the amino acid sequence of SEQ ID NO:11.

22. The mRNA of any one of embodiments 1 to 5, wherein the DD is a NRH:quinone oxidoreductase 2 (NQO2)-derived DD.

23. The mRNA of embodiment 22, wherein the nucleotide sequence encoding the DD encodes a DD comprising the amino acid sequence of SEQ ID NO:12.

24. The mRNA of any one of embodiments 1 to 5, wherein the DD is a human estrogen receptor ligand binding domain (ERLBD)-derived DD.

25. The mRNA of embodiment 24, wherein the nucleotide sequence encoding the DD encodes a DD comprising the amino acid sequence of SEQ ID NO:13.

26. The mRNA of any one of embodiments 1 to 5, wherein the DD is a an UnaG-derived DD.

27. The mRNA of embodiment 26, wherein the nucleotide sequence encoding the DD encodes a DD comprising the amino acid sequence of SEQ ID NO:14.

28. The mRNA of any one of embodiments 1 to 27, wherein the nucleotide sequence encoding the translational separator is located 3′ to the nucleotide sequence encoding the DD and 5′ to the nucleotide sequence encoding the peptide of interest.

29. The mRNA of any one of embodiments 1 to 28, wherein the translational separator is a self-cleaving peptide.

30. The mRNA of embodiment 29, wherein the self-cleaving peptide is a P2A, E2A, F2A, or T2A self-cleaving peptide.

31. The mRNA of embodiment 30, wherein the self-cleaving peptide is a P2A self-cleaving peptide.

32. The mRNA of embodiment 31, wherein the amino acid sequence of the P2A self-cleaving peptide comprises DVEXNPGP (SEQ ID NO:15).

33. The mRNA of embodiment 31, wherein the amino acid sequence of the P2A self-cleaving peptide comprises ATNFSLLKQAGDVEENPGP (SEQ ID NO:16).

34. The mRNA of embodiment 31, wherein the amino acid sequence of the P2A self-cleaving peptide comprises GSGATNFSLLKQAGDVEENPGP (SEQ ID NO:17).

35. The mRNA of embodiment 30, wherein the self-cleaving peptide is an E2A self-cleaving peptide.

36. The mRNA of embodiment 35, wherein the amino acid sequence of the E2A self-cleaving peptide comprises QCTNYALLKLAGDVESNPGP (SEQ ID NO:18).

37. The mRNA of embodiment 35, wherein the amino acid sequence of the E2A self-cleaving peptide comprises GSGQCTNYALLKLAGDVESNPGP (SEQ ID NO:19).

38. The mRNA of embodiment 30, wherein the self-cleaving peptide is an F2A self-cleaving peptide.

39. The mRNA of embodiment 38, wherein the amino acid sequence of the F2A self-cleaving peptide comprises VKQTLNFDLLKLAGDVESNPGP (SEQ ID NO:20).

40. The mRNA of embodiment 38, wherein the amino acid sequence of the F2A self-cleaving peptide comprises GSGVKQTLNFDLLKLAGDVESNPGP (SEQ ID NO:21).

41. The mRNA of embodiment 30, wherein the self-cleaving peptide is a T2A self-cleaving peptide.

42. The mRNA of embodiment 41, wherein the amino acid sequence of the T2A self-cleaving peptide is EGRGSLLTCGDVEENPGP (SEQ ID NO:22).

43. The mRNA of embodiment 41, wherein the amino acid sequence of the T2A self-cleaving peptide is GSGEGRGSLLTCGDVEENPGP (SEQ ID NO:23).

44. The mRNA of any one of embodiments 1 to 43, wherein the nucleotide sequence encoding the self-cleaving peptide is codon-optimized for expression in human cells.

45. The mRNA of any one of embodiments 1 to 44, wherein the nucleotide sequence encoding the peptide of interest is codon-optimized for expression in human cells.

46. The mRNA of any one of embodiments 1 to 45, wherein the peptide of interest is a peptide that cannot be effectively delivered into target cells via conventional drug delivery methods.

47. The mRNA of any one of embodiments 1 to 46, wherein the peptide of interest is 2 to 150 amino acids in length.

48. The mRNA of embodiment 47, wherein the peptide of interest is at least 3 amino acids in length.

49. The mRNA of embodiment 47, wherein the peptide of interest is at least 4 amino acids in length.

50. The mRNA of embodiment 47, wherein the peptide of interest is at least 5 amino acids in length.

51. The mRNA of embodiment 47, wherein the peptide of interest is at least 7 amino acids in length.

52. The mRNA of embodiment 47, wherein the peptide of interest is at least 10 amino acids in length.

53. The mRNA of embodiment 47, wherein the peptide of interest is at least 20 amino acids in length.

54. The mRNA of any one of embodiments 47 to 53, wherein the peptide of interest is no more than 130 amino acids in length.

55. The mRNA of any one of embodiments 47 to 53, wherein the peptide of interest is no more than 100 amino acids in length.

56. The mRNA of any one of embodiments 47 to 53, wherein the peptide of interest is no more than 80 amino acids in length.

57. The mRNA of any one of embodiments 47 to 53, wherein the peptide of interest is no more than 60 amino acids in length.

58. The mRNA of any one of embodiments 47 to 53, wherein the peptide of interest is no more than 40 amino acids in length.

59. The mRNA of any one of embodiments 47 to 53, wherein the peptide of interest is no more than 20 amino acids in length.

60. The mRNA of any one of embodiments 47 to 52, wherein the peptide of interest is no more than 10 amino acids in length.

61. The mRNA of any one of embodiments 1 to 46, wherein the peptide of interest is 3 to 100 amino acids in length.

62. The mRNA of any one of embodiments 1 to 46, wherein the peptide of interest is 4 to 50 amino acids in length.

63. The mRNA of any one of embodiments 1 to 46, wherein the peptide of interest is 5 to 20 amino acids in length.

64. The mRNA of any one of embodiments 1 to 46, wherein the peptide of interest is 6 to 10 amino acids in length.

65. The mRNA of any one of embodiments 1 to 64, wherein the peptide of interest is a therapeutic peptide.

66. The mRNA of any one of embodiments 1 to 65, wherein the peptide of interest is capable of inhibiting protein-protein interactions.

67. The mRNA of any one of embodiments 1 to 66, wherein the peptide of interest is an inhibitor of dynamin-1-related protein (Drp1), T-lymphokine-activated killer cell-originated protein kinase (TOPK), Sal-like protein 4 (SALL4), Ras, p53, protein phosphatase 2A (PP2A), signal transducer and activator of transcription 3 (STAT3), Yes-associated protein (YAP), a Bcl-2 family protein, NOTCH, an estrogen receptor, microtubule-associated protein light chain 3 (LC3), or MDM2/MDMX.

68. The mRNA of any one of embodiments 1 to 67, wherein the peptide of interest is a peptide listed in Table 2.

69. The mRNA of any one of embodiments 1 to 68, wherein the peptide of interest is a Drp1 inhibitor.

70. The mRNA of any one of embodiments 1 to 69, wherein the peptide of interest is P110.

71. The mRNA of embodiment 70, wherein the nucleotide sequence encoding P110 is codon-optimized for expression in human cells.

72. The mRNA of embodiment 70 or embodiment 71, wherein the nucleotide sequence encoding P110 comprises GATCTGCTGCCACGCGGGACG (SEQ ID NO:25).

73. The mRNA of any one of embodiments 1 to 68, wherein the peptide of interest is an inhibitor of T-lymphokine-activated killer cell-originated protein kinase (TOPK).

74. The mRNA of any one of embodiments 1 to 68, wherein the peptide of interest is an inhibitor of Sal-like protein 4 (SALL4).

75. The mRNA of any one of embodiments 1 to 68, wherein the peptide of interest is an inhibitor of Ras.

76. The mRNA of any one of embodiments 1 to 68, wherein the peptide of interest is an inhibitor of p53.

77. The mRNA of any one of embodiments 1 to 68, wherein the peptide of interest is an inhibitor of protein phosphatase 2A (PP2A).

78. The mRNA of any one of embodiments 1 to 68, wherein the peptide of interest is an inhibitor of signal transducer and activator of transcription 3 (STAT3).

79. The mRNA of any one of embodiments 1 to 68, wherein the peptide of interest is an inhibitor of Yes-associated protein (YAP).

80. The mRNA of any one of embodiments 1 to 68, wherein the peptide of interest is an inhibitor of a Bcl-2 family protein.

81. The mRNA of any one of embodiments 1 to 68, wherein the peptide of interest is an inhibitor of NOTCH.

82. The mRNA of any one of embodiments 1 to 68, wherein the peptide of interest is an inhibitor of an estrogen receptor.

83. The mRNA of any one of embodiments 1 to 68, wherein the peptide of interest is an inhibitor of microtubule-associated protein light chain 3 (LC3).

84. The mRNA of any one of embodiments 1 to 68, wherein the peptide of interest is an inhibitor of MDM2/MDMX.

85. The mRNA of any one of embodiments 1 to 84, wherein the mRNA molecule encodes a single copy of the peptide of interest.

86. The mRNA of any one of embodiments 1 to 84, wherein the mRNA molecule encodes two or more copies of the peptide of interest.

87. The mRNA of embodiment 86, wherein the mRNA molecule encodes two copies of the peptide of interest.

88. The mRNA of embodiment 86, wherein the mRNA molecule encodes three copies of the peptide of interest.

89. The mRNA of embodiment 86, wherein the mRNA molecule encodes four copies of the peptide of interest.

90. The mRNA of embodiment 86, wherein the mRNA molecule encodes five copies of the peptide of interest.

91. The mRNA of any one of embodiments 86 to 90, wherein the nucleotide sequence encoding each copy of the peptide of interest is identical.

92. The mRNA of any one of embodiments 86 to 90, wherein the nucleotide sequences encoding the two or more copies of the peptide of interest are not identical.

93. The mRNA of any one of embodiments 1 to 92, wherein the mRNA further encodes a second peptide of interest that is different from the first peptide of interest.

94. The mRNA of embodiment 93, wherein the mRNA further encodes a third peptide of interest that is different from the first and second peptides of interest.

95. The mRNA of any one of embodiments 1 to 94, wherein the mRNA encodes four or more different peptides of interest.

96. The mRNA of any one of embodiments 1 to 95, wherein the mRNA encodes four different peptides of interest.

97. The mRNA of any one of embodiments 1 to 95, wherein the mRNA encodes five different peptides of interest.

98. The mRNA of any one of embodiments 86 to 97, wherein each sequence encoding a peptide of interest is separated by a translational separator, optionally wherein each translational separator is independently selected from translational separators described in embodiments 30 to 44.

99. The mRNA of any one of embodiments 1 to 98, which further encodes a marker protein.

100. A mRNA molecule encoding:
(a) a peptide of interest;
(b) a means for destabilizing an amino acid chain; and
(c) a means for separating the peptide of interest and the means for destabilizing an amino acid chain.

101. The mRNA molecule of embodiment 100, wherein the peptide of interest is a peptide of interest described in any one of embodiments 1 to 99, optionally wherein the means for destabilizing an amino acid chain is a destabilizing domain described in any one of embodiments 1 to 99, and optionally wherein the means for separating the peptide of interest and the means for destabilizing an amino acid chain is a translational separator described in any one of embodiments 1 to 99.

102. A DNA encoding the mRNA of any one of embodiments 1 to 101.

103. The DNA of embodiment 102, which further comprises one or more regulatory elements, optionally wherein one or more regulatory elements comprise a TATA box, a CAAT box, a GC box, a promoter or a combination thereof.

104. The DNA of embodiment 102, which further comprises a promoter operably linked to the sequence encoding the mRNA.

105. The DNA of embodiment 104, wherein the promoter is a CMV early enhancer/chicken beta actin (CAG) promoter.

106. The DNA of any one of embodiments 102 to 105, which further comprises a polyadenylation signal sequence 3′ to the sequence encoding the peptide of interest.

107. The DNA of any one of embodiments 102 to 106, which is an expression plasmid.

108. The DNA of embodiment 107, wherein the expression plasmid is pCAGGS plasmid.

109. The DNA of any one of embodiments 102 to 106, which is a viral genome.

110. The DNA of embodiment 109, wherein the viral genome is an AAV genome.

111. A particle comprising the mRNA of any one of embodiments 1 to 101 or the DNA of any one of embodiments 102 to 110, which is optionally a viral particle or a lipid particle.

112. A pharmaceutical composition comprising the mRNA of any one of embodiments 1 to 101, the DNA of any one of embodiments 102 to 110 or the particle of embodiment 111, and one or more excipients.

113. A host cell comprising the mRNA of any one of embodiments 1 to 101, the DNA of any one of embodiments 102 to 110 or the particle of embodiment 111.

114. A method of introducing a mRNA into a cell, comprising contacting the cell with the mRNA of any one of embodiments 1 to 101, the DNA of any one of embodiments 102 to 110, the particle of embodiment 111, or the pharmaceutical composition of embodiment 112.

115. The method of embodiment 114, which comprises contacting the cell with the mRNA via lipofection.

116. A method of introducing a DNA into a cell via transfection of the cell with the expression plasmid of embodiment 107 or embodiment 108.

117. A method of producing an mRNA comprising transcribing the mRNA from the DNA of any of the embodiments 102 to 110.

118. A method of expressing a peptide of interest in a cell, comprising contacting the cell with the mRNA of any one of embodiments 1 to 101, the DNA of any one of embodiments 102 to 110, the particle of embodiment 111, or the pharmaceutical composition of embodiment 112.

119. A method of inhibiting Drp1/Fis1 binding in a cell, comprising contacting the cell with an mRNA of any one of embodiments 1 to 101 that encodes a Drp1 inhibitor such as P110, the DNA of any one of embodiments 102 to 110 that encodes a Drp1 inhibitor such as P110, or the pharmaceutical composition of embodiment 112 that comprises mRNA or DNA encoding a Drp1 inhibitor such as P110.

120. A method of inhibiting mitochondrial hyperfission in a cell, comprising contacting the cell with an mRNA of any one of embodiments 1 to 101 that encodes a Drp1 inhibitor such as P110, the DNA of any one of embodiments 102 to 110 that encodes a Drp1 inhibitor such as P110, or the pharmaceutical composition of embodiment 112 that comprises mRNA or DNA encoding a Drp1 inhibitor such as P110.

121. A method of inhibiting apoptosis of the mitochondrial pathway in a cell, comprising contacting the cell with an mRNA of any one of embodiments 1 to 101 that encodes a Drp1 inhibitor such as P110, the DNA of any one of embodiments 102 to 110 that encodes a Drp1 inhibitor such as P110, or the pharmaceutical composition of embodiment 112 that comprises mRNA or DNA encoding a Drp1 inhibitor such as P110.

122. A method of inducing apoptosis in a cancer cell, comprising contacting the cell with an mRNA of any one of embodiments 1 to 101 that encodes a peptide that inhibits an Bcl-2 family protein, TOPK, SALL4, Ras, p53, PP2A, STAT3, or YAP, the DNA of any one of embodiments 102 to 110 that encodes a peptide that inhibits an Bcl-2 family protein, TOPK, SALL4, Ras, p53, PP2A, STAT3, or YAP, or the pharmaceutical composition of embodiment 112 that comprises mRNA or DNA encoding a peptide that inhibits an Bcl-2 family protein, TOPK, SALL4, Ras, p53, PP2A, STAT3, or YAP.

123. The method of any one of embodiments 114 to 122, wherein the contacting is ex vivo.

124. The method of any one of embodiments 114 to 122, wherein the contacting is in vivo.

125. The method of any one of embodiments 114 to 124, which comprises contacting the cell with a DD-stabilizing molecule for an initial period of time.

126. The method of embodiment 125, further comprising withdrawing the DD-stabilizing molecule.

127. The method of embodiment 125 or embodiment 126, wherein the DD is a DHFR and the stabilizing molecule is trimethoprim (TMP).

128. The method of any one of embodiments 114 to 124, wherein the cell is not contacted with a DD-stabilizing molecule.

129. A method of treating a neurodegenerative disease, cardiovascular disease, or diabetes, comprising administering to the subject in need thereof a therapeutically effective amount of the mRNA of any one of embodiments 1 to 101 that encodes P110, the DNA of any one of embodiments 102 to 110 that encodes P110, or the pharmaceutical composition of embodiment 112 that comprises mRNA or DNA encoding P110.

130. A method of treating a neurodegenerative disease, cardiovascular disease, or diabetes, comprising administering to the subject in need thereof a therapeutically effective amount of the mRNA of any one of embodiments 1 to 101 that encodes a Drp1 inhibitor such as P110, the DNA of any one of embodiments 102 to 110 that encodes a Drp1 inhibitor such as P110, or the pharmaceutical composition of embodiment 112 that comprises mRNA or DNA encoding a Drp1 inhibitor such as P110.

131. The method of embodiment 129 or embodiment 130, wherein the subject has a neurodegenerative disease.

132. The method of embodiment 129 or embodiment 130, wherein the subject has a cardiovascular disease.

133. The method of embodiment 129 or embodiment 130, wherein the subject has diabetes.

### 8. EXAMPLES

### 8.1. Example 1: Peptide Expression Unit Design

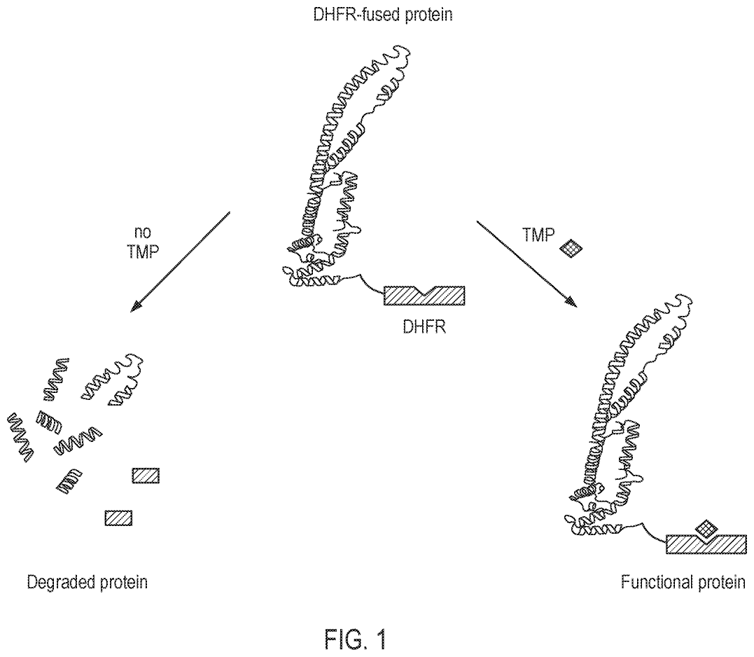

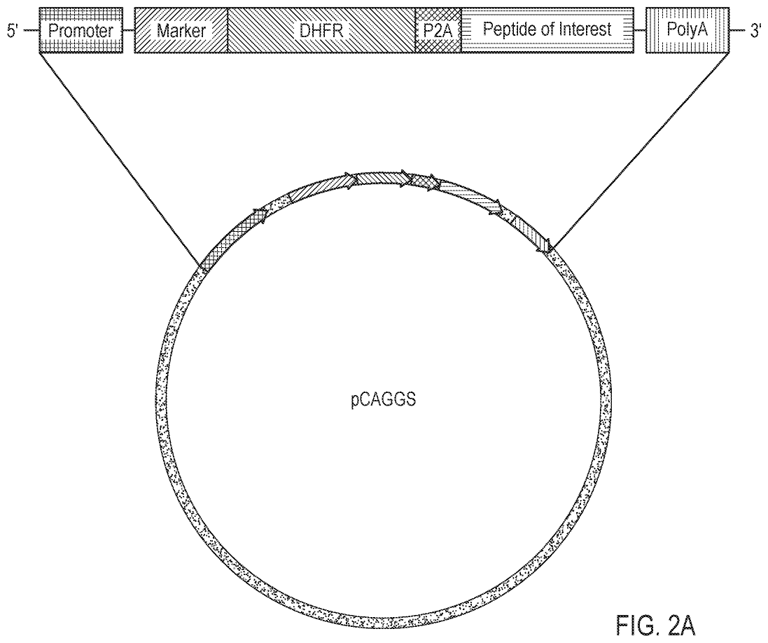

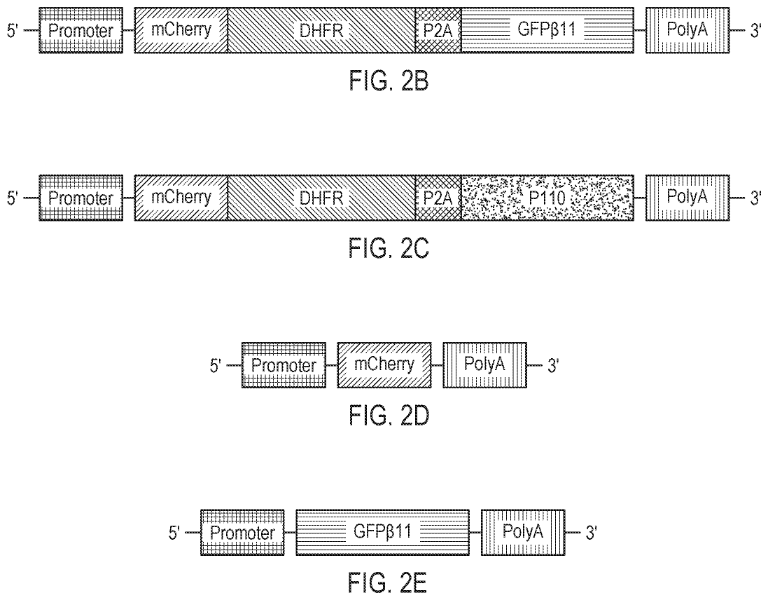

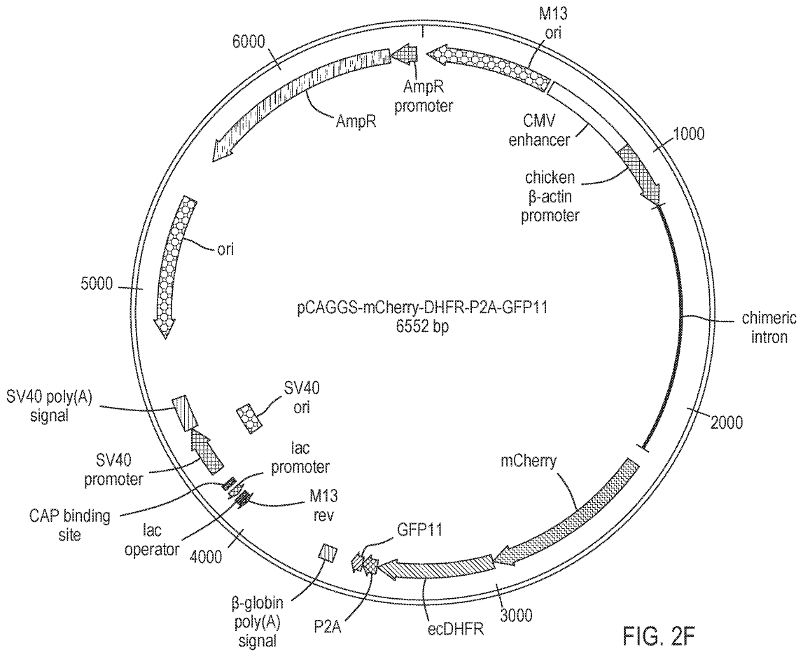

Taking advantage of the rapid degradation of dihydrofolate reductase (DHFR) with a destabilizing domain (DD) in the absence of a stabilizing drug (e.g., trimethoprim (TMP)) (FIG. 1), a gene transfer system was conceived using DHFR as a ribosome binding fragment. A translational separator (e.g., P2A) sequence, was incorporated between the sequences encoding DHFR and the peptide of interest to generate individual peptide products instead of fusion polypeptides. Given that DHFR is rapidly targeted for degradation in the absence of TMP, this design would result in the expression of only the peptide of interest in target cells. Therefore, peptide expression units were designed to comprise, from 5′ to 3′, an optional marker protein (e.g., an mCherry reporter) coding sequence, a sequence encoding dihydrofolate reductase (DHFR), a translational separator (e.g., a P2A peptide) sequence, and the sequence encoding the peptide of interest (FIG. 2). The peptide expression unit was then incorporated between a promoter and a polyA sequence of a suitable expression vector, such as pCAGGS (FIG. 2A).

### 8.2. Example 2: Gene Transfer Using the Peptide Expression Unit

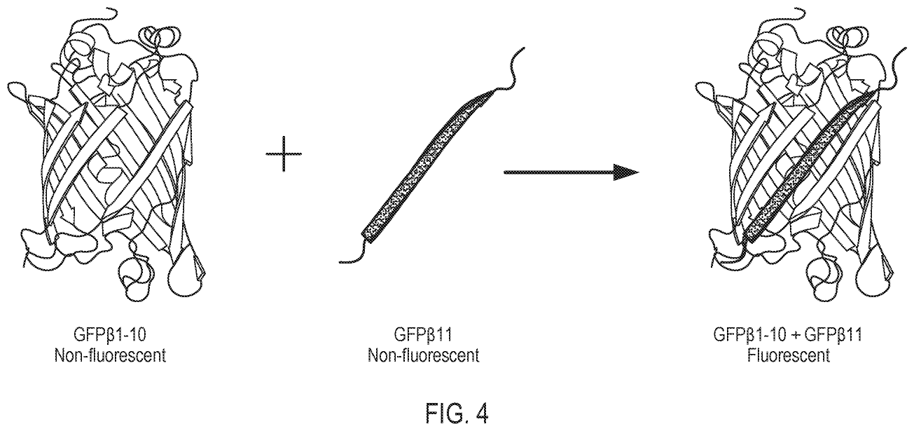

The split GFP system relies on the association of two non-fluorescent polypeptides, GFP p strands 1-10 (GFPP1-10) and GFP p strand 11 (GFPP11), to form a fluorescent molecule (FIG. 4). Hence, the split GFP system was used to evaluate gene transfer using the peptide expression unit described in Example 1, wherein GFPP1-10-expressing cells were transfected with an expression plasmid generated with a peptide expression unit comprising GFPP11.

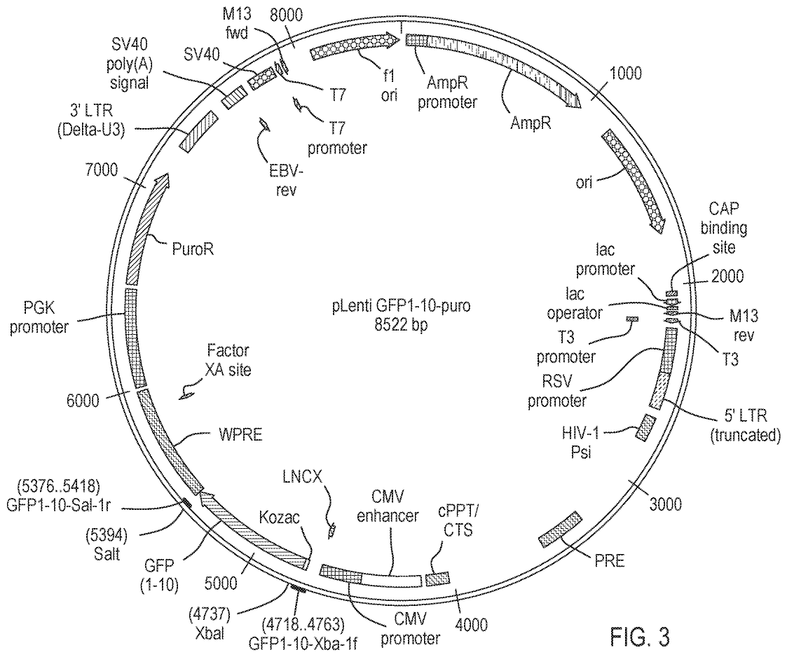

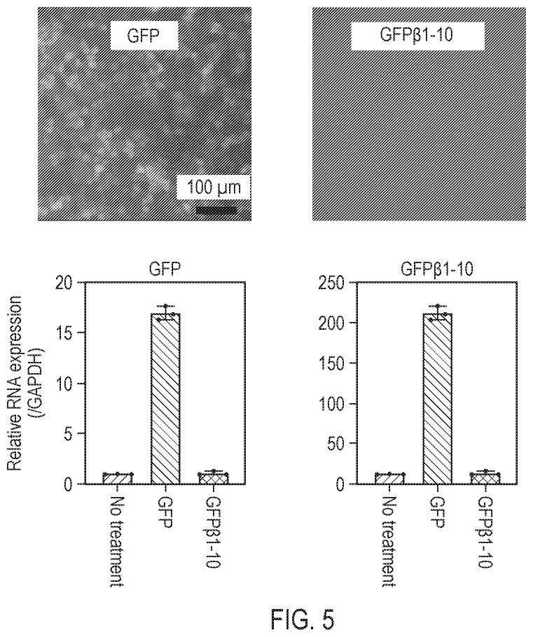

Briefly, a pCAGGS expression plasmid was generated, by incorporating the coding sequences of mCherry, E. coli DHFR (ecDHFR), P2A, and GFPP11, between the chicken p actin promoter and the β-globin poly(A) signal sequence (FIGS. 2B and 2F). Since the pCAGGS expression plasmids used in this example have limited gene transfer efficiency, mCherry fused to ecDHFR was used to evaluate only the cells with successful gene transfer in the presence of TMP. Next, A549 cells were transfected with a pLenti-GFPP1-10-puro plasmid (FIG. 3) to generate cells that express GFPP1-10. After confirming that A549 cells expressed GFPP1-10 but not full-length GFP (FIG. 5), they were transfected with either an expression plasmid comprising the mCherry-DHFR-P2A-GFPβ11 peptide expression unit (FIG. 2B), or a control plasmid, such as an mCherry-only plasmid (FIG. 2D) or a GFPP11-only plasmid (FIG. 2E) or were left untreated.

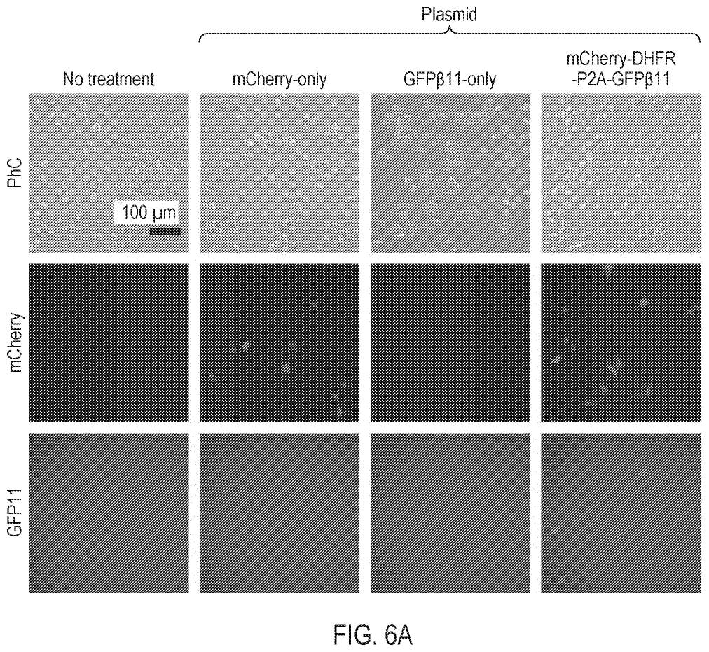

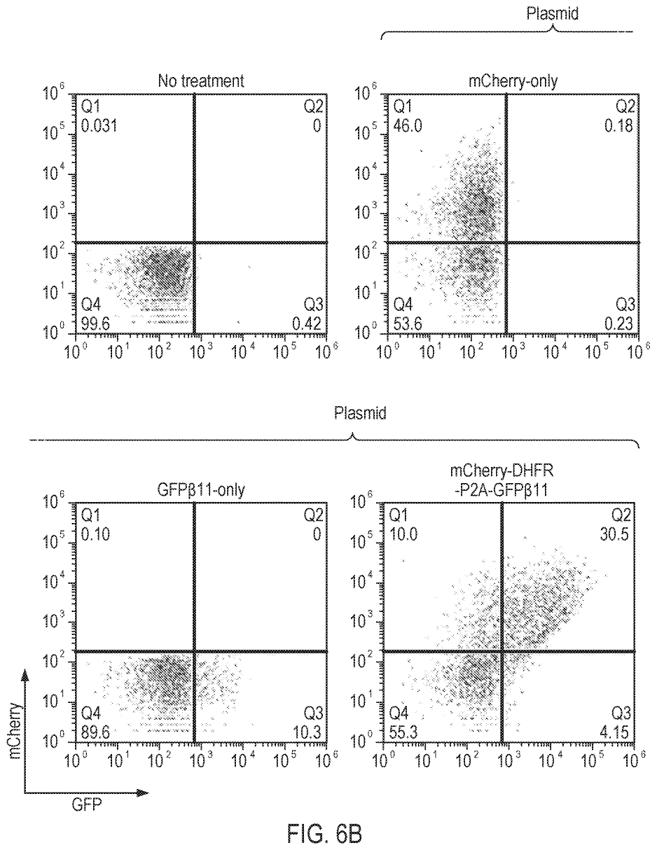

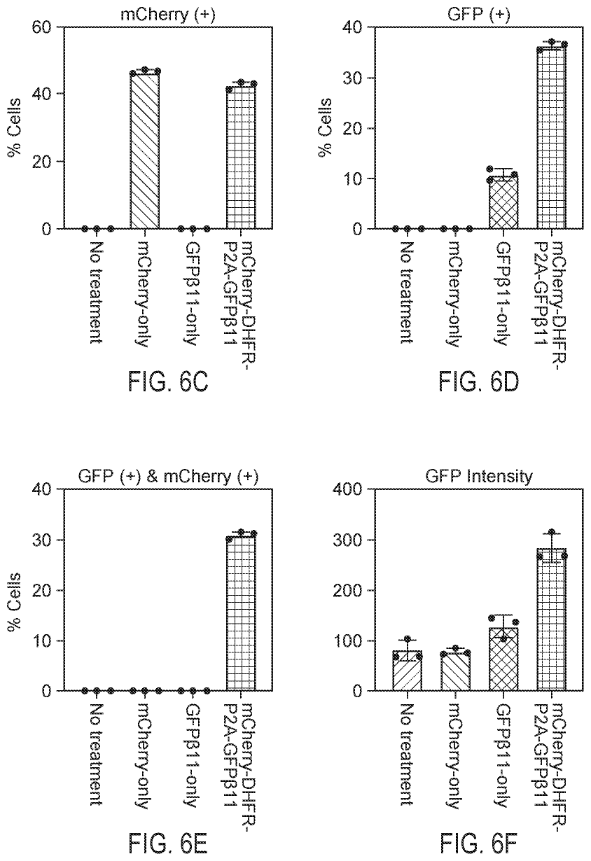

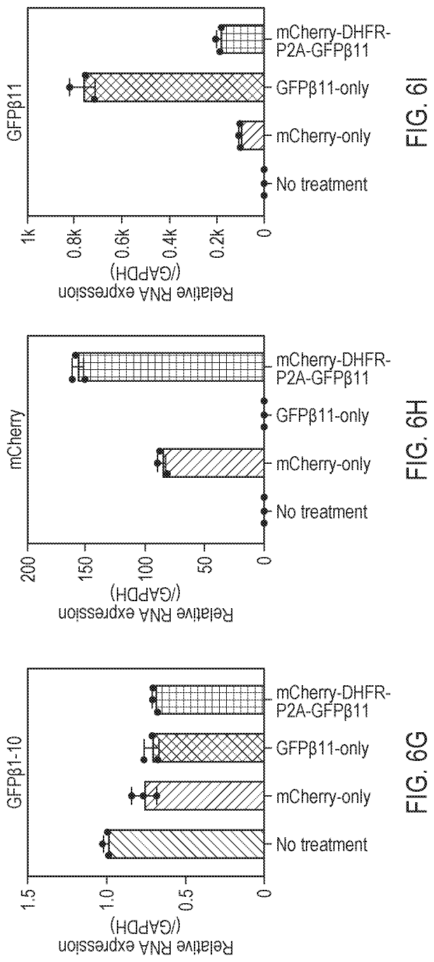

No GFP fluorescence was observed in A549 cells transfected with an mCherry-only control plasmid or a GFPP11-only control plasmid (FIG. 6A). In contrast, most of the mCherry-positive cells transfected with the expression plasmid containing the peptide expression unit were GFP-positive (FIG. 6A). FACS analysis revealed a very small fraction of transgenic cells transfected with GFPP11-only plasmid expressed GFP at a low intensity (FIG. 6B); however, most mCherry-expressing cells transfected with a plasmid comprising the peptide expression unit were GFP-positive (FIGS. 6B-6F).

Next, mRNA expression levels of GFPP1-10, GFPP11, and mCherry were assessed in GFPP11-10-positive A549 cells that were transfected with one of the three expression vectors (m-Cherry-only, GFPP11-only, or mCherry-DHFR-P2A-GFPβ11 peptide expression unit) or were left untreated (FIGS. 6G-6I). The expression of GFPP11 was significantly higher in cells transfected with the GFPP11-only expression vector than in cells transfected with the expression vector comprising the peptide expression unit (FIG. 6I), indicating shorter expression units are associated with higher transcription efficiencies. Nevertheless, not only did almost all cells transfected with the expression vector comprising the mCherry-DHFR-P2A-GFPβ11 peptide expression unit express GFP, but they also displayed the highest GFP intensity (FIG. 6F). Taken together, these results suggest that mRNAs from short expression units are translated inefficiently, regardless of their abundance, whereas mRNAs from recombinant peptide expression units are translated with much more efficiency.

### 8.3. Example 3: DHFR Degrades Rapidly After Translation

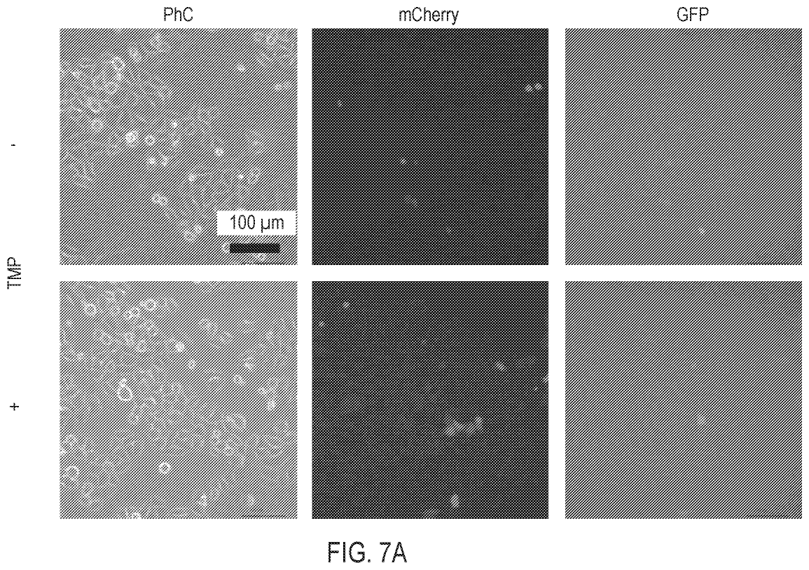

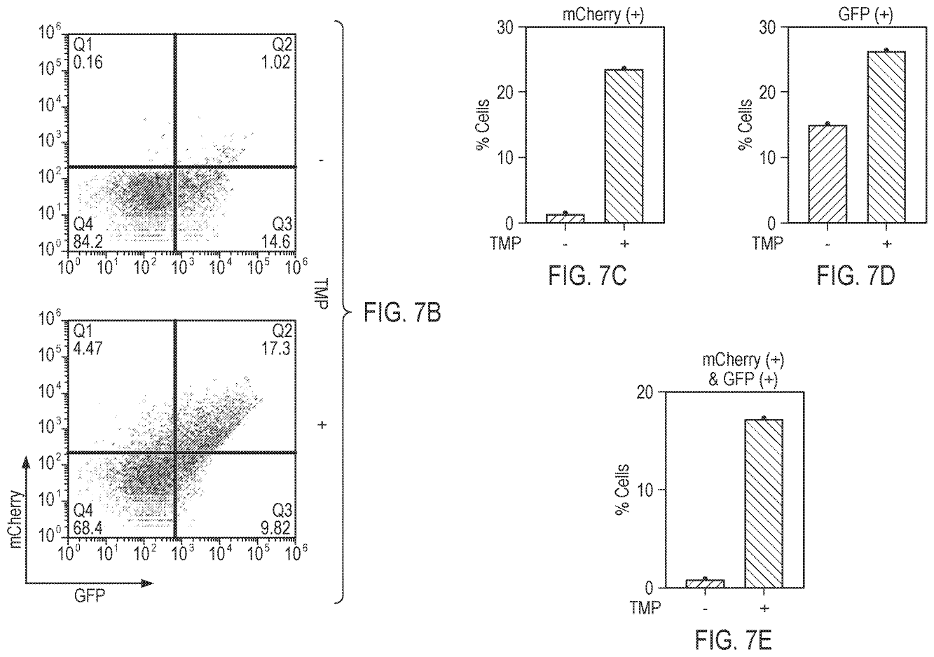

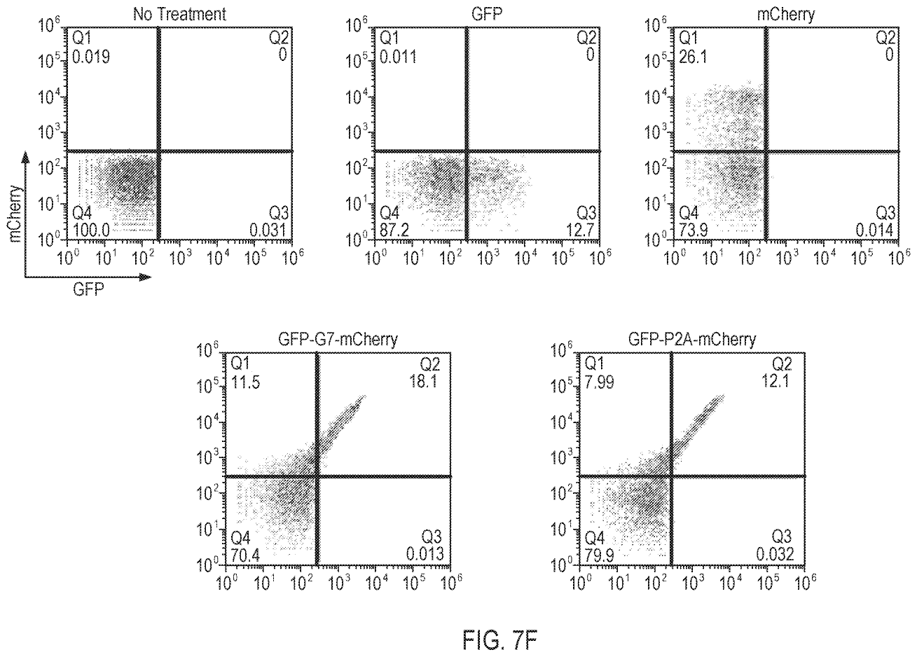

The mCherry-DHFR-P2A-GFPβ11 peptide expression unit was introduced into GFPP1-10 expressing A549 cells and mCherry and GFP expression levels were examined in the presence and absence of TMP. When TMP was present, most mCherry-positive cells were also positive for GFP, whereas GFP expression was very low in these cells in the absence of TMP (FIG. 7A).

FACS analysis revealed that mCherry expression increased sharply following the addition of TMP (FIG. 7B), suggesting the stabilization of the mCherry-DHFR fusion protein by TMP. Indeed, the percentage of cells expressing mCherry upon TMP treatment was similar in cells transfected with an m-Cherry only plasmid and cells transfected with a plasmid comprising the mCherry-DHFR-P2A-GFPβ11 peptide expression unit (FIGS. 7C and 7E). Yet, the percentage of cells with GFP signal was somewhat lower in the absence of TMP relative to its expression in the presence of TMP (FIG. 7D), suggesting TMP exposure enabled more cells to express GFPP11. The expression levels of GFP and mCherry were not greatly affected by the translational separator, as cells transfected with plasmids comprising GFP and mCherry connected via P2A (GFP-P2A-mCherry) or via a control separator sequence (GFP-T7-mCherry) displayed similar diagonal linear signal profiles (FIG. 7F).

Taken together, administering TMP only at the beginning of transduction can be used to enhance the expression of the peptide of interest and subsequent withdrawal of TMP would allow degradation of DHFR, leaving only the peptide of interest to be expressed in target cells.

### 8.4. Example 4: Gene Transfer of P110 Peptide Using Peptide Expression Unit mRNA Suppresses LPS-Induced Hyperfission

Clinical applicability of the peptide expression unit as an mRNA medicine was evaluated using P110, which is a peptide that was developed to suppress mitochondrial hyperfission formation, a process linked to environmental stress-induced apoptosis.

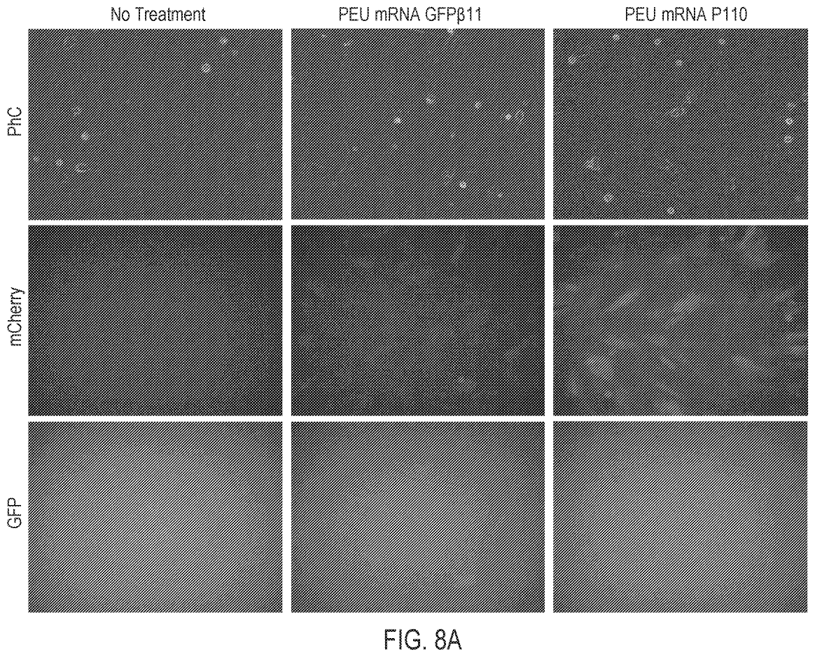

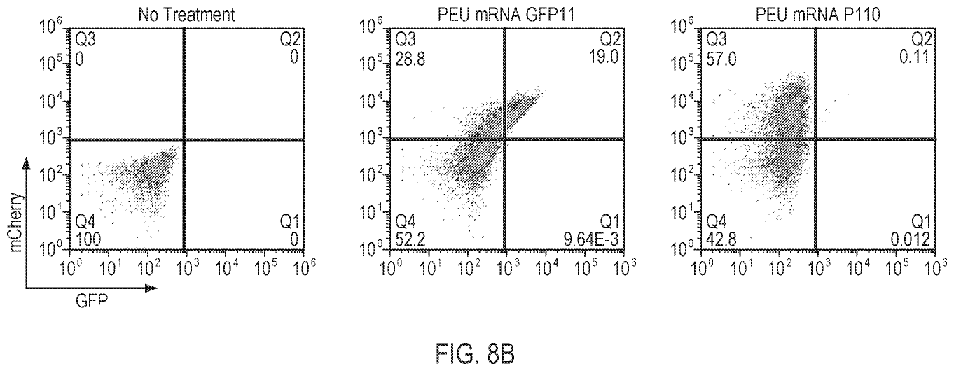

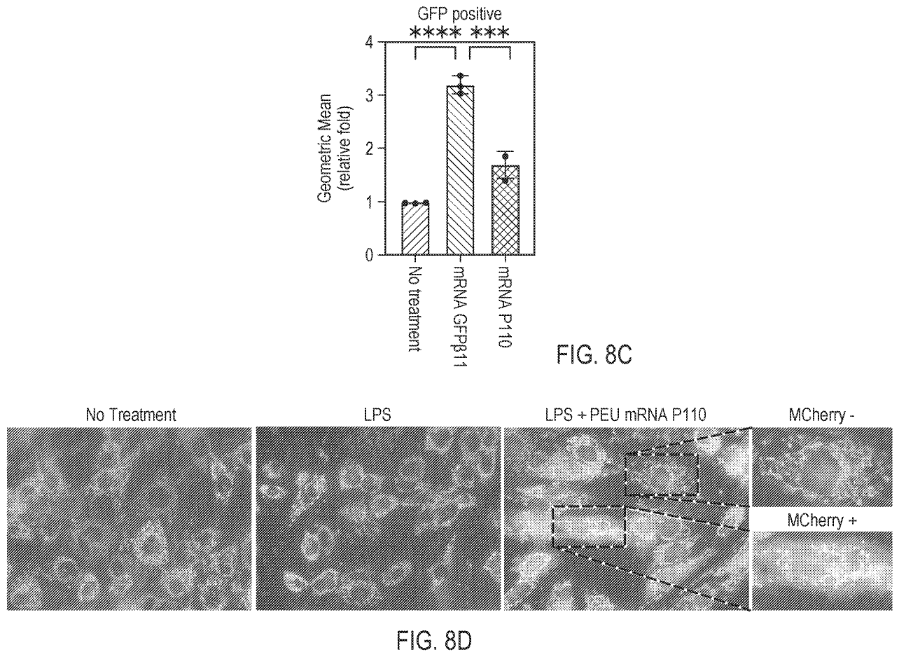

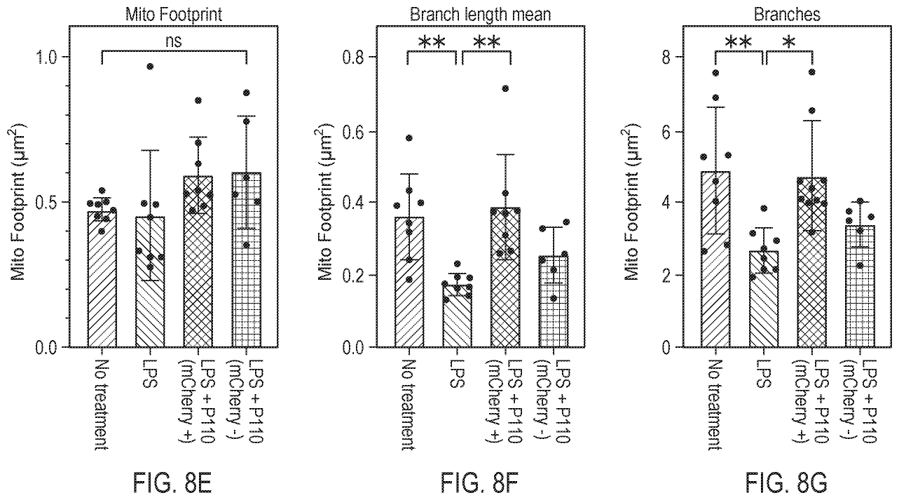

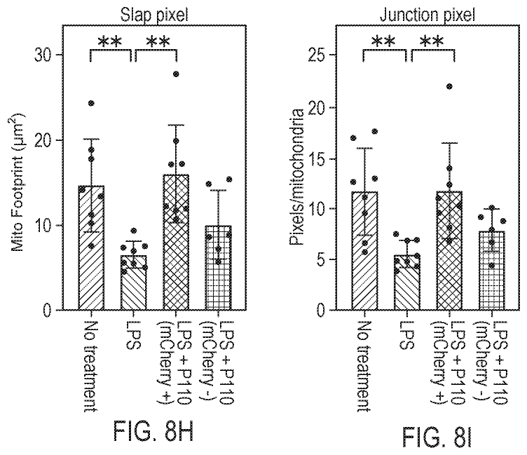

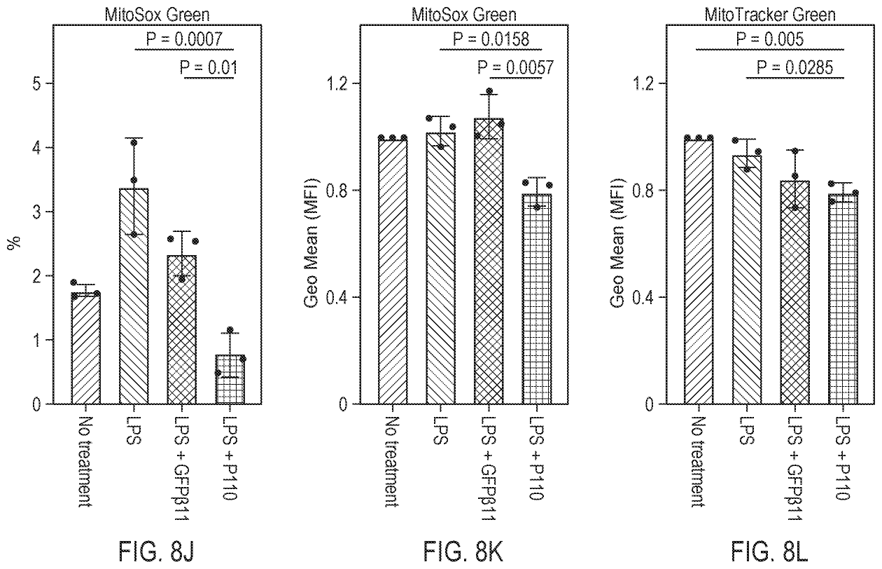

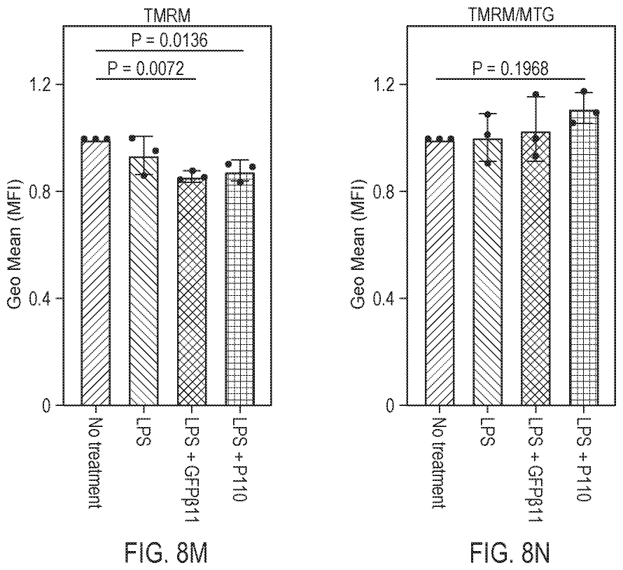

A peptide expression unit, mCherry-DHFR-P2A-P110, which comprises codon-optimized P110 (FIG. 2C) was transcribed using an in vitro transcription system and the resulting mRNA was introduced into H9c2 cardiomyocyte cells via lipofection. The efficiency of gene transfer of mRNA into naïve H9c2 cells was observed by fluorescence microscopy to be about 40% (FIG. 8A).

Next, GFPP1-10-expressing H9c2 cells were generated via transfection with a pLenti-GFPβ1-10-puro plasmid (FIG. 3). mRNA of a peptide expression unit comprising GFPP11 (mCherry-DHFR-P2A-GFPβ11) was introduced into GFPP1-10-expressing H9c2 cells via lipofection. About half of the resulting cells were mCherry-positive, and about 40% of the resulting mCherry-positive cells were also GFP-positive (FIG. 8B). In contrast, GFPP1-10-expressing H9c2 cells lipofected with the mRNA of the P110-comprising construct did not express GFP (FIGS. 8B and 8C).

Next, lipopolysaccharide (LPS) was used as an external stressor to trigger hyperfission. Briefly, H92c cells were seeded on day 0 and lipofected with an mRNA of a peptide expression unit comprising P110 coding sequence on day 1 and treated with TMP. After 12 hours, the medium was changed, and the cells were treated with LPS for 12 hours and mitochondrial morphology of cells was evaluated using mitotracker green staining and Mitochondrial Network Analysis (MiNA). LPS-induced changes in mitochondrial reactive oxygen species (ROS) production were evaluated using MitoSOX staining. LPS-induced changes in mitochondrial membrane potential were evaluated using TMRM staining. For the purposes of this assessment, mCherry-positive cells were assumed to express transgenic P110, whereas mCherry-negative cells were assumed to lack transgenic P110 and were assessed separately (FIG. 8D). The results showed that mCherry-positive cells reversed indications of hyperfission in terms of branching length, area of branching points, and number of branching points (FIGS. 8D-81), suggesting P110 expression in these cells suppressed hyperfission triggered by LPS. LPS treatment was associated with an increase in mitochondrial ROS, which was inhibited in P110-expressing cells (FIGS. 8J-8K), and with an increase in mitochondrial membrane potential per unit volume (FIGS. 8L-8N). Taken together, expression of P110 in cells suppressed mitophagy-related changes induced by LPS treatment.

### 8.5. Example 5: Gene Transfer of P110 Peptide Using Peptide Expression Unit mRNA Suppresses Doxorubicin-Induced Apoptosis

The anti-apoptotic effect of peptide expression unit mRNA comprising the P110 coding sequence was assessed in H9c2 cells upon doxorubicin exposure, a treatment that induces mitochondrial stress, hyperfission, and cell death. Briefly, H92c cells were seeded on day 0 and lipofected with an mRNA of a peptide expression unit comprising P110 coding sequence on day 1 and treated with TMP. After 12 hours, the medium was changed, and the cells were treated with doxorubicin for 3 to 6 hours. As with LPS exposure assessments in Example 4, the effect of P110 on hyperfission was assessed via mitotracker green staining, followed by MiNA.

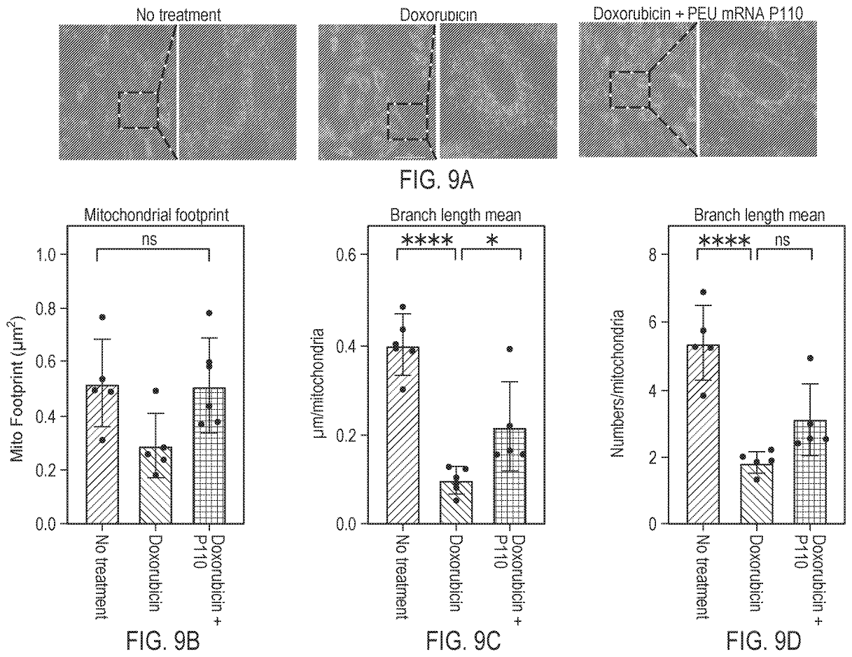

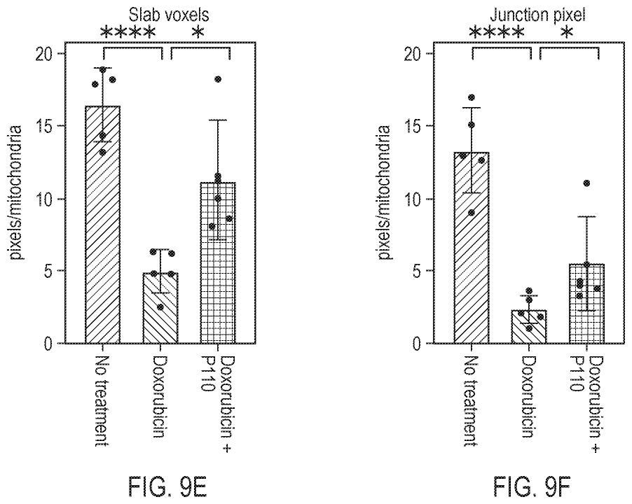

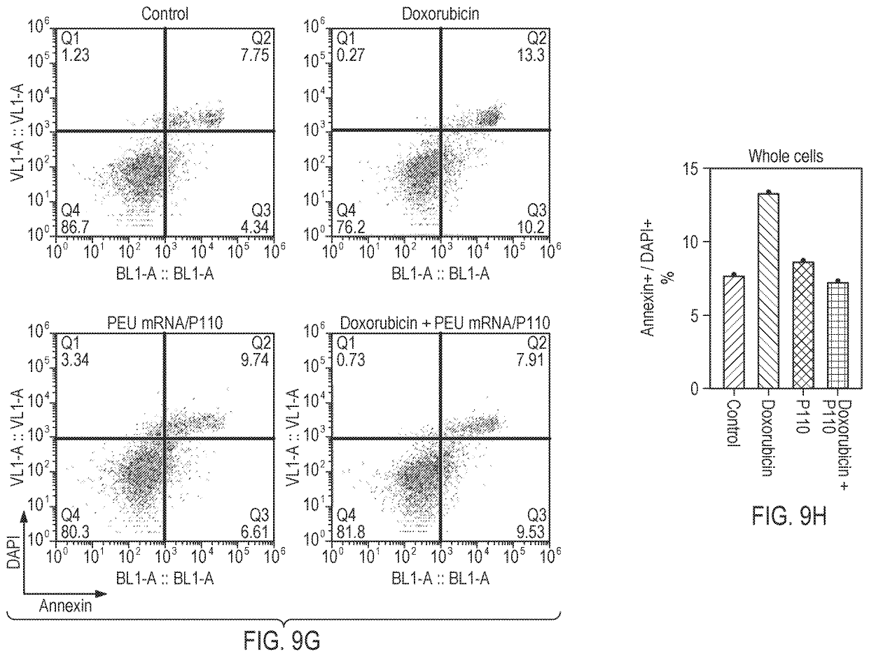

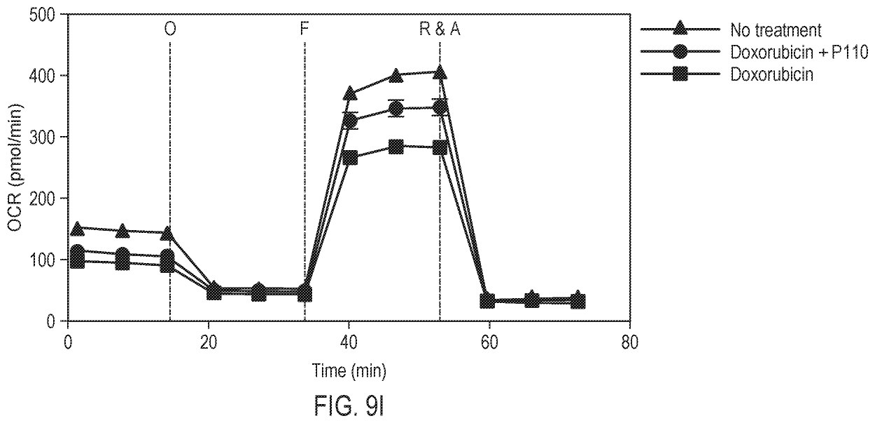

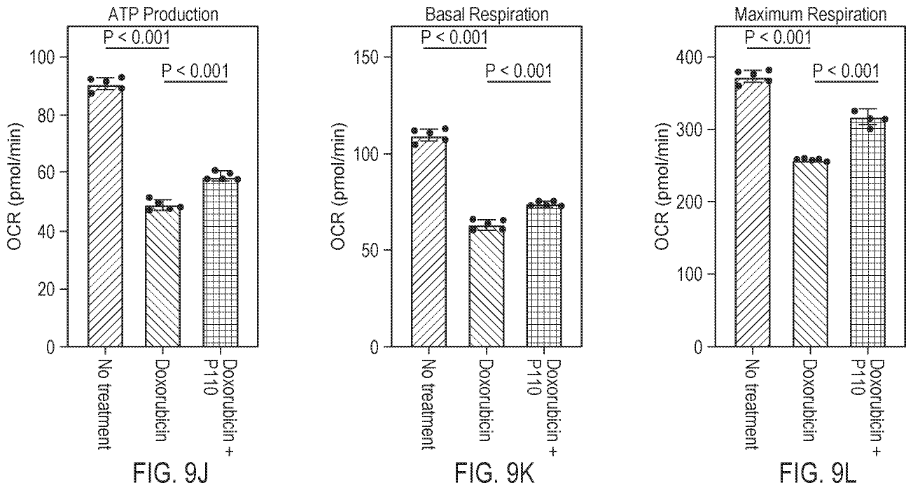

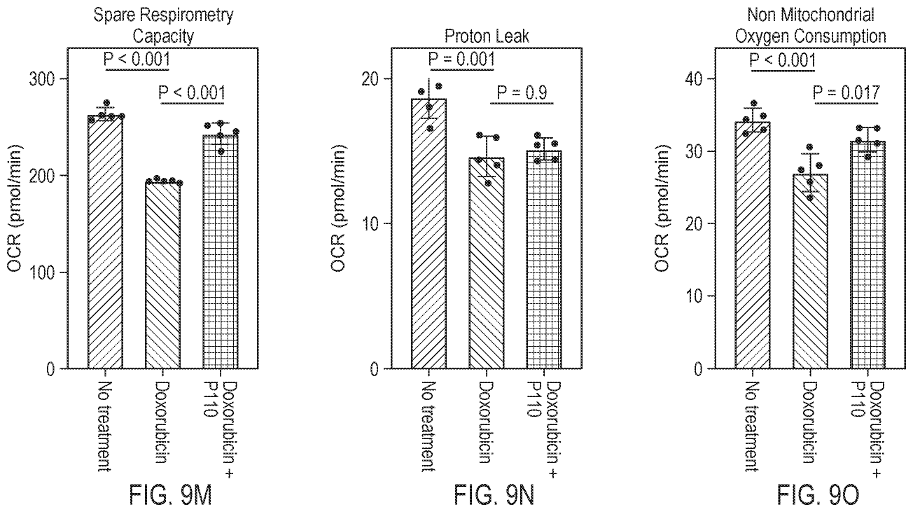

The increase in doxorubicin-induced fission was accompanied by an increase in footprint, a measure of mitochondrial mass, due to the expression of P110 from mRNA expression units, and by an increase in the length, number, and area of branching (FIGS. 9A-9F). Cell death was assessed by quantifying DAPI/annexin5 double positive cells as cell death markers using FACS (FIG. 9G), wherein the addition of doxorubicin resulted in cell death in almost 15% of cells, while cell death in cells transfected with the P110 mRNA expression unit was reduced to about 7% of all cells (FIG. 9H). These results indicate that peptide expression of P110 in cells lipofected with a peptide expression unit mRNA comprising the P110 coding sequence has effective anti-apoptotic activity.

Next, the effect of P110 expression on mitochondrial hyperfission was evaluated in the context of mitochondrial respiratory function. Briefly, oligomycin (2 μM), carbonyl cyanide p-trifluoromethoxyphenyl hydrazone (FCCP, 2 μM) and rotenone/antimycin A (0.5 μM), which were adjusted using the reagents in the Seahorse XF Cell Mito Stress Test Kit (103015-100, Agilent Technologies), were sequentially added to each well with 1×10⁵ cells after baseline measurements with an XFe96 extracellular flux analyzer (Agilent Technologies, Santa Clara, CA, USA), and data were presented as oxygen consumption rate (pmol/min). Doxorubicin treatment decreased mitochondrial respiration and this decrease was attenuated in P110-expressing cells (FIG. 9I). Doxorubicin treatment significantly decreased basal respiration, maximum respiration, spare respiratory capacity, and ATP production in control cells (FIGS. 9J-9M) and also significantly decreased non-mitochondrial oxygen consumption (FIG. 9O). P110-expressing cells displayed a significant attenuation of the doxorubicin-induced decreases in basal respiration, maximum respiration, and ATP production (FIGS. 9J-9L). Similarly, P110-expressing cells displayed a significant attenuation of doxorubicin-induced decrease in non-mitochondrial oxygen consumption (FIG. 9O). P110 expression restored spare respiratory capacity to a level comparable to that observed in untreated control cells (FIG. 9M). Doxorubicin-induced decrease in proton leakage was slightly improved by P110 expression (FIG. 9N).

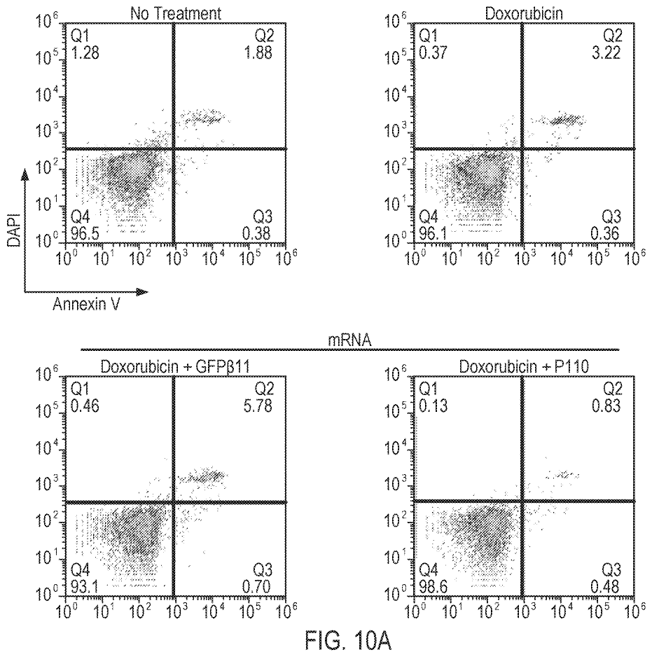

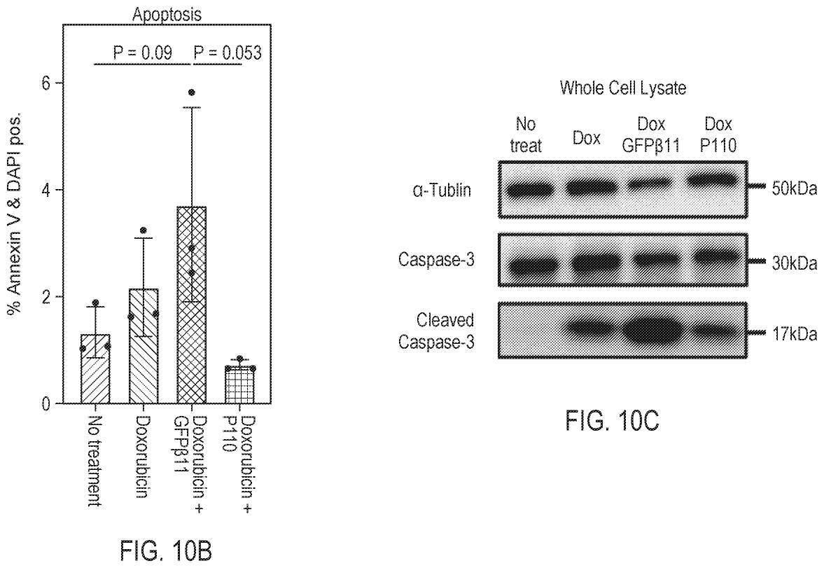

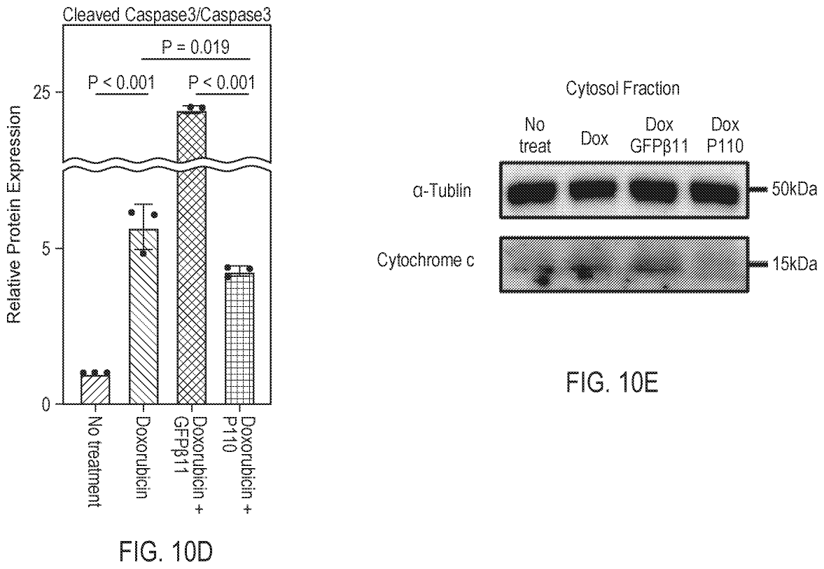

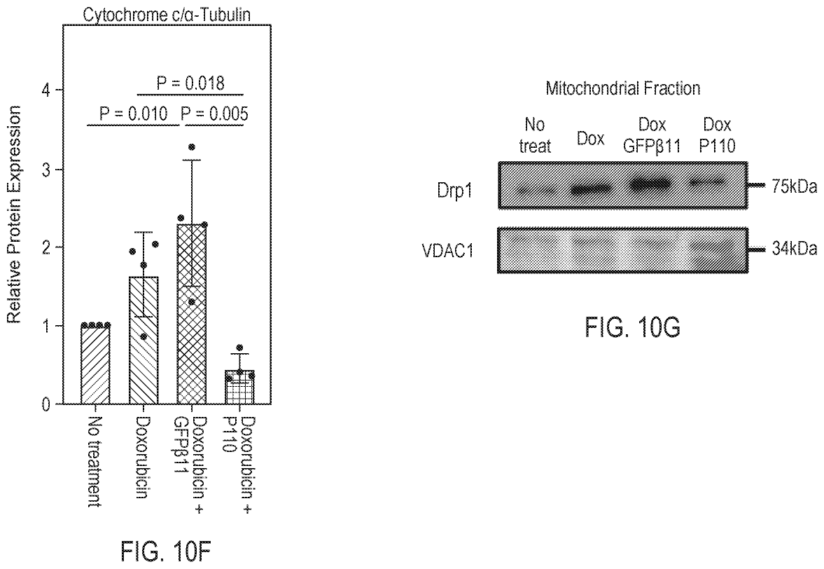

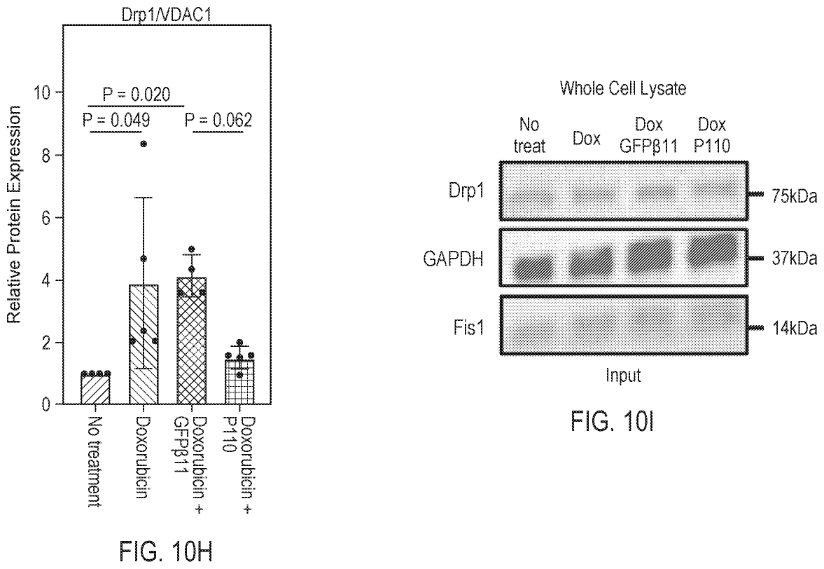

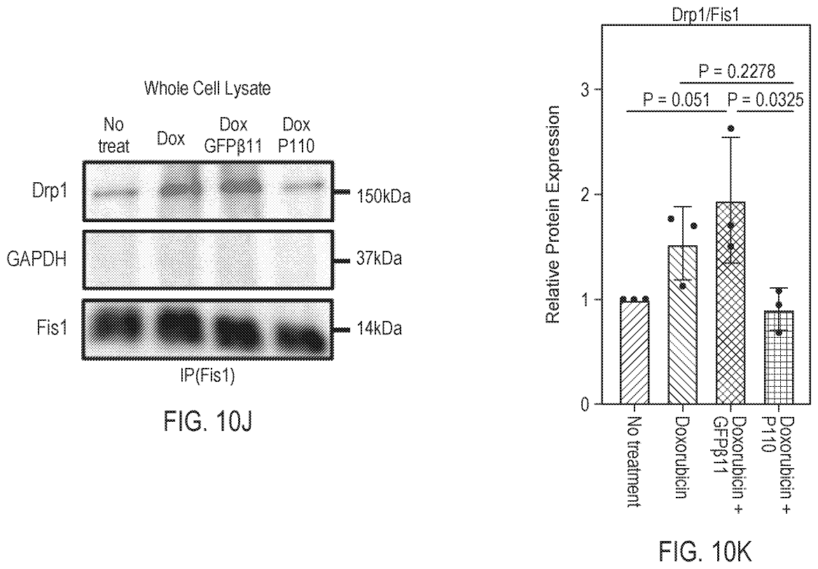

Doxorubicin treatment-induced apoptosis was assessed via FACS analysis of cells stained with the nuclear marker DAPI and the apoptotic marker Annexin V. Doxorubicin-treated control cells and GFPβ11-expressing cells displayed similar levels of apoptosis induction and there was evidence of harm at the level of gene transfer (FIGS. 10A-10B). P110-expressing cells counteracted doxorubicin-induced apoptosis as well as gene transfer-associated induction of cell death (FIGS. 10A-10B). Next, doxorubicin-induced activation of caspase-3, which is a cysteine-aspartic acid protease that plays a central role in the execution-phase of cell apoptosis, was evaluated. Although mRNA cell transfection strongly induced expression of cleaved caspase-3 over doxorubicin exposure, transfection of cells with P110 reduced the effects associated both with the gene transfer method and doxorubicin (FIGS. 10C-10D).

Hyperfission-associated release of cytochrome c from mitochondria triggers apoptosis; therefore, the release of cytochrome c in response to doxorubicin was evaluated. The release of cytochrome c by mRNA gene transfer was not as potent as cleaved caspase-3 induction but further enhanced doxorubicin-induced cytochrome c release, while transfection of cells with P110 completely suppressed cytochrome c release (FIGS. 10E-10F). This observation indicated that the mitochondrial hyperfission-associated apoptosis was completely suppressed by P110 expression, whereas non-mitochondrial apoptotic pathways were unaffected by P110. Given that P110 inhibits the recruitment of Drp1 to the mitochondrial membrane during fission, Drp1 in mitochondrial fractions were evaluated to determine whether the introduction of P110 mRNA affected this mechanism. The results showed that while doxorubicin exposure caused strong accumulation of Drp1 in mitochondria, P110 treatment significantly prevented Drp1 aggregation, maintaining it at almost the same level as in the control group (FIGS. 10G-10H).

Association of Drp1 and Fis1 is an intracellular PPI that is associated with mitochondrial dysfunction. To determine whether interaction of Fis1 and Drp1 was associated with the accumulation of Drp1 at the mitochondrial membrane, immunoprecipitation with Fis1 followed by immunoblotting with Drp1 was performed. Drp1 expression was significantly enhanced by doxorubicin treatment in non-transfected and GFPP11-transfected cells. On the other hand, Drp1 was restored to almost control levels with little aggregation in P110-transfected cells (FIGS. 10I-10K). In conclusion, P110 mRNA gene transfer inhibited the association of Drp1-Fis1, an intracellular PPI, preventing hyperfission that contributes to pathogenesis, which in turn, reduced cell death.

### 9. Citation of References

All publications, patents, patent applications and other documents cited in this application are hereby incorporated by reference in their entireties for all purposes to the same extent as if each individual publication, patent, patent application or other document were individually indicated to be incorporated by reference for all purposes. In the event that there is an inconsistency between the teachings of one or more of the references incorporated herein and the present disclosure, the teachings of the present specification are intended.

What is claimed is:

1. A mRNA molecule encoding:
(a) a destabilizing domain (DD);
(b) a translational separator; and
(c) a peptide of interest.

2. The mRNA of claim 1, wherein the DD is a protein, a peptide, or a peptide fragment that is rapidly degraded upon translation of the mRNA in the absence of a stabilizing molecule.

3. The mRNA of claim 1 or claim 2, wherein the DD-encoding sequence enhances ribosomal targeting of the mRNA as compared to a mRNA encoding the translational separator and the peptide of interest but not the destabilizing domain.

4. The mRNA of any one of claims 1 to 3, wherein the DD is a dihydrofolate reductase (DHFR), a FK506-binding protein (FKBP), a carbonic anhydrase 2 (CA2)-derived DD, a phosphodiesterase 5 (PDE5)-derived DD, a peroxisome proliferator-activated receptor gamma (PPAR gamma)-derived DD, a NRH:quinone oxidoreductase 2 (NQO2)-derived DD, a human estrogen receptor ligand binding domain (ERLBD)-derived DD, or an UnaG-derived DD.

5. The mRNA of any one of claims 1 to 4, wherein the nucleotide sequence encoding DD is codon-optimized for expression in human cells.

6. The mRNA of any one of claims 1 to 5, the DD is a DHFR (e.g., an E. coli DHFR (ecDHFR)), optionally wherein the nucleotide sequence encoding the DD encodes a DD comprising the amino acid sequence of SEQ ID NO:1 or SEQ ID NO:2.

7. The mRNA of any one of claims 1 to 6, wherein the nucleotide sequence encoding the translational separator is located 3′ to the nucleotide sequence encoding the DD and 5′ to the nucleotide sequence encoding the peptide of interest.

8. The mRNA of any one of claims 1 to 7, wherein the translational separator is a self-cleaving peptide.

9. The mRNA of claim 8, wherein the self-cleaving peptide is a P2A, E2A, F2A, or T2A self-cleaving peptide.

10. The mRNA of claim 9, wherein the self-cleaving peptide is a P2A self-cleaving peptide, optionally wherein the amino acid sequence of the P2A self-cleaving peptide comprises the amino acid sequence of SEQ ID NO:15, SEQ ID NO:16, or SEQ ID NO:17.

11. The mRNA of any one of claims 1 to 10, wherein the nucleotide sequence encoding the self-cleaving peptide is codon-optimized for expression in human cells.

12. The mRNA of any one of claims 1 to 11, wherein the nucleotide sequence encoding the peptide of interest is codon-optimized for expression in human cells.

13. The mRNA of any one of claims 1 to 12, wherein the peptide of interest is a peptide that cannot be effectively delivered into target cells via conventional drug delivery methods.

14. The mRNA of any one of claims 1 to 13, wherein the peptide of interest is 2 to 150 amino acids in length.

15. The mRNA of any one of claims 1 to 14, wherein the peptide of interest is a therapeutic peptide.

16. The mRNA of any one of claims 1 to 15, wherein the peptide of interest is capable of inhibiting protein-protein interactions.

17. The mRNA of any one of claims 1 to 16, wherein the peptide of interest is an inhibitor of dynamin-1-related protein (Drp1), T-lymphokine-activated killer cell-originated protein kinase (TOPK), Sal-like protein 4 (SALL4), Ras, p53, protein phosphatase 2A (PP2A), signal transducer and activator of transcription 3 (STAT3), Yes-associated protein (YAP), a Bcl-2 family protein, NOTCH, an estrogen receptor, microtubule-associated protein light chain 3 (LC3), or MDM2/MDMX.

18. The mRNA of any one of claims 1 to 17, wherein the peptide of interest is a peptide listed in Table 2.

19. The mRNA of any one of claims 1 to 18, wherein the peptide of interest is a Drp1 inhibitor.

20. The mRNA of any one of claims 1 to 19, wherein the peptide of interest is P110.

21. The mRNA of claim 20, wherein the nucleotide sequence encoding P110 is codon-optimized for expression in human cells.

22. The mRNA of claim 20 or claim 21, wherein the nucleotide sequence encoding P110 comprises the nucleotide sequence of SEQ ID NO:25.

23. The mRNA of any one of claims 1 to 22, wherein the mRNA molecule encodes a single copy of the peptide of interest.

24. The mRNA of any one of claims 1 to 22, wherein the mRNA molecule encodes two or more copies of the peptide of interest.

25. The mRNA of any one of claims 1 to 24, wherein the mRNA further encodes a second peptide of interest that is different from the first peptide of interest.

26. The mRNA of claim 25, wherein each sequence encoding a peptide of interest is separated by a translational separator, optionally wherein each translational separator is independently selected from translational separators described in claims 9 to 10.

27. The mRNA of any one of claims 1 to 26, which further encodes a marker protein.

28. A DNA encoding the mRNA of any one of claims 1 to 27.

29. The DNA of claim 28, which further comprises one or more regulatory elements, optionally wherein one or more regulatory elements comprise a TATA box, a CAAT box, a GC box, a promoter or a combination thereof.

30. The DNA of claim 28, which further comprises a promoter operably linked to the sequence encoding the mRNA.

31. The DNA of any one of claims 28 to 30, which further comprises a polyadenylation signal sequence 3′ to the sequence encoding the peptide of interest.

32. The DNA of any one of claims 28 to 31, which is an expression plasmid or a viral genome, optionally wherein the viral genome is an AAV genome.

33. A particle comprising the mRNA of any one of claims 1 to 27 or the DNA of any one of claims 28 to 32, which is optionally a viral particle or a lipid particle.

34. A pharmaceutical composition comprising the mRNA of any one of claims 1 to 27, the DNA of any one of claims 28 to 32, or the particle of claim 33, and one or more excipients.

35. A host cell comprising the mRNA of any one of claims 1 to 27, the DNA of any one of claims 28 to 32, or the particle of claim 33.

36. A method of introducing a mRNA into a cell, comprising contacting the cell with the mRNA of any one of claims 1 to 27, the DNA of any one of claims 28 to 32, the particle of claim 33, or the pharmaceutical composition of claim 34.

37. A method of expressing a peptide of interest in a cell, comprising contacting the cell with the mRNA of any one of claims 1 to 27, the DNA of any one of claims 28 to 32, the particle of claim 33, or the pharmaceutical composition of claim 34.

38. A method of inhibiting Drp1/Fis1 binding in a cell, comprising contacting the cell with an mRNA of any one of claims 1 to 27 that encodes a Drp1 inhibitor such as P110, the DNA of any one of claims 28 to 32 that encodes a Drp1 inhibitor such as P110, or the pharmaceutical composition of claim 34 that comprises mRNA or DNA encoding a Drp1 inhibitor such as P110.

39. A method of inhibiting mitochondrial hyperfission in a cell, comprising contacting the cell with an mRNA of any one of claims 1 to 27 that encodes a Drp1 inhibitor such as P110, the DNA of any one of claims 28 to 32 that encodes a Drp1 inhibitor such as P110, or the pharmaceutical composition of claim 34 that comprises mRNA or DNA encoding a Drp1 inhibitor such as P110.

40. A method of inhibiting apoptosis of the mitochondrial pathway in a cell, comprising contacting the cell with an mRNA of any one of claims 1 to 27 that encodes a Drp1 inhibitor such as P110, the DNA of any one of claims 28 to 32 that encodes a Drp1 inhibitor such as P110, or the pharmaceutical composition of claim 34 that comprises mRNA or DNA encoding a Drp1 inhibitor such as P110.

41. A method of inducing apoptosis in a cancer cell, comprising contacting the cell with an mRNA of any one of claims 1 to 27 that encodes a peptide that inhibits an Bcl-2 family protein, TOPK, SALL4, Ras, p53, PP2A, STAT3, or YAP, the DNA of any one of claims 28 to 32 that encodes a peptide that inhibits an Bcl-2 family protein, TOPK, SALL4, Ras, p53, PP2A, STAT3, or YAP, or the pharmaceutical composition of claim 34 that comprises mRNA or DNA encoding a peptide that inhibits an Bcl-2 family protein, TOPK, SALL4, Ras, p53, PP2A, STAT3, or YAP.

42. A method of treating a neurodegenerative disease, cardiovascular disease, or diabetes, comprising administering to the subject in need thereof a therapeutically effective amount of the mRNA of any one of claims 1 to 27 that encodes P110, the DNA of any one of claims 28 to 32 that encodes P110, or the pharmaceutical composition of claim 34 that comprises mRNA or DNA encoding P110.

43. A method of treating a neurodegenerative disease, cardiovascular disease, or diabetes, comprising administering to the subject in need thereof a therapeutically effective amount of the mRNA of any one of claims 1 to 27 that encodes a Drp1 inhibitor such as P110, the DNA of any one of claims 28 to 32 that encodes a Drp1 inhibitor such as P110, or the pharmaceutical composition of claim 34 that comprises mRNA or DNA encoding a Drp1 inhibitor such as P110.
